(19) United States
(12) Patent Application Publication
(10) Pub. No.: US 2026/0183242 A1
(43) Pub. Date: Jul. 2, 2026

(54) FORMULATIONS COMPRISING A COMBINATION OF ACID-NEUTRALIZING POLYMERIC AND NON-POLYMERIC SUBSTANCES FOR ORAL ADMINISTRATION OF ACTIVE AGENTS

(71) Applicant: Entera Bio Ltd., Jerusalem (IL)

(72) Inventors: Gregory BURSHTEIN, Modiin (IL); Constantin ITIN, Mevaseret-Zion (IL); Hillel GALITZER, Yad Binyamin (IL)

(21) Appl. No.: 19/544,782

(22) Filed: Feb. 19, 2026

Related U.S. Application Data
(63) Continuation of application No. PCT/IL2024/050877, filed on Aug. 30, 2024.
(60) Provisional application No. 63/535,495, filed on Aug. 30, 2023.

(51) Int. Cl. A61K 9/20; A61K 38/26; A61K 38/29

(52) U.S. Cl. CPC A61K 9/2013; A61K 9/2009; A61K 9/2054; A61K 9/2059; A61K 38/26; A61K 38/29

(57) ABSTRACT

A pharmaceutical composition is described herein, which comprises a therapeutically active agent, an absorption enhancer, a polymer comprising a plurality of alkaline groups, and a non-polymeric (small molecule) proton-accepting agent. The absorption enhancer is preferably a substituted fatty acid or a salt thereof. Further described herein are methods of treating a condition treatable by the therapeutically active agent, comprising orally administering the pharmaceutical composition.

### RELATED APPLICATION/S

This application claims the benefit of priority under 35 USC § 119 (e) of U.S. Provisional Patent Application No. 63/535,495 filed on 30 Aug. 2023, the contents of which are incorporated herein by reference in their entirety.

### FIELD AND BACKGROUND OF THE INVENTION

The present invention, in some embodiments thereof, relates to drug delivery, and more particularly, but not exclusively, to formulations and/or systems for oral administration of therapeutically active agents such as, but not limited to, therapeutically active peptides and/or proteins.

Oral administration of peptide and/or protein therapeutically active agents (pharmaceuticals) is problematic due to degradation of peptides and/or proteins in the digestive system and poor absorption of large molecules.

Qi & Ping [J Microencapsulation 2004, 21:37-45] describe administration of enteric microspheres containing insulin with SNAC (sodium 8-N-(2-hydroxybenzoyl)aminocaprylate). The enteric microspheres are for protecting the insulin from digestive enzymes of the stomach and small intestine, and the SNAC is for enhancing absorption.

U.S. Patent Application Publication No. 2011/0142800 describes compositions for oral administration of a protein, comprising a protein having a molecular weight of up to 100,000 Da, a protease inhibitor, and an absorption enhancer, such as SNAC, N-(10-[2-hydroxybenzoyl]amino) decanoic acid (SNAD), 8-[N-(2-hydroxy-4-methoxybenzoyl)amino]caprylic acid (4-MOAC), 8-[N-(2-hydroxy-5-chlorobenzoyl)amino]caprylic acid (5-CNAC) and 4-[(4-chloro-2-hydroxy-benzoyl)amino]butanoic acid (4-CNAB) and sodium salts thereof.

International Patent Application Publication No. WO 00/48589 describes solid oral dosage forms comprising a heparin drug in admixture with SNAC or SNAD for facilitating absorption and/or enhancing bioavailability of the heparin drug, wherein the heparin drug is reported to protect the SNAC or SNAD from precipitation during transit through acidic regions of the gastrointestinal (GI) tract.

International Patent Application Publication No. WO 2016/128974 describes a pharmaceutical composition for oral administration comprising a therapeutically active agent, SNAC and at least one antacid compound; as well as a pharmaceutical composition unit dosage form for oral administration which comprises a core comprising a therapeutically active agent and SNAC, and an external layer comprising at least one of an antacid compound and a protease inhibitor.

Buckley et al. [Sci Transl Med 2018, 10: eaar7047] reports that upon oral administration of a tablet comprising the peptide agent semaglutide and SNAC, absorption of the semaglutide takes place in the stomach and is confined to an area in close proximity to the tablet surface, and that the SNAC protects against enzymatic degradation via local buffering actions and only transiently enhances absorption.

International PCT Patent Application Publication No. WO 2021/089761 describes pharmaceutical compositions comprising a PCSK9 inhibitor, SNAC and a hydrotrope capable of enhancing the solubility of SNAC at least 2-fold, such as Nipecotamide, Nicotinamide, p-hydroxybenzoic acid sodium, N,N dimethyl urea, N,N dimethyl benzamide, N,N diethyl nicotinamide, Sodium salicylate, Resorcinol, Sodium benzoate, Sodium Xylenesulfonate, Sodium p-toluenesulfonate, 1-Methylnicotinamide, Pyrogallol, Pyrocathecol, Epigallocatechin gallate, Tannic acid and Gentisic acid sodium salt hydrate.

U.S. Pat. No. 10,583,177 describes a pharmaceutical composition for oral administration comprising a therapeutic agent, SNAC, and at least one antacid capable of enhancing the solubility of SNAC, such as calcium citrate, calcium gluconate, sodium gluconate, sodium citrate, sodium hydroxide, potassium gluconate, potassium citrate, potassium hydroxide, magnesium gluconate, magnesium citrate, magnesium hydroxide, magnesium oxide, aluminum gluconate, aluminum citrate, and aluminum hydroxide.

International PCT Patent Application Publication No. WO 2023/161934 describes a pharmaceutical composition comprising a therapeutically active agent, an absorption enhancer, and a polymer comprising a plurality of alkaline groups which are effective at protecting the absorption enhancer from stomach acid, as well as enhancing bioavailability of a therapeutically active agent in compositions comprising the absorption enhancer and the therapeutically active agent, such as sodium starch glycolate and/or croscarmellose sodium.

Sodium carboxymethylcellulose (CMC) is prepared by modifying cellulose with chloroacetic acid, and may be crosslinked or non-crosslinked. In its crosslinked form, wherein crosslinking is effected by formation of ester bonds between carboxymethyl groups and cellulose, it is commonly referred to as croscarmellose sodium (CCS) or sodium croscarmellose, and is used as a superdisintegrant in pharmaceutical formulations.

Similarly, sodium starch glycolate (SSG) can be prepared by modifying starch with chloroacetic acid to form carboxymethyl groups. The sodium starch glycolate may be further crosslinked by phosphate groups (—O—P(═O)(—O⁻)—O—) between starch backbones, and used as a superdisintegrant.

Additional background art includes Qi et al. [Acta Pharm Sinica 2004, 39:844-848]; International Patent applications Publication Nos. WO 00/50386, WO 01/32130, WO 01/32596, WO 03/045306, WO 03/045331, WO 2006/076692, WO 2006/084164, WO 2007/121471, WO 2010/020978, WO 2012/080471, WO 2016/128970, WO 2016/128971, WO 2016/128972, WO 2016/128973, WO 2016/128974 and WO 2018/033927; Japanese Patent Application Nos. 2005281231 and 2006111558; U.S. Pat. No. 8,110,547; and U.S. Patent Application Publication Nos. 2006/0234913, 2007/0087957, and 2013/0224300.

### SUMMARY OF THE INVENTION

According to an aspect of some embodiments of the invention, there is provided a pharmaceutical composition comprising a therapeutically active agent, an absorption enhancer, a polymer comprising a plurality of alkaline groups and a non-polymeric (small molecule) proton-accepting agent (a non-polymeric acid-neutralizing substance).

According to an aspect of some embodiments of the invention, there is provided a pharmaceutical composition comprising a therapeutically active agent, an absorption enhancer, a polymer comprising a plurality of alkaline groups, and at least one proton-accepting small molecule (non-polymeric) agent (non-polymeric acid-neutralizing agent or substance), wherein a concentration of the polymer in the composition is at least 5, or at least 10 weight percent of the total weight of the composition and a concentration of the proton accepting agent is such that a mol ratio of the at least one proton accepting agent to the absorption enhancer is at least 0.1:1 or ranges from 0.1:1 to 5:1, or from 0.1:1 to 2:1, or from 0.2:1 to 2:1, or from 0.5:1 to 1:1, the absorption enhancer being a substituted fatty acid or a salt thereof.

According to some of any of the embodiments described herein, the alkaline groups in the polymer are carboxylate groups and/or amine groups.

According to some of any of the embodiments described herein, at least a portion of the alkaline groups in the polymer are carboxylate groups.

According to some of any of the embodiments described herein, at least a portion of the carboxylate groups in the polymer are in a form of a pharmaceutically acceptable salt.

According to some of any of the embodiments described herein, at least a portion of the carboxylate groups in the polymer are in a form of a sodium salt.

According to some of any of the embodiments described herein, the absorption enhancer is selected from NAC (8-N-(2-hydroxybenzoyl)aminocaprylic acid), NAD (10-N-(2-hydroxybenzoyl)aminodecanoic acid), 5-CNAC (8-N-(5-chlorosalicyloyl)aminocaprylic acid), 4-MOAC (8-N-(2-hydroxy-4-methoxybenzoyl)aminocaprylic acid), 4-CNAB (4-N-(2-hydroxy-4-chlorobenzoyl)aminobutanoic acid) and salts thereof.

According to some of any of the embodiments described herein, the absorption enhancer comprises NAC or a salt thereof.

According to some of any of the embodiments described herein, a concentration of the absorption enhancer is at least 20, at least 30, at least 40 or at least 50 weight percent, of the total weight of the composition.

According to some of any of the embodiments described herein, a concentration of the polymer is at least 10 or at least 20, weight percent of the total weight of the composition.

According to some of any of the embodiments described herein, a total concentration of the absorption enhancer and the polymer is at least 30, or at least 40, or at least 50, weight percent, of the total weight of the composition

According to some of any of the embodiments described herein, a concentration of the alkaline groups in the polymer in the composition is at least 0.1 millimoles per gram.

According to some of any of the embodiments described herein, the polymer is a crosslinked polymer.

According to some of any of the embodiments described herein, the polymer comprises a polysaccharide.

According to some of any of the embodiments described herein, the polysaccharide is selected from a starch derivative and a cellulose derivative.

According to some of any of the embodiments described herein, the polymer comprises carboxymethyl groups.

According to some of any of the embodiments described herein, the polymer is characterized by a pKa in a range of from 1.2 to 7.5.

According to some of any of the embodiments described herein, the polymer is sodium starch glycolate (SSG) and/or croscarmellose sodium.

According to some of any of the embodiments described herein, the at least one small proton-accepting agent independently features a diffusion kinetic in the stomach that is similar to that of the absorption enhancer.

According to some of any of the embodiments described herein, the at least one small proton-accepting agent independently has a molecular weight in a range of from 20% to 200%, or from 20% to 120%, or from 20% to 100%, of the molecular weight of the absorption enhancer.

According to some of any of the embodiments described herein, the at least one small proton-accepting agent independently has a molecular weight lower than 1,000, or lower than 500 grams/mol.

According to some of any of the embodiments described herein, the at least one small proton-accepting agent independently features pKa of at least 5, e.g., of from 5 to 12, or from 6 to 12.

According to some of any of the embodiments described herein, the at least one small proton-accepting agent independently features pKa higher by at least 0.5, or by at least 1, than a pKa of the absorption enhancer.

According to some of any of the embodiments described herein, the at least one small proton accepting agent is selected from an antacid, an amino acid and a fatty acid salt.

According to some of any of the embodiments described herein, the antacid is selected from the group consisting of calcium carbonate, calcium gluconate, calcium citrate, sodium carbonate, sodium bicarbonate, sodium hydroxide, potassium carbonate, potassium bicarbonate, potassium gluconate, potassium citrate, potassium hydroxide, magnesium carbonate, magnesium gluconate, magnesium citrate, magnesium hydroxide, magnesium oxide, aluminum carbonate, aluminum gluconate, aluminum citrate, aluminum hydroxide, and any combinations thereof.

According to some of any of the embodiments described herein, the at least one small proton-accepting agent comprises sodium carbonate.

According to some of any of the embodiments described herein, the at least one small proton-accepting agent comprises potassium carbonate.

According to some of any of the embodiments described herein, the at least one small proton-accepting agent comprises an amino acid featuring a pKa of at least 5, preferably of at least 6.

According to some of any of the embodiments described herein, the amino acid is selected from histidine, arginine, lysine, an analog thereof and any combinations thereof.

According to some of any of the embodiments described herein, the amino acid is arginine.

According to some of any of the embodiments described herein, the at least one small proton-accepting agent comprises a salt of a fatty acid.

According to some of any of the embodiments described herein, the fatty acid is a medium-chain fatty acid, being of from 4 to 12, or from 6 to 12, or from 6 to 10, or from 8 to 10 carbon atoms in length.

According to some of any of the embodiments described herein, the salt of a fatty acid is selected from sodium octanoate and sodium decanoate.

According to some of any of the embodiments described herein, the at least one small proton-accepting agent comprises a salt of a fatty acid and an antacid.

According to some of any of the embodiments described herein, the at least one small proton-accepting agent comprises a salt of a fatty acid and an amino acid.

According to some of any of the embodiments described herein, a total concentration of the at least one small proton-accepting agent in the composition is at least 5, or at least 10, or at least 20, weight percent of the total weight of the composition.

According to some of any of the embodiments described herein, a Cmax and/or a bioavailability of the composition upon oral administration is at least 50% greater than a Cmax and/or a bioavailability of a corresponding composition without the polymer and the proton accepting agent.

According to some of any of the embodiments described herein, a Cmax and/or a bioavailability of the composition upon oral administration is at least 20%, or at least 50% greater than a Cmax and/or a bioavailability of a corresponding composition without the proton accepting agent.

According to some of any of the embodiments described herein, the therapeutically active agent has a molecular weight in a range of 0.5 kDa to 100 kDa.

According to some of any of the embodiments described herein, the therapeutically active agent is a BCS Class III agent.

According to some of any of the embodiments described herein, the therapeutically active agent is a polypeptide or nucleic acid.

According to some of any of the embodiments described herein, the polypeptide is selected from an antibody, an antipathogenic peptide, an anti-viral peptide, a blood clotting factor, a C-type natriuretic peptide, a calcitonin, disitertide, an endomorphin, an erythropoietin, a gastric inhibitory polypeptide (GIP), a gastric inhibitory polypeptide receptor agonist, a glucagon, a glucagon receptor agonist, a GLP-1 (glucagon-like peptide-1), a GLP-1 receptor agonist, a GLP-2 (glucagon-like peptide-2), a GLP-2 receptor agonist, GLP-1 receptor/glucagon receptor dual agonist, a GIP receptor/GLP-1 receptor dual agonist, glucagon receptor/GIP receptor/GLP-1 receptor triple agonist, GLP-2 receptor/glucagon receptor dual agonist, GLP-2 receptor/GLP-1 receptor dual agonist, GLP-2 receptor/GIP receptor dual agonist, oxyntomodulin, a glucocerebrosidase, a gonadotropin, a gonadotropin releasing hormone, a gonadotropin releasing hormone receptor agonist, a gonadotropin releasing hormone receptor antagonist, a granulocyte-colony stimulating factor, a growth factor, a growth hormone, a somatostatin, a somatostatin receptor agonist, a growth hormone-releasing hormone, icatibant, insulin, an interferon, an interleukin, a kappa opioid receptor agonist, a leptin, an oxytocin, an oxytocin receptor agonist, a melanocortin, a melanocortin receptor agonist, a motilin, an omentin, a parathyroid hormone or a fragment thereof, a parathyroid hormone related protein, a peptide YY, a pituitary adenylate cyclase-activating peptide (PACAP), thymopentin, a thymosin, a TNF inhibitor, a vasoactive intestinal peptide, and a vasopressin, and analogs thereof, including long-acting analogs thereof, as described herein.

According to some of any of the embodiments described herein, the polypeptide is selected from desmopressin, salmon calcitonin, leuprolide, PTH, and octreotide, fragments thereof and analogs thereof, including long-acting analogs thereof.

According to some of any of the embodiments described herein, the polypeptide is selected from PTH a PTH fragment, and a long-acting analog of any of the foregoing.

According to some of any of the embodiments described herein, the polypeptide comprises teriparatide.

According to some of any of the embodiments described herein, the antibody comprises a single-domain antibody.

According to some of any of the embodiments described herein, the polypeptide is characterized by isoelectric point (pl) of at least 8.5.

According to some of any of the embodiments described herein, the therapeutically active agent is characterized by plasma half-life lower than 60 minutes or lower than 30 minutes, and an amount of the polymer featuring the alkaline groups ranges from 5 to 20, or from 5 to 15, or from 10 to 20, or from 10 to 15, weight percent of the total weight of the composition.

According to some of any of the embodiments described herein, the therapeutically active agent is characterized by plasma half-life higher than 30 minutes or higher than 60 minutes, and an amount of the polymer featuring the alkaline groups ranges from 15 to 30, or from 20 to 30, or from 15 to 25, or from 20 to 25, weight percent of the total weight of the composition.

According to some of any of the embodiments described herein, the composition is in a form of a unit dosage form.

According to some of any of the embodiments described herein, an amount of the alkaline groups in the unit dosage form is at least 0.03 millimoles.

According to some of any of the embodiments described herein, the unit dosage form comprises at least 20, or at least 30, or at least 50, mg of the absorption enhancer.

According to some of any of the embodiments described herein, the unit dosage form comprises at least 1, or at least 2, or at least 5, or at least 10, mg of the proton-accepting agent.

According to some of any of the embodiments described herein, the unit dosage form comprises one or more tablet(s).

According to an aspect of some embodiments of the invention, there is provided a composition as described herein in any of the respective embodiments, for use in the treatment of a condition treatable by the therapeutically active agent, the treatment comprising oral administration of the composition.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a graph showing the measured pH upon addition of SNAC to an HCl solution (pH 1.2), as a function of SNAC concentration.

FIG. 2 presents a graph showing the percentage of human parathyroid hormone (1-34) (PTH) (initial concentration 0.135 mg/ml) which remained upon incubation with pepsin (150 μg/ml) for 3 minutes at 37° C., as a function of pH.

FIGS. 3A-B present graphs showing the pH upon addition of sodium alginate, sodium carboxymethylcellulose (Na-CMC), sodium starch glycolate (SSG) or croscarmellose sodium (CCS) to an HCl solution (pH 1.2), as a function of concentration of the added polymer, measured using MP-103 pH-meter (MRC, Israel) equipped with ELC-10-00 electrode (MRC, Israel) (FIG. 3A) or with a thin electrode HI1083 (HANNA instruments Inc.) (FIG. 3B).

FIG. 4 presents a graph of median plasma concentration of hPTH(1-34) in rats as a function of time following oral administration of mini-tablets comprising 90 μg hPTH(1-34), SNAC and SBTI (soybean trypsin inhibitor), with 20% sodium starch glycolate (continuous line) or without sodium starch glycolate (dashed line).

FIG. 5 presents a graph of median plasma concentration of hPTH(1-34) in rats as a function of time following oral administration of mini-tablets comprising 90 μg hPTH(1-34) and SNAC, with 30% sodium starch glycolate (continuous line) or without sodium starch glycolate (dashed line).

FIGS. 6A-D present photographs of tablets made of the polymers sodium starch glycolate (SSG; FIG. 6A), sodium croscarmellose (CCS; FIG. 6B), sodium carboxymethyl cellulose (CMC-Na; FIG. 6C) and sodium alginate (Alg-Na; FIG. 6D), following swelling in 0.01 M HCl (pH=2), at room temperature, along with pH values measured at four points for each tablet (white triangles).

FIGS. 7A-B present photographs of tablets made of the polymers sodium starch glycolate (SSG; FIG. 7A), and sodium carboxymethyl cellulose (CMC-Na; FIG. 7B), following swelling in porcine gastric juice, at room temperature, along with pH values measured at four points for each tablet (white triangles).

FIG. 8 is a graph showing the median teduglutide plasma concentration following oral administration of tablets comprising 0.7 mg teduglutide (2.34 mg/Kg), SNAC (15 mg) and SSG (5.33 MG) to male Sprague-Dawley rats weighing about 299 grams, n=7.

FIG. 9 presents comparative plots showing the median plasma concentration (AUC) of an exemplary 1 kD therapeutic peptide following oral administration of tablets comprising the therapeutic peptide (0.1 mg/kg), SNAC and SSG 10% by weight (dashed line) or 30% by weight (black line), to male dogs (n=6 in each group). Statistical significance in the measured AUC values was found between the two groups (t-test, p<0.05).

FIGS. 10A-10B present comparative plots showing the SNAC soluble fraction percentage over a 30-minute period incubation at 37° C. in an acidic solution featuring pH of 1.6 (+/−0.1), from tablet formulations containing SNAC, SSG and Na₂CO₃ at 1:1, 0.5:1 and 0.25:1 mol ratio to SNAC, (FIG. 10A), and from tablet formulations containing SNAC, SSG and K₂CO₃ at 1:1, and 0.5:1 mol ratio to SNAC, (FIG. 10B), all compared to tablet formulations containing SNAC and SGG.

FIG. 11 presents comparative plots showing the SNAC soluble fraction percentage over a 30-minute period incubation at 37° C. in an acidic solution featuring pH of 1.6 (+/−0.1), from tablet formulations containing SNAC, SSG and sodium decanoate (C10) at 1:1, 0.5:1 and 0.25:1 mol ratio to SNAC, from tablet formulations containing SNAC, SSG and sodium octanoate (C8) at 1:1 mol ratio to SNAC, and from tablet formulations containing SNAC and SSG.

FIG. 12 presents comparative plots showing the SNAC soluble fraction percentage over a 30-minute period incubation at 37° C. in an acidic solution featuring pH of 1.6 (+/−0.1), from tablet formulations containing SNAC, SSG and Arginine (Arg) at a mol ratio of 1:1, 0.75:1, 05:1 and 0.25:1 to SNAC, from tablet formulations containing SNAC, SSG and Histidine (His) at a mol ratio of 1:1 to SNAC, and from tablet formulations containing SNAC and SSG.

FIGS. 13A-B present comparative plots showing the SNAC soluble fraction percentage over a 30-minute period incubation at 37° C. in an acidic solution featuring pH of 1.6 (+/−0.1), from tablet formulations containing SNAC, SSG, sodium octanoate (C8) at a mol ratio of 1:1 to SNAC, and histidine at a mol ratio of 1:1 to SNAC,; SNAC, SSG and His at a 1:1 mol ratio to SNAC; SNAC, SSG and C8 at a mol ratio of 1:1 to SNAC; and SNAC and SSG (FIG. 13A), and from tablet formulations containing SNAC, SSG, Na₂CO₃ at a mol ratio of 0.25:1 to SNAC and Arg at a mol ratio of 0.25:1 to SNAC; SNAC, SSG and Na₂CO₃ at a mol ratio of 0.25:1 to SNAC; SNAC, SSG and Arg at a mol ratio of 0.25:1 to SNAC; and SNAC and SSG (FIG. 13B).

### DESCRIPTION OF SPECIFIC EMBODIMENTS OF THE INVENTION

The present invention, in some embodiments thereof, relates to drug delivery, and more particularly, but not exclusively, to formulations and/or systems for oral administration of therapeutically active agents, such as, but not limited to, peptides and/or protein pharmaceuticals.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

While investigating the enhancement of absorption of therapeutically active agents by an exemplary absorption enhancer such as SNAC (sodium 8-N-(2-hydroxybenzoyl)aminocaprylate), the present inventors have uncovered that the ability of such absorption enhancers to enhance absorption (e.g., as determined by bioavailability and/or Cmax) of therapeutically active agents is adversely affected by the acidic environment of the stomach. The inventors have conceived using basic (alkaline) compounds to enhance the performance of absorption enhancers upon oral administration by neutralizing stomach acid, and uncovered that polymers comprising alkaline groups such as carboxylate are particularly effective at protecting the absorption enhancer from stomach acid, as well as enhancing bioavailability of a therapeutically active agent in compositions comprising the absorption enhancer and the therapeutically active agent.

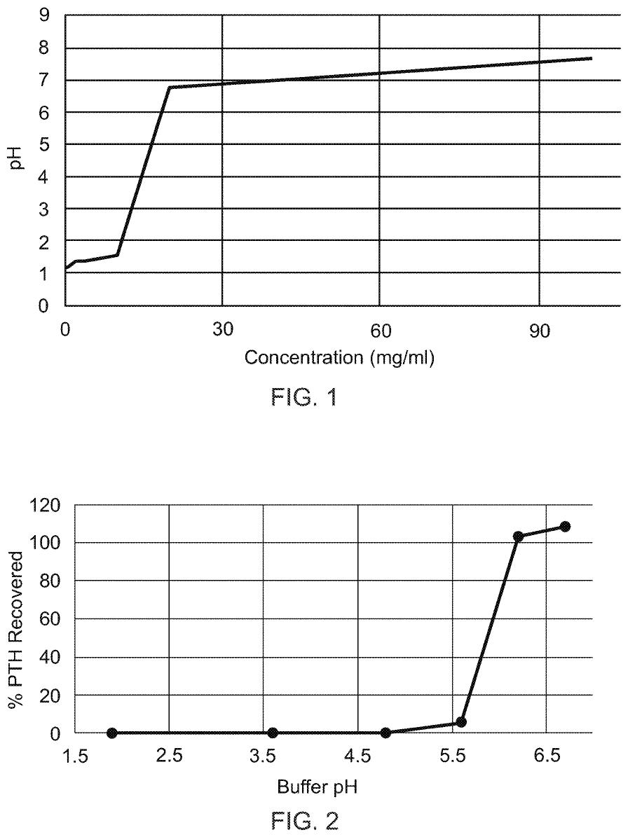

The present inventors have shown that exemplary polymers comprising carboxylate groups produce a local increase in pH in simulated gastric fluid (which increase is capable of reducing protonation of absorption enhancers such as SNAC), and considerably enhance absorption of a therapeutically active agent as compared to corresponding compositions with the same absorption enhancer but without the polymer. See, FIGS. 1-9.

While further studying the effect of acid-neutralizing polymers, the present inventors have uncovered that utilizing these polymers in combination with non-polymeric (small molecule) proton-accepting agents such as antacids (e.g., sodium carbonate, potassium carbonate), amino acids featuring pKa higher than 4, and salts of weak acids such as fatty acid salts, enhance the effect exhibited by these polymers in protecting the absorption enhancer from stomach acid, and thereby enhance the bioavailability of the therapeutically active agent in compositions that comprise the absorption enhancer and the therapeutically active agent (along with the acid-neutralizing polymers and non-polymeric substances).

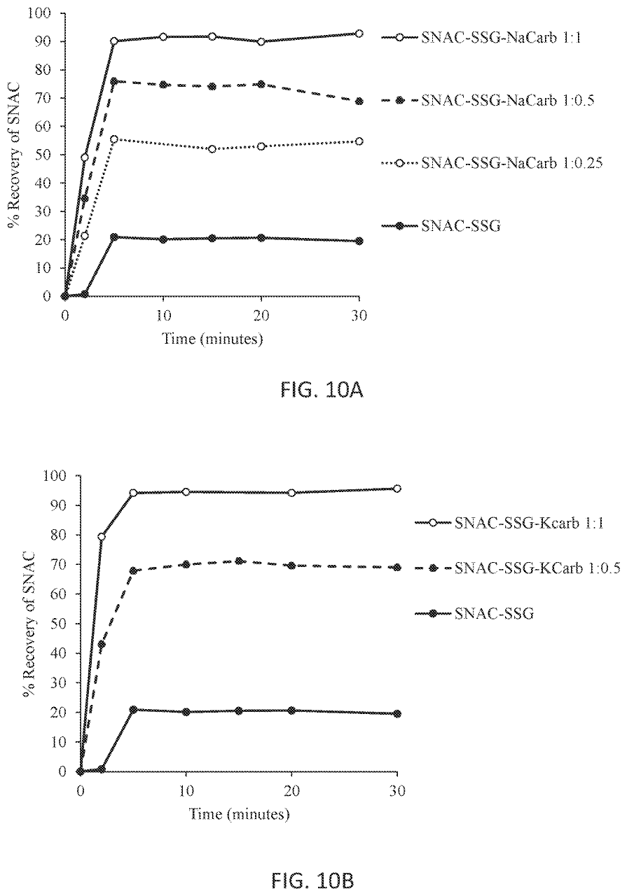

FIGS. 10A-10B, 11, 12 and 13A-B show that exemplary small (non-polymeric) proton-accepting agents, and combinations thereof, considerably enhance the percentage soluble fraction of SNAC over a 30-minute incubation in acidic environment from formulations containing SNAC and SSG. The effect of exemplary small (non-polymeric) proton-accepting agents, when added to formulations containing SNAC and SSG, in enhancing Cmax and AUClast (bioavailability) of exemplary therapeutically active agents was further substantiated in ongoing in vivo studies.

According to an aspect of some embodiments of the invention, there is provided a pharmaceutical composition comprising a therapeutically active agent, an absorption enhancer, a polymer comprising a plurality of alkaline groups (which for brevity is also referred to herein as an “alkaline group-containing polymer” or “alkaline polymer” or “basic polymer” or simply “polymer”), and a non-polymeric (small molecule) proton-accepting agent. In some such embodiments, the composition is in a form of a unit dosage form, for example, a tablet or a combination of more than one tablet (e.g., minitablets).

As explained in detail hereinbelow, the alkaline-group containing polymer is also referred to herein as an acid-neutralizing polymer or as an acid-neutralizing polymeric compound or substance.

As further explained in detail hereinbelow, the non-polymeric (small molecule) proton-accepting agent is also referred to herein as a non-polymeric acid-neutralizing substance, or as a non-polymeric acid-neutralizing agent, or as a non-polymeric acid-neutralizing compound, or, interchangeably, as small molecule proton-accepting agent, or non-polymeric proton-accepting agent or substance, or is abbreviated as sPAA or simply as PAA.

As used herein, the singular form “a”, “an” and “the” include plural references unless the context clearly dictates otherwise. For example, the term “a polymer” or “at least one polymer” may include a plurality of polymers, including mixtures thereof. For example, the term “a proton-accepting agent” or “at least one proton-accepting agent” may include a plurality of substances of compounds or agents, including mixtures thereof.

The term “unit dosage form”, as used herein, describes physically discrete units, each unit containing a predetermined quantity of one or more active ingredient(s) calculated to produce the desired therapeutic effect, in association with at least one pharmaceutically acceptable carrier, diluent, excipient, or combination thereof. Examples of a suitable unit dosage form include, without limitation, a tablet, a capsule, a lozenge, a dragee, a wafer, a sachet, a patch, an ampoule, a vial, a loaded syringe, and a dose of a metered-dose dispenser. Each unit may optionally comprise discrete subunits (e.g., wherein each subunit is in a form described herein, such as a tablet, capsule, lozenge, dragee, etc.), which may be bound to one another or separate from one another.

For example, a unit dosage form according to embodiments of the invention which comprises multiple subunits may optionally be in a form of a unit dosage form comprising discrete subunits bound to one another by a coating and/or matrix, or in a form of a set of dosage forms in a kit (e.g., individually packaged sets of discrete dosage forms), as described, for example, in International Patent Application Publication No. WO 2018/033927, which is incorporated herein by reference as if fully set forth herein.

The pharmaceutical composition and/or unit dosage form according to any of the respective embodiments described herein is preferably formulated to be suitable for oral administration, e.g., as described in more detail herein. Examples of a unit dosage form formulated for oral administration include, without limitation, a tablet, a capsule, a lozenge, a dragee, a wafer, a sachet, an ampoule, and a vial. In exemplary embodiments, the pharmaceutical composition and/or unit dosage form according to any of the respective embodiments described herein is formulated as a tablet or as a plurality of minitablets. In exemplary embodiments, the pharmaceutical composition and/or unit dosage form according to any of the respective embodiments described herein is devoid of a gastroenteric coating, or otherwise comprises a coating that is dissolvable in gastric fluid and/or immediately (e.g., within less than 5 minutes, less than 2 minutes, or less than 1 minute) releases the composition's content upon contacting gastric fluid.

Herein, the phrases “oral administration”, “orally administering” and the like refer to any administration via the mouth, preferably by oral ingestion, such as by swallowing (e.g., as opposed to buccal administration).

In some of any of the embodiments described herein, the composition is formulated to allow absorption of the therapeutically active agent in the stomach, for example, by being devoid of an enteric coating, i.e., a coating which prevents release of an agent and dissolves only in the intestines.

### Alkaline Group-Containing Polymer:

Herein, the term “polymer” refers a compound having at least 4 repeating (backbone) units (and more preferably at least 10 repeating units, for example, from 4 to 1,000, or from 10 to 1,000 repeating units, with higher numbers of repeating units being also contemplated), the repeating units being identical or similar. The term “polymer” encompasses also a co-polymer, which comprises two or more types of repeating units as described herein, for example, at least 4, or preferably at least 10, repeating units of one type, and at least 4, or preferably at least 10, repeating units of another, different type. The units in a co-polymer can be arranged in any order. Polymers and/or co-polymers as described herein are also referred to herein as “polymeric substances”.

The backbone (repeating) units composing a polymer or copolymer as described herein are also referred to herein interchangeably as monomeric units or simply as monomers.

The alkaline group-containing polymer comprises a plurality (i.e., at least 2) of alkaline groups per molecule. In some of any of the embodiments described herein, the alkaline group-containing polymer comprises at least 4 alkaline groups, or at least 10 alkaline groups, or at least 25 alkaline groups, or at least 50 alkaline groups, or at least 100 alkaline groups, on average, per molecule. In some of any of the embodiments described herein, an average number of the alkaline groups per polymer molecule ranges from about 4 to about 1,000, or from about 10 to about 1,000, or from about 25 to about 1,000, or from about 50 to about 1,000, or from about 100 to about 1,000, with higher upper limit also contemplated for each of these ranges.

In some of any of the embodiments described herein, a mean molecular weight of the alkaline group-containing polymer is at least 1 kDa, or at least 2 kDa, or at least 3 kDa, or at least 5 kDa, or at least 10 kDa. In some of any of the embodiments described herein, a mean molecular weight of the alkaline group-containing polymer ranges from about 1 kDa, or from about 2 kDa, or from about 3 kDa, or from about 5 kDa, or from about 10 kDa, or from about 100 kDa, or from about 200 kDa, or from about 300 kDa, or from about 400 kDa, or from about 500 kDa, or from about 600 kDa, or from about 700 kDa, or from about 800 kDa, or from about 1,000 kDa, or from about 2,000 kDa, and up to about 10,000 kDa or higher, including any intermediate values and subranges therebetween.

Herein, the term “alkaline group” encompasses any functional group capable of accepting a proton in aqueous solution, for example, carboxylate or amine groups. It will be appreciated that an alkaline group may be converted to an acidic group (by protonation), and vice versa (by deprotonation), upon contact with a liquid with an appropriate acidity or alkalinity, as these terms are used in the art (e.g., according to pH).

In some of any one of the respective embodiments described herein, the polymer is characterized by a pKa which is at least 1.2, optionally in a range of from 1.2 to 7.5, and optionally in a range of from 1.2 to 5.5 (including any intermediate values and subranges therebetween); for example, from 1.2 to 3 or from 2.5 to 3.5 or from 3 to 4 or from 3.5 to 4.5 or from 4 to 5 or from 4.5 to 5.5, including any intermediate values and subranges therebetween. In some such embodiments, the pKa is at least 2.5, or at least 3, or at least 3.5; for example, from 2.5 to 5.5, or from 3 to 5.5, or from 3.5 to 5.5, or from 4 to 5.5, or from 4.5 to 5.5, including any intermediate values and subranges therebetween.

Herein, the “pKa” of a polymer refers to the pH in an aqueous solution of 0.1 M NaCl, at which 50% of the acid/base (e.g., carboxylate) groups are protonated at 25° C.

Carboxylate groups are exemplary alkaline groups according to some embodiments of the invention.

Herein, the term “carboxylate” refers to—C(═O) O″ groups (i.e., a carboxylate anion), and does not encompass carboxylic acid groups (—C(═O) OH) unless specifically indicated. It will be appreciated that carboxylate groups may be converted to carboxylic acid groups, and vice versa, upon contact with a liquid with an appropriate acidity or alkalinity. For simplicity, some carboxylate group-containing compounds and functional groups described herein (e.g., carboxymethyl, polyacrylic acid) are named as if they were in the carboxylic acid form, but are to be understood as comprising carboxylate groups unless otherwise indicated.

Carboxylate groups according to any of the respective embodiments described herein may optionally be accompanied by a counter-ion to form a salt, for example, a pharmaceutically acceptable salt. A salt (pharmaceutically acceptable salt) of a compound as described herein can alternatively be formed during the synthesis of the compound, e.g., in the course of isolating the compound from a reaction mixture, or re-crystallizing the compound.

Examples of suitable counter-ions which may be comprised by a salt described herein include, without limitation, ammonium, guanidinium, lithium, sodium, potassium, calcium, and magnesium. Sodium is an exemplary counter-ion.

Examples of suitable carboxylate group-containing polymers include, without limitation, salts of alginate, polyacrylic acid, polymethacrylic acid and copolymers of acrylic acid and/or methacrylic acid, and carboxylate group-containing polysaccharide derivatives (e.g., derivatives obtained by oxidation or substitution by a carboxylate-containing group, such as carboxymethyl).

A carboxylate may optionally be comprised by a repeating monomer (backbone unit) which comprises carboxylate, such as a urinate form of a saccharide unit (e.g., mannuronate and guluronate monomers in alginate) and acrylate (e.g., as in a polyacrylic acid salt); comprised by a substituent (e.g., a carboxymethyl substituent, which may optionally be formed by reaction of a polymer with chloroacetic acid) attached to at least a portion of the monomers (e.g., as in carboxymethyl cellulose and sodium starch glycolate); and/or formed by oxidation of at least a portion of the monomers (backbone units), for example, by breaking carbon-carbon bonds and/or by oxidation of a primary carbon (e.g., as in oxidized starch). In some exemplary embodiments, the polymer comprises carboxymethyl groups attached to oxygen atoms.

An amine may optionally be comprised by a repeating monomer (backbone unit) which comprises the amine group; for example, an amino sugar such as a 2-amino-2-deoxysugar (e.g., glucosamine in chitosan), and alkylene imine (e.g., ethylene imine) residues (e.g., as in a polyethyleneimine); and/or comprised by a substituent.

The alkaline group-containing polymer may optionally contain monomers (backbone units) which do not comprise an alkaline group (e.g., acrylate esters, methacrylate esters, acrylamides and methacrylamides), for example, in a copolymer with acrylic acid or methacrylic acid monomers; and/or substituents which do not comprise an alkaline group (e.g., substituted or non-substituted alkyl groups such as methyl, ethyl, hydroxyethyl and hydroxypropyl), for example, in a polymer (e.g., polysaccharide) also substituted by an alkaline group-containing substituent.

The alkaline groups, upon oral administration, are such that are capable of neutralizing an acid by undergoing protonation, to form an acidic group (e.g., a carboxylic acid group or ammonium group), thus increasing the pH of a solution that is in contact with the respective alkaline group-containing polymer.

Without being bound by any particular theory, it is believed that the acid-neutralizing polymer described herein is more suitable than conventional antacids, as the polymer is a large molecule which does not readily diffuse throughout the stomach (thus minimizing dilution of the polymer), which may result in a longer period of efficacy. It is further believed that the polymer described herein provides an additional advantageous effect (which is generally lacking in conventional antacids) of forming a viscous medium which limits influx of stomach acid to the absorption enhancer, thus further reducing inactivation of absorption enhancer by stomach acid; and/or limits diffusion of the absorption enhancer and/or therapeutically active agent throughout the stomach, thus enhancing the effect of the absorption enhancer by maintaining a higher local concentration of absorption enhancer in the vicinity of the therapeutically active agent. In this manner the composition may facilitate absorption of the therapeutically effective agent for a longer time (e.g., by reducing the rate of inactivation of absorption enhancer by protonation and/or by dilution) and/or increase the peak rate of absorption of the therapeutically effective agent (e.g., by providing a higher local concentration of absorption enhancer in active form, as discussed herein). It is further believed that such formation of a viscous medium may facilitate adhesion of the composition to the stomach wall (e.g., by increasing contact area between the soft medium and the stomach wall), thereby promoting absorption via stomach tissue.

In some embodiments of any one of the embodiments described herein, the alkaline group-containing polymer swells upon contact with water (and is also referred to herein in the context of these embodiments as “water-swellable”). In some such embodiments, the polymer is not water-soluble.

Herein, the phrase “swells upon contact with water” refers to an ability of a substance to absorb at least its own weight in water upon contact with water (e.g., pure water) at 37° C. (that is, the substance has a swelling capacity of at least 100%), and optionally at least twice its own weight (a swelling capacity of at least 200%) or at least 5-fold its own weight (a swelling capacity of at least 500%) or at least 10-fold its own weight or at least 20-fold its own weight in water.

By “swelling capacity” it is meant that the material is capable of swelling the indicated weight percentage water, of its weight before swelling. The swelling capacity (Qt) can be calculated using the formula:

$$Qt = ( Ws - Wd ) / Ws * 100 \% .$$

wherein Ws is the weight after swelling and Wd is the dry weight (weight before swelling).

Herein, the term “water-soluble” refers to a compound having a solubility of at least 1 gram per liter in water (e.g., pure water) at 37° C.

Herein, the term “water-insoluble” refers to a compound having a solubility of less than 1 gram per liter in water (e.g., pure water) at 37° C.

A typical assay for quantitatively determining solubility of a substance is the “shake-flask method”, in which an excess of the tested substance added to a volume (e.g., 100 ml) of solvent (e.g., water) in a container (e.g., flask or vial) and shaken under predetermined conditions (e.g., a temperature of 37° C.) so as to achieve thermodynamic equilibrium. Residual solid is then removed (e.g., by filtration and/or centrifugation), and the concentration of dissolved substance is determined by a technique such as HPLC. The concentration may optionally be determined at different time points in order to ascertain that equilibrium has been reached.

Without being bound by any particular theory, it is believed that swelling upon contact with water (e.g., in gastric medium) is advantageous as it enhances penetration of water and maximizes contact between the surrounding solution and alkaline groups upon oral administration. For example, a polymer with a lower degree of penetration of water (e.g., into granules of the polymer) may exhibit slower than optimal neutralization of stomach acid, even if the amount of alkaline groups is high; for example, due to less effective diffusion of acid to the alkaline groups. It is further believed that water solubility, although also effective at maximizing contact between the surrounding solution and alkaline groups, is less desirable than swelling, as dissolution of the polymer disadvantageously results in a less localized effect (e.g., similar to conventional antacids discussed herein).

In some of any of the embodiments described herein, the polymer is a crosslinked polymer.

The polymer can be cross-linked via covalent bonds, by means of cross-linking moieties that are each being covalently attached to, and thereby connects, two or more backbone units in the polymer, and/or via electrostatic bonds, by means of cross-linking moieties (e.g., ions such as cations) that are each being electrostatically attached to, and thereby connects, two or more backbone units in the polymer. The number of cross-linking moieties determines the degree of cross-linking of a cross-linked polymer.

Without being bound by any particular theory, it is believed that crosslinking is advantageous by minimizing dissolution of the polymer (e.g., in saliva and/or stomach acid) which could result in “wastage” of the polymer; and/or by enhancing water-absorption (e.g., by disrupting binding between different polymer chains, thereby facilitating entry of water molecules between the chains). The degree of crosslinking may affect swelling capacity; for example, insufficient crosslinking may be associated with dissolution (as opposed to swelling), and/or to reduced swelling and/or reduced penetration of water. However, excessive crosslinking may reduce swelling capacity by limiting the ability of polymer to expand and provide space for water to enter. The degree of crosslinking should not affect the acid-neutralizing capacity of the polymer. The degree of cross-linking can be selected or pre-determined for each selected polymer, in accordance with its properties, so as to provide the desired hydration level.

In some of any of the embodiments described herein, the polymer comprises a polysaccharide, for example, a crosslinked polysaccharide. The polymer is optionally composed primarily (i.e., more than 50 percent by weight) of glucose units, which may optionally be linked, by glycosidic bonds such as α(1→4) and/or α(1→6) glycosidic bonds (e.g., as in starch) and/or β(1→4) glycosidic bonds (e.g., as in cellulose).

The term “monosaccharide”, as used herein and is well known in the art, refers to a simple form of a sugar that consists of a single saccharide molecule which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms.

Monosaccharides are the building blocks of polysaccharides (such as cellulose and starch).

The term “polysaccharide” as used herein refers to a compound that comprises 10 or more monosaccharide units, as these are defined herein, linked to one another via a glycosyl bond (—O—). The glycosyl bonds between saccharide units in a polysaccharide can all be the same, or can include two or more types of glycosyl bonds, for example, be such that glucose units are linked via α(1→4) and/or α(1→6) glycosidic bonds.

Herein, the term “starch” encompasses polysaccharides composed of amylose (glucose units linked via α(1→4) glycosidic bonds), and amylopectin (glucose units linked via α(1→4) and α(1→6) glycosidic bonds). Different types of starch differ from one another by the ratio between amylose and amylopectin, which typically depends on the source of the starch.

In some of any of the embodiments relating to a polysaccharide, the polysaccharide is a starch derivative and/or a cellulose derivative, that is, starch and/or cellulose (e.g., crosslinked starch and/or crosslinked cellulose) derivatized so as to comprise alkaline (e.g., carboxylate) groups, for example, by being substituted by carboxymethyl groups (e.g., wherein oxygen atoms thereof are attached to carboxymethyl groups). Sodium starch glycolate is an exemplary starch derivative. Croscarmellose sodium is an exemplary cellulose derivative.

Herein, “sodium starch glycolate” refers to any sodium salt of starch (as defined herein) substituted by carboxymethyl groups, and may be crosslinked or non-crosslinked. In some of any of the respective embodiments, the sodium starch glycolate is crosslinked covalently by phosphate groups (—O—P(═O)(—O⁻)—O—), each linking two (or more) saccharide (glucose) units.

Herein, “croscarmellose sodium” refers to any sodium salt of cellulose (as defined herein) substituted by carboxymethyl groups, and crosslinked covalently by ester bonds between carboxymethyl groups and a saccharide unit.

The carboxymethyl groups (or any other alkaline group(s)) can be attached to one or more of the hydroxy substituents of one or more saccharide (e.g., glucose) units composing the polysaccharide, so as to form a —O═CH₂—C(═O)—O⁻ alkaline group which substitute the monosaccharide at one or more positions. In some embodiments, the carboxymethyl groups (or any other alkaline group(s)) are attached at least to the hydroxymethyl group of one or more glucose units that compose the polysaccharide. In some embodiments, the carboxymethyl groups (or any other alkaline group(s)) are attached to at least 10%, or at least 20%, or at least 30%, or at least 40%, or at least 50%, or at least 60%, or at least 70%, or at least 80%, or at least 90% or about all of the saccharide units composing the polysaccharide. In some of these embodiments, one carboxymethyl group (or any other alkaline group) is attached to a saccharide unit comprising same; and in some of these embodiments, the carboxymethyl group (or any other alkaline group) is attached to the hydroxymethyl substituent of the glucose.

In some of any of the embodiments described herein, a concentration of the polymer comprising a plurality of alkaline groups (according to any of the respective embodiments described herein) in the composition is at least 5 weight percent. In some such embodiments, the concentration of the polymer is at least 10 weight percent. In some such embodiments, the concentration of the polymer is at least 15 weight percent. In some embodiments, the concentration of the polymer is at least 20 weight percent. In some embodiments, the concentration of the polymer is at least 25 weight percent. In some embodiments, the concentration of the polymer is at least 30 weight percent. In some embodiments, the concentration of the polymer is at least 35 weight percent. In some embodiments, the concentration of the polymer is at least 40 weight percent. In some embodiments, the concentration of the polymer is at least 50 weight percent. In some embodiments, the concentration of the polymer is at least 60 weight percent. In some embodiments, the concentration of the polymer is at least 70 weight percent. In some embodiments, the concentration of the polymer is at least 80 weight percent. In some embodiments, the concentration of the polymer is at least 90 weight percent.

By “weight percent” (or w/w %, or % wt.) it is meant the weight of the indicated substance out of the total weight of a composition comprising same.

In some of any of the embodiments described herein, a concentration of the polymer comprising a plurality of alkaline groups (according to any of the respective embodiments described herein) in the composition ranges from 5 to 90 weight percent, of the total weight of the composition, or from 5 to 80, or from 5 to 70, or from 5 to 50, or from 5 to 40, or from 5 to 30, or from 10 to 90, or from 10 to 80, or from 10 to 70, or from 10 to 60, or from 10 to 50, or from 10 to 40, or from 10 to 30, or from 10 to 90, or from 10 to 80, or from 10 to 70, or from 10 to 60, or from 10 to 50, or from 10 to 40, or from 10 to 30, or from 30 to 90, or from 30 to 80, or from 30 to 70, or from 30 to 60, or from 30 to 50, or from 30 to 40, or from 40 to 90, or from 40 to 80, or from 40 to 70, or from 40 to 60, or from 40 to 50, or from 50 to 90, or from 50 to 80, or from 50 to 70, or from 50 to 60, or from 60 to 90, or from 60 to 80, or from 60 to 70, or from 80 to 90, or from 70 to 80, or from 80 to 90, weight percent, of the total weight of the composition, including any intermediate values and subranges therebetween.

In some of any of the embodiments described herein, a concentration of the polymer comprising a plurality of alkaline groups (according to any of the respective embodiments described herein) in the composition is at least 5, or at least 10, or at least 20, weight percent, of the total weight of the composition.

In some of any of the embodiments described herein, a concentration of the polymer comprising a plurality of alkaline groups (according to any of the respective embodiments described herein) in the composition ranges from 5 to 50 weight percent, of the total weight of the composition, or from 5 to 30, or from 5 to 20, or from 10 to 50, or from 10 to 40, or from 10 to 30, or from 10 to 15, weight percent, of the total weight of the composition, including any intermediate values and subranges therebetween.

In some of any of the embodiments described herein, a concentration of the polymer comprising a plurality of alkaline groups (according to any of the respective embodiments described herein) in the composition ranges from 10 to 20 or from 10 to 15, weight percent, of the total weight of the composition, including any intermediate values and subranges therebetween, for therapeutically active agents featuring plasma half-life (T1/2 when administered intravenously (IV)) lower than 2 hours, or lower than 90 minutes, or lower than 60 minutes, or lower than 30 minutes.

In some of any of the embodiments described herein, a concentration of the polymer comprising a plurality of alkaline groups (according to any of the respective embodiments described herein) in the composition ranges from 15 to 30 or from 20 to 30, or from 20 to 25, weight percent, of the total weight of the composition, including any intermediate values and subranges therebetween, for therapeutically active agents featuring plasma half-life (T1/2, when administered intravenously (IV)) higher than 30 minutes, or higher than 60 minutes, or higher than 90 minutes, or higher than 2 hours.

In some of any of the embodiments described herein, a weight ratio of the polymer comprising a plurality of alkaline groups (according to any of the respective embodiments described herein) and the absorption enhancer (according to any of the respective embodiments described herein) in the composition ranges from 1:1 to 1:10, or from 1:2 to 1:10, or from 1:1 to 1:8, or from 1:2 to 1:8, or from 1:1 1:6, or from 1:2 to 1:6, or from 1:1 to 1:5, or from 1:2 to 1:5, or from 1:1 to 1:4, or from 1:2 to 1:4, or from 1:3 to 1:10, or from 1:3 to 1:8, or from 1:3 to 1:6, or from 1:3 to 1:5, including any intermediate values and subranges therebetween.

In some of any of the embodiments described herein, a total amount (concentration) of the polymer and the non-polymeric proton-accepting agent in the composition ranges from 5 to 90 weight percent, of the total weight of the composition, or from 5 to 80, or from 5 to 70, or from 5 to 50, or from 5 to 40, or from 5 to 30, or from 10 to 90, or from 10 to 80, or from 10 to 70, or from to 50, or from 10 to 40, or from 10 to 30, or from 20 to 90, or from 20 to 80, or from 20 to 70, 10 or from 20 to 60, or from 20 to 50, or from 20 to 40, or from 20 to 30, or from 30 to 90, or from 30 to 80, or from 30 to 70, or from 30 to 60, or from 30 to 50, or from 30 to 40, or from 40 to 90, or from 40 to 80, or from 40 to 70, or from 40 to 60, or from 40 to 50, or from 50 to 90, or from 50 to 80, or from 50 to 70, or from 50 to 60, or from 60 to 90, or from 60 to 80, or from 60 to 70, or from 80 to 90, or from 70 to 80, or from 80 to 90, weight percent, of the total weight of the composition, including any intermediate values and subranges therebetween. In some of any of the embodiments described herein, a total amount (concentration) of the polymer and the non-polymeric proton-accepting agent in the composition ranges from 5 to 60 weight percent, of the total weight of the composition, or from 5 to 50, or from 5 to 40, or from 5 to 30, or from 5 to 20, or from 10 to 60, or from 10 to 50, or from 10 to 40, or from 10 to 30, weight percent, of the total weight of the composition, including any intermediate values and subranges therebetween. In some of any of the embodiments described herein, a total amount (concentration) of the polymer and the non-polymeric proton-accepting agent in the composition ranges from 10 to 60 weight percent, of the total weight of the composition, or from 10 to 50, or from 10 to 40, or from 10 to 30, or from 20 to 60 weight percent, of the total weight of the composition, or from 20 to 50, or from 20 to 40, weight percent, of the total weight of the composition, including any intermediate values and subranges therebetween.

Without being bound by any particular theory, it is believed that a relatively high concentration of water-absorbing (e.g., water-swellable) polymers such as sodium starch glycolate and croscarmellose sodium (e.g., relative to compositions which utilize such polymers as disintegrants) is associated with generation of a viscous medium upon contact with an aqueous solution (e.g., stomach acid) which limits diffusion in the vicinity of the composition, which is significantly different than their effect (e.g., promotion of disintegration of a solid composition) at lower concentrations.

Compositions which utilize such polymers as disintegrants further differ from compositions according to some embodiments of the invention in that disintegrants are normally used in compositions which also comprise a large proportion of insoluble components such as microcrystalline cellulose (e.g., as opposed to compositions comprising large proportions of water soluble components such as SNAC); for example, because the mechanism of the disintegrant involves applying internal forces to a solid insoluble component and/or because a large proportion of water soluble component(s) renders a composition readily soluble, such that a disintegrant would be redundant.

In some of any of the embodiments relating to a polysaccharide described herein, the polysaccharide is characterized by a degree of substitution which is at least 0.1; for example, in a range of from 0.1 to 2 or from 0.1 to 1.5 or from 0.1 to 1 or from 0.1 to 0.5 or from 0.1 to 0.3 or from 0.1 to 0.2, including any intermediate values and subranges therebetween. In some such embodiments, the degree of substitution is at least 0.2; for example, in a range of from 0.2 to 2 or from 0.2 to 1.5 or from 0.2 to 1 or from 0.2 to 0.5, including any intermediate values and subranges therebetween. In some embodiments, the degree of substitution is at least 0.3; for example, in a range of from 0.3 to 2 or from 0.3 to 1.5 or from 0.3 to 1 or from 0.3 to 0.5, including any intermediate values and subranges therebetween. In some embodiments, the degree of substitution is at least 0.4; for example, in a range of from 0.4 to 2 or from 0.4 to 1.5 or from 0.4 to 1, including any intermediate values and subranges therebetween.

Herein, “degree of substitution” refers to the ratio of functional groups comprising an alkaline group or a conjugate acid thereof, such as a carboxylate or carboxylic acid group (e.g., a carboxymethyl group), to monomers (backbone units) of a polymer (e.g., saccharide units of a polysaccharide).

In some of any of the respective embodiments described herein, a concentration of alkaline groups of the polymer in the composition is at least 0.03 millimoles per gram composition; for example, from 0.03 to 5 millimoles per gram or from 0.03 to 2 millimoles per gram or from 0.03 to 1 millimoles per gram or from 0.03 to 0.5 millimoles per gram or from 0.03 to 0.2 millimoles per gram, including any intermediate values and subranges therebetween. In some embodiments, the concentration of alkaline groups of the polymer is at least 0.1 millimoles per gram composition; for example, from 0.1 to 5 millimoles per gram or from 0.1 to 2 millimoles per gram or from 0.1 to 1 millimoles per gram or from 0.1 to 0.5 millimoles per gram, including any intermediate values and subranges therebetween. In some embodiments, the concentration of alkaline groups of the polymer is at least 0.2 millimoles per gram composition; for example, from 0.2 to 5 millimoles per gram or from 0.2 to 2 millimoles per gram or from 0.2 to 1 millimoles per gram or from 0.2 to 0.5 millimoles per gram, including any intermediate values and subranges therebetween. In some embodiments, the concentration of alkaline groups of the polymer is at least 0.5 millimoles per gram composition; for example, from 0.5 to 5 millimoles per gram or from 0.5 to 2 millimoles per gram or from 0.5 to 1 millimoles per gram, including any intermediate values and subranges therebetween. In some embodiments, the concentration of alkaline groups of the polymer is at least 1 millimoles per gram composition; for example, from 1 to 5 millimoles per gram or from 1 to 2 millimoles per gram, including any intermediate values and subranges therebetween.

In some of any of the embodiments described herein, the composition is a unit dosage form composition, and an amount of alkaline groups of the polymer in the unit dosage form (which correlates with the acid-neutralizing capacity of the polymer in the unit dosage form) is at least 0.03 millimoles; for example, from 0.03 to 10 millimoles or from 0.03 to 3 millimoles or from 0.03 to 1 millimoles or from 0.03 to 0.3 millimoles, including any intermediate values and subranges therebetween. In some embodiments, the amount of alkaline groups in the unit dosage form is at least 0.1 millimoles; for example, from 0.1 to 10 millimoles or from 0.1 to 3 millimoles or from 0.1 to 1 millimoles, including any intermediate values and subranges therebetween. In some embodiments, the amount of alkaline groups in the unit dosage form is at least 0.3 millimoles; for example, from 0.3 to 10 millimoles or from 0.3 to 3 millimoles, including any intermediate values and subranges therebetween. In some embodiments, the amount of alkaline groups in the unit dosage form is at least 1 millimoles; for example, from 1 to 10 millimoles, including any intermediate values and subranges therebetween.

As mentioned hereinabove, the alkaline group-containing polymer according to any of the respective embodiments described herein may optionally comprise two or more (e.g., at least 3, or at least 4) distinct polymers (e.g., in admixture) which comprise alkaline groups. Thus, for example, sodium starch glycolate may represent a portion of all the alkaline group-containing polymer, e.g., at least 5 weight percent or at least 10 weight percent or at least 20 weight percent or at least 30 weight percent or at least 40 weight percent or at least 50 weight percent or at least 60 weight percent or at least 70 weight percent or at least 80 weight percent or at least 90 weight percent of the alkaline group-containing polymer (according to any of the respective embodiments described herein), the remainder being one or more alkaline group-containing polymers other than sodium starch glycolate. Alternatively or additionally, croscarmellose sodium may represent a portion of all the alkaline group-containing polymer, e.g., at least 5 weight percent or at least 10 weight percent or at least 20 weight percent or at least 30 weight percent or at least 40 weight percent or at least 50 weight percent or at least 60 weight percent or at least 70 weight percent or at least 80 weight percent or at least 90 weight percent of the alkaline group-containing polymer (according to any of the respective embodiments described herein), the remainder being one or more alkaline group-containing polymers other than croscarmellose sodium.

It is noted that polymers in general typically comprise a population of molecules of different sizes and with slightly different geometries (e.g., branching patterns and/or sequence of monomers in a copolymer). In accordance with standard practice in the art, such a population of molecules is not considered to represent “distinct polymers”. Rather, “distinct polymers” refers to chemical differences, such as polymers composed of different monomers (backbone units) and/or crosslinkers and/or of different degree of cross-linking.

The effect of the polymer on absorption of the therapeutically active agent may optionally be determined by comparing absorption upon oral administration of a composition according to embodiments of the invention with a corresponding composition comprising the same amount of other ingredients other than the alkaline group-containing polymer (e.g., when the alkaline group-containing polymer is no more than 50 weight percent of the composition according to embodiments of the invention), such that the total mass is lower (due to the absence of alkaline group-containing polymer). In some such embodiments, both compositions are in a form of a tablet, and the tablets have the same cross-section (e.g., diameter of a circular cross-section), and the tablet with a lower weight is thinner in the axis perpendicular to the cross-section of the tablet.

Alternatively, the effect of the polymer on absorption of the therapeutically active agent may optionally be determined by comparing absorption upon oral administration of a composition according to embodiments of the invention with a corresponding composition comprising absorption enhancer instead of the alkaline group-containing polymer (e.g., the corresponding composition being identical in all aspects except for the presence of additional absorption enhancer instead of the polymer), such that the total mass of the compositions is the same (e.g., when the alkaline group-containing polymer is more than 50 weight percent of the composition according to embodiments of the invention).

In some embodiments of any one of the embodiments described herein, a Cmax and/or bioavailability of the composition (i.e., of the therapeutically active agent in the composition) upon oral administration is at least 20% higher than (120% of the level of) a Cmax and/or bioavailability of a corresponding composition without the polymeric and non-polymeric acid neutralizing substances (the alkaline group-containing polymer and the proton accepting agent, as described herein in any of the respective embodiments). In some embodiments, the Cmax and/or bioavailability is at least 50% higher than (150% of the level of) the Cmax and/or bioavailability upon oral administration of a corresponding composition without the alkaline group-containing polymer and the proton accepting agent. In some embodiments, the Cmax and/or bioavailability is at least twice (200% of the level of) the Cmax and/or bioavailability upon oral administration of a corresponding composition without the alkaline group-containing polymer and the proton accepting agent. In some embodiments, the Cmax and/or bioavailability is at least four-fold (400% of the level of) the Cmax and/or bioavailability upon oral administration of a corresponding composition without the alkaline group-containing polymer and the proton accepting agent. In some embodiments, the Cmax and/or bioavailability is at least six-fold (600% of the level of) the Cmax and/or bioavailability upon oral administration of a corresponding composition without the alkaline group-containing polymer and the proton accepting agent. In some embodiments, the Cmax and/or bioavailability is at least ten-fold (1000% of the level of) the Cmax and/or bioavailability upon oral administration of a corresponding composition without the alkaline group-containing polymer and the proton accepting agent.

Cmax and/or bioavailability may optionally be determined by administering the composition orally to subjects (e.g., human subjects) and determining a level of the therapeutically active agent in the blood at frequent intervals by taking blood samples.

Bioavailability may be determined by comparing a ratio of an area under the curve upon oral administration (e.g., over the course of 12 or 24 hours) to an area under curve over the same time period for intravenously administered therapeutically active agent, using standard techniques (e.g., data processing algorithms) known in the art. The therapeutically active agent may be injected at a lower dose (e.g., for safety reasons) and the areas under curve normalized to the total amount administered.

### Non-Polymeric Proton-Accepting Agent:

Herein throughout, the phrase “non-polymeric proton-accepting agent”, is also referred to herein interchangeably as “non-polymeric acid-neutralizing agent” or as “small molecule proton accepting agent” or as “proton-accepting small molecule agent” or as “small proton-accepting agent” or simply as “proton-accepting agent” or abbreviated as “PAA” or “sPAA”.

Herein throughout the phrase “non-polymeric proton-accepting agent” and related phrases describe a pharmaceutically acceptable compound featuring a pKa higher than the gastric pH, by at least 1 pH unit, and thus, as being at least 2, preferably at least 3, more preferably at least 5, more preferably at least 6, and more preferably higher than 7 pH units than the gastric pH.

In some embodiments, the non-polymeric proton-accepting agent features a pKa higher than the pKa of the absorption enhancer, by at least 0.5, or at least unit. For example, when an absorption enhancer is a salt of NAC, which features pKa of about 5, the PAA features pKa of at least 5.5, preferably at least 6.

In some of any one of the respective embodiments described herein, the proton-accepting agent has a pKa of at least 2.0, optionally in a range of 2.0 to 14.0, and optionally in a range of 6.0 to 11.0 (including any intermediate values and subranges therebetween); for example, from 6.0 to 14.0, or from 6.0 to 13.0, or from 6.0 to 12.0, or from 6.0 to 11.0, or from 6.0 to 10.0, or from 6.0 to 9.0, or from 6.0 to 8.0, or from 6.0 to 7.0, or from 6.0 to 6.5, including any intermediate values and subranges therebetween. In some such embodiments, the pKa is at least 2.0, or at least 2.5, or at least 3.0, or at least 3.5, or at least 4.0, or at least 4.5, or at least 5.0, or at least 5.5, or at least 6.0, or at least 6.5, or at least 7.0, or at least 7.5, or at least 8.0, or at least 8.5, or at least 9.0, or at least 9.5, or at least 10.0, or at least 10.5, or at least 11.0, or at least 11.5, or at least 12.0, or at least 12.5, or at least 13.0, or at least 14.0, including any intermediate values and subranges therebetween.

In some of any of the embodiments described herein, the proton-accepting agent independently features pKa higher by at least 0.25, or by at least 0.5, or by at least 1, or by at least 2, or by at least 3, or by at least 4, or by at least 5, or by at least 6, or by at least 7, than a pKa of the absorption enhancer.

In some of any of the embodiments described herein, the at least one small proton-accepting agent comprises an amino acid featuring a pKa of at least 4, or at least 5, or at least 6, or at least 7, or at least 8, or at least 9, or at least 10, or at least 11, or at least 12.

Herein, the “pKa” of a non-polymeric proton-accepting agent refers to the pH in an aqueous solution of 0.1 M NaCl, at which 50% of the acid/base (e.g., carboxylate) groups are protonated at 25° C.

Without being bound by any particular theory, it is believed that dissolved SNAC diffuses away from the buffered center of the formulation matrix to the unbuffered, acidic periphery. It is further believed that small proton accepting agent(s), added to a formulation containing an acid-neutralizing polymer and therapeutically active agent, diffuse away from the core of the formulation matrix at the same kinetics as SNAC, thus preventing its precipitation at the periphery of the formulation, from where acid-neutralizing polymer(s) are unable to reach.

In some embodiments, the “non-polymeric proton-accepting agent” encompasses such pharmaceutically acceptable compounds which diffuse away from a solid formulation (e.g., as described herein in any of the respective embodiments), upon contacting gastric fluid (when the solid formulation starts to disintegrate and/or release its content), at diffusion kinetics similar to or no more than 50 percent faster or slower than the absorption enhancer (e.g., SNAC).

In some embodiments, the diffusion kinetics correlates to the molecular weight of the PAA and/or to its water solubility.

In some embodiments, the PAA has a molecular weight that is similar to the molecular weight of the absorption enhancer, for example, is no more than 50%, or no more than 20% lower or higher than the molecular weight of the absorption enhancer. For example, when the absorption enhancer is a salt of SNAC such as a sodium salt of NAC, which has a molecular weight of about 301 grams/mol, the PAA has a molecular weight lower than 1,000, preferably lower than 600, or lower than 500 grams/mol, for example, of from 100 to 600, or from 100 to 500, grams/mol, including any intermediate values and subranges therebetween.

In some of any of the embodiments described herein, the at least one small proton-accepting agent has a molecular weight in a range of 20% to about 200%, or from about 20% to about 120%, or from about 20% to about 120%, or from about 5% to 150%, optionally in a range of from 5% to 140%, or from 5% to 130%, or from 5% to 120%, or from 5% to 110%, or from 5% to 100%, or from 5% to 90%, or from 5% to 80%, or from 5% to 70%, or from 5% to 60%, or from 5% to 50%, or from 5% to 40%, or from 5% to 30%, or from 5% to 25%, relative the molecular weight of the absorption enhancer (e.g., is a salt of NAC such as a sodium salt of NAC), including any intermediate values and subranges therebetween.

In some embodiments, the PAA is characterized by water solubility of at least 1 mg/ml, preferably at least 10 mg/ml, at room temperature (e.g., 25° C.).

In some of any of the embodiments described herein, the at least one small proton-accepting agent has a water solubility in a range of 20% to about 200%, or from about 20% to about 120%, or from about 20% to about 120%, or from about 5% to 150%, optionally in a range of from 5% to 140%, or from 5% to 130%, or from 5% to 120%, or from 5% to 110%, or from 5% to 100%, or from 5% to 90%, or from 5% to 80%, or from 5% to 70%, or from 5% to 60%, or from 5% to 50%, or from 5% to 40%, or from 5% to 30%, or from 5% to 25%, relative the water solubility of the absorption enhancer, including any intermediate values and subranges therebetween.

In some embodiments, the PAA has a molecular weight lower than 2,000, or lower than 1,500, or lower than 1,000, preferably lower than 500, grams/mol, for example, in a range of from 100 to 1,000, or from 100 to 800, or from 100 to 600, or from 100 to 300, grams/mol, and water solubility as described herein.

In some embodiments, the PAA features pKa of at least 5.5, has a molecular weight lower than 2,000, or lower than 1,500, or lower than 1,000, preferably lower than 500, grams/mol, for example, in a range of from 100 to 1,000, or from 100 to 800, or from 100 to 600, or from 100 to 300, grams/mol, and water solubility as described herein.

The therapeutically active agent, the absorption enhancer (e.g., SNAC) and the polymer comprising a plurality of alkaline groups, as described herein in any of the respective embodiments, are excluded from the scope of the phrase “small proton-accepting agent”, even though they may exhibit some ability to neutralize stomach acid, in some embodiments of the invention.

Exemplary non-polymeric proton-accepting agents that are suitable for use in the context of the present embodiments include, without limitation, antacid compounds, alkaline amino acids (amino acid residues which are protonated at physiological pH), and fatty acid salts.

Herein throughout, the term “antacid compound” refers to any pharmaceutically acceptable compound capable of neutralizing stomach acid (e.g., HCl in aqueous solution), preferably wherein one mole of antacid compound is capable of neutralizing at least 0.5 mole of HCl, and more preferably capable of neutralizing at least 1 mole of HCl. An “antacid compound” according to some embodiments of the present invention encompasses compounds known as drugs for treating conditions associated with stomach acidity such as heartburn.

In some embodiments, the at least one antacid compound is one or more of calcium carbonate, calcium gluconate, calcium citrate, sodium carbonate, sodium bicarbonate, sodium gluconate, sodium citrate, sodium hydroxide, potassium carbonate, potassium bicarbonate, potassium gluconate, potassium citrate, potassium hydroxide, magnesium carbonate, magnesium gluconate, magnesium citrate, magnesium oxide and magnesium hydroxide.

In some embodiments, the at least one antacid compound is one or more of calcium carbonate, calcium gluconate, calcium citrate, sodium carbonate, sodium bicarbonate, sodium hydroxide, potassium carbonate, potassium bicarbonate, potassium gluconate, potassium citrate, potassium hydroxide, magnesium carbonate, magnesium gluconate, magnesium citrate, magnesium oxide and magnesium hydroxide.

In some embodiments, the at least one antacid compound is one or more of calcium carbonate, calcium gluconate, sodium carbonate, sodium bicarbonate, sodium citrate, sodium hydroxide, potassium carbonate, potassium bicarbonate, potassium citrate, potassium hydroxide, magnesium carbonate, magnesium hydroxide, magnesium oxide, aluminum carbonate, and aluminum hydroxide.

In some embodiments, the at least one antacid compound is one or more of calcium carbonate, calcium citrate, sodium bicarbonate, sodium hydroxide, magnesium carbonate, magnesium citrate, magnesium hydroxide, aluminum carbonate, and aluminum hydroxide.

In some embodiments, the at least one antacid compound is one or more of calcium carbonate, sodium carbonate, sodium bicarbonate, potassium bicarbonate, magnesium carbonate, magnesium hydroxide, and aluminum hydroxide.

Amino acids that suitable for use in the context of the present embodiments include alkaline amino acids, which feature a side chain that can be positively charged in gastric pH, or which features pKa higher than the gastric pH, by at least 1 pH unit, and thus, as being at least 2, preferably at least 3, more preferably at least 5, more preferably at least 6, and more preferably higher than 7 pH units than the gastric pH. Preferably, the amino acid has a side chain that features pKa that is higher by at least 0.5, preferably by at least 1 unit than the pKa of the absorption enhancer. In some embodiments, the alkaline amino acid has a side chain that features pKa of at least 5, or at least 6, for example, of from 5 to 14, or from 6 to 14, or from 5 to 12 or from 6 to 12, or from 5 to 10 or from 6 to 10, including any intermediate values and subranges therebetween.

In some embodiments, the alkaline amino acid features pKa as described herein, and has a MW and hydrophilicity as described herein for a PAA.

The amino acid includes both D- and L-amino acids.

The amino acid can be naturally occurring, amino acids that modified post-translationally in vivo and/or other amino acids that are not naturally-occurring, including, for example, hydroxyproline, phosphothreonine, hydroxylysine, isodesmosine, and ornithine.

In some embodiments, the at least one basic amino acid compound is one or more of L-histidine, D-histidine, D-N-methylhistidine, L-N-methylhistidine, D-α-methylhistidine, L-α-methylhistidine, L-arginine, D-arginine, D-N-methylarginine, L-N-methylarginine, D-α-methylarginine, L-α-methylarginine, L-lysine, D-lysine, D-N-methyllysine, L-N-methyllysine, D-α-methyllysine, and L-α-methyllysine.

In some embodiments, the at least one basic amino acid compound is one or more of D-histidine, L-histidine, L-arginine, D-arginine, L-lysine and D-lysine.

In some embodiments that amino acid is or comprises arginine, e.g., L-arginine, also referred to herein as “Arg”.

Herein and in the art, the phrase “fatty acid salt” describes a compound, or a mixture of compounds, featuring pKa higher than the gastric pH, by at least 1 pH unit, and thus, as being at least 2, preferably at least 3, more preferably at least 5, more preferably at least 6, and more preferably higher than 7 pH units higher than the gastric pH. Preferably, the fatty acid salt features pKa that is higher by at least 0.5 or by at least 1 than the pKa of the absorption enhancer. Preferably, the fatty acid salt features pKa of at least 5, or at least 6, for example, of from 5 to 14, or from 6 to 14, or from 5 to 12 or from 6 to 12, or from 5 to 10 or from 6 to 10, including any intermediate values and subranges therebetween.

In some embodiments, the alkaline amino acid features pKa as described herein, and has a MW and hydrophilicity as described herein for a PAA.

Fatty acid salts according to some of the present embodiments can be represented by

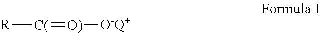

wherein R is a saturated or unsaturated, linear or branched, preferably unsubstituted alkylene chain of at least 4, preferably at least 6, carbon atoms in length, and Q⁺ is a cation, preferably a monovalent metal cation (e.g., Na+, K+), yet, any other cation, including non-metallic cations, are contemplated.

The fatty acid salt thus comprises an ionized form of a fatty acid (a carboxylate anion) and a cation.

The fatty acid from which the fatty acid salt is derived can be, without limitation, a saturated or unsaturated fatty acid having 4 or more carbon atoms, preferably 6 or more carbon atoms, for example, 8 or more carbon atoms, more or 10 or more carbon atoms, in length. Exemplary fatty acids include, such as, but not limited to, butyric acid, hexanoic acid, octanoic acid, decanoic acid, lauric acid, myristic acid, palmitic acid, stearic acid, oleic acid, arachidic acid, etc.

Preferably, the fatty acid is a medium chain fatty acid, having from 4 to 14, or from 4 to 12, or from 4 to 10, or from 6 to 16, or from 6 to 12, or from 6 to 10, or from 8 to 12, or from 8 to 10, carbon atoms in length. In some of these embodiments, the fatty acid is a saturated fatty acid. In some of these embodiments, the fatty acid is an unsubstituted fatty acid. In some of these embodiments, the fatty acid is unsubstituted saturated fatty acid.

Exemplary fatty acid salts include, but are not limited to, sodium butyrate, sodium hexanoate, sodium octanoate, sodium decanoate and sodium laurate.

Exemplary fatty acid salts include, but are not limited to, sodium octanoate and sodium decanoate.

In some of any of the embodiments described herein, a total concentration of the one or more small proton-accepting agent(s) (according to any of the respective embodiments described herein) in the composition is at least 1 weight percent of the total weight of the composition. In some such embodiments, the total concentration of the one or more small proton-accepting agent(s) is at least 2 weight percent. In some such embodiments, total concentration of the one or more small proton-accepting agent(s) is at least 3 weight percent. In some such embodiments, total concentration of the one or more small proton-accepting agent(s) is at least 4 weight percent. In some such embodiments, the total concentration of the one or more small proton-accepting agent(s) is at least 5 weight percent. In some such embodiments, the total concentration of the one or more small proton-accepting agent(s) is at least 10 weight percent. In some such embodiments, the total concentration of the one or more small proton-accepting agent(s) is at least 15 weight percent. In some embodiments, the total concentration of the one or more small proton-accepting agent(s) is at least 20 weight percent. In some embodiments, the concentration of the small proton-accepting agent is at least 25 weight percent. In some embodiments, total concentration of the one or more small proton-accepting agent(s) is at least 30 weight percent. In some embodiments, the total concentration of the one or more small proton-accepting agent(s) is at least 35 weight percent. In some embodiments, the total concentration of the one or more small proton-accepting agent(s) is at least 40 weight percent. In some embodiments, the total concentration of the one or more small proton-accepting agent(s) is at least 50 weight percent. In some embodiments, the total concentration of the one or more small proton-accepting agent(s) is at least 60 weight percent. In some embodiments, the total concentration of the one or more small proton-accepting agent(s) is at least 70 weight percent. In some embodiments, the total concentration of the one or more small proton-accepting agent(s) is at least 80 weight percent. In some embodiments, the total concentration of the one or more small proton-accepting agent(s) is at least 90 weight percent.

In some of any of the embodiments described herein, a total concentration of the one or more small proton-accepting agent(s) (according to any of the respective embodiments described herein) in the composition ranges from 1 to 90, or from 5 to 90 weight percent, of the total weight of the composition, or from 5 to 80, or from 5 to 70, or from 5 to 50, or from 5 to 40, or from 5 to 30, or from 10 to 90, or from 10 to 80, or from 10 to 70, or from 10 to 50, or from 10 to 40, or from 10 to 30, or from 20 to 90, or from 20 to 80, or from 20 to 70, or from 20 to 60, or from 20 to 50, or from 20 to 40, or from 20 to 30, or from 30 to 90, or from 30 to 80, or from 30 to 70, or from 30 to 60, or from 30 to 50, or from 30 to 40, or from 40 to 90, or from 40 to 80, or from 40 to 70, or from 40 to 60, or from 40 to 50, or from 50 to 90, or from 50 to 80, or from 50 to 70, or from 50 to 60, or from 60 to 90, or from 60 to 80, or from 60 to 70, or from 80 to 90, or from 70 to 80, or from 80 to 90, weight percent, of the total weight of the composition, including any intermediate values and subranges therebetween.

In some of any of the embodiments described herein, a total concentration of the one or more small proton-accepting agent(s) (according to any of the respective embodiments described herein) in the composition ranges from 1 to 50 weight percent, or from 5 to 50 weight percent, of the total weight of the composition, or from 5 to 40, or from 5 to 30, or from 5 to 20, or from 5 to 10, or from 10 to 50, or from 10 to 40, or from 10 to 30, or from 10 to 20, weight percent, of the total weight of the composition, including any intermediate values and subranges therebetween.

In some of any of the embodiments described herein, a total concentration of the one or more small proton-accepting agent(s) (according to any of the respective embodiments described herein) in the composition ranges from 10 to 60 weight percent, or from 10 to 50 weight percent, of the total weight of the composition, or from 10 to 40, or from 10 to 30, or from 20 to 60, or from 20 to 50, or from 20 to 40, or from 20 to 30, weight percent, of the total weight of the composition, including any intermediate values and subranges therebetween.

By “weight percent” (or w/w %, or % wt.) it is meant the weight of the indicated substance out of the total weight of a composition comprising same.

In some embodiments of any one of the embodiments described herein, a mol ratio of the one or more proton accepting agent(s) to the absorption enhancer in the composition is at least 1:20 (at least one proton accepting agent: absorption enhancer), optionally in a range of from 1:20 to 20:1, or from 1:10 to 10:1, or from or from 9:1 to 1:9, or from 1:8 to 8:1, or from 1:7 to 7:1, or from 1:6 to 6:1, or from 1:5 to 5:1, or from 1:4 to 4:1, or from 1:3 to 3:1, or from 1:2 to 2:1, including any intermediate values and subranges therebetween.

In some embodiments of any one of the embodiments described herein, a mol ratio of the one or more proton accepting agent(s) to the absorption enhancer is in a range of from 1:10 to 1:1, or from 1:5 to 1:1, or from 1:4 to 1:1, or from 1:3 to 1:1, or from 1:2 to 1:1, including any intermediate values and subranges therebetween, or is about 1:1.

In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a mol ratio of the one or more proton accepting agent(s) to the absorption enhancer is at least 0.05:1 (at least one proton accepting agent: absorption enhancer), optionally in a range of from 0.05:1 to 10:1, or from 0.05:1 to 9:1, or from 0.05:1 to 8:1, or from 0.05:1 to 7:1, or from 0.05:1 to 6:1, or from 0.05:1 to 5:1, or from 0.05:1 to 4:1, or from 0.05:1 to 3:1, or from 0.05:1 to 2:1, or from 0.05:1 to 1:1, or from 0.05:1 to 0.5:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a mol ratio of the one or more proton accepting agent(s) to the absorption enhancer is at least 0.06:1 (at least one proton accepting agent: absorption enhancer), optionally in a range of from 0.06:1 to 10:1, or from 0.06:1 to 9:1, or from 0.06:1 to 8:1, or from 0.06:1 to 7:1, or from 0.06:1 to 6:1, or from 0.06:1 to 5:1, or from 0.06:1 to 4:1, or from 0.06:1 to 3:1, or from 0.06:1 to 2:1, or from 0.06:1 to 1:1, or from 0.06:1 to 0.5:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a mol ratio of the one or more proton accepting agent(s) to the absorption enhancer is at least 0.07:1 (at least one proton accepting agent: absorption enhancer), optionally in a range of from 0.07:1 to 10:1, or from 0.07:1 to 9:1, or from 0.07:1 to 8:1, or from 0.07:1 to 7:1, or from 0.07:1 to 6:1, or from 0.07:1 to 5:1, or from 0.07:1 to 4:1, or from 0.07:1 to 3:1, or from 0.07:1 to 2:1, or from 0.07:1 to 1:1, or from 0.07:1 to 0.5:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a mol ratio of the one or more proton accepting agent(s) to the absorption enhancer is at least 0.08:1 (at least one proton accepting agent: absorption enhancer), optionally in a range of from 0.08:1 to 10:1, or from 0.08:1 to 9:1, or from 0.08:1 to 8:1, or from 0.08:1 to 7:1, or from 0.08:1 to 6:1, or from 0.08:1 to 5:1, or from 0.08:1 to 4:1, or from 0.08:1 to 3:1, or from 0.08:1 to 2:1, or from 0.08:1 to 1:1, or from 0.08:1 to 0.5:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a mol ratio of the one or more proton accepting agent(s) to the absorption enhancer is at least 0.09:1 (at least one proton accepting agent: absorption enhancer), optionally in a range of from 0.09:1 to 10:1, or from 0.09:1 to 9:1, or from 0.09:1 to 8:1, or from 0.09:1 to 7:1, or from 0.09:1 to 6:1, or from 0.09:1 to 5:1, or from 0.09:1 to 4:1, or from 0.09:1 to 3:1, or from 0.09:1 to 2:1, or from 0.09:1 to 1:1, or from 0.09:1 to 0.5:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a mol ratio of the one or more proton accepting agent(s) to the absorption enhancer is at least 0.1:1 (at least one proton accepting agent: absorption enhancer), optionally in a range of from 0.1:1 to 10:1, or from 0.1:1 to 9:1, or from 0.1:1 to 8:1, or from 0.1:1 to 7:1, or from 0.1:1 to 6:1, or from 0.1:1 to 5:1, or from 0.1:1 to 4:1, or from 0.1:1 to 3:1, or from 0.1:1 to 2:1, or from 0.1:1 to 1:1, or from 0.1:1 to 0.5:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the one or more proton accepting agent(s) to the absorption enhancer is at least 0.1:1 (at least one proton accepting agent: absorption enhancer), optionally in a range of from 0.1 to 1000:1, or from 0.1:1 to 500:1, or from 0.1:1 to 300:1, or from 0.1:1 to 200:1, or from 0.1:1 to 100:1, or from 0.1:1 to 50:1, or from 0.1:1 to 30:1, or from 0.1:1 to 20:1, or from 0.1:1 to 10:1, or from 0.1:1 to 5:1, or from 0.1:1 to 3:1, or from 0.1:1 to 2:1, or from 0.1 to 1:1 (at least one proton accepting agent: absorption enhancer), including any intermediate values and subranges therebetween. In some of the aforementioned embodiments, the absorption enhancer (8-N-(2-hydroxybenzoyl)aminocaprylic acid) or salt thereof a (e.g., sodium 8-N-(2-hydroxybenzoyl)aminocaprylate).

In some embodiments of any one of the embodiments described herein, a weight ratio of the one or more proton accepting agent(s) to the absorption enhancer is at least 0.2:1 (at least one proton accepting agent: absorption enhancer), optionally in a range of from 0.2 to 1000:1, or from 0.2:1 to 500:1, or from 0.2:1 to 300:1, or from 0.2:1 to 200:1, or from 0.2:1 to 100:1, or from 0.2:1 to 50:1, or from 0.2:1 to 30:1, or from 0.2:1 to 20:1, or from 0.2:1 to 10:1, or from 0.2:1 to 5:1, or from 0.2:1 to 3:1, or from 0.2:1 to 2:1, or from 0.2 to 1:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the one or more proton accepting agent(s) to the absorption enhancer is at least 0.3:1 (at least one proton accepting agent: absorption enhancer), optionally in a range of from 0.3 to 1000:1, or from 0.3:1 to 500:1, or from 0.3:1 to 300:1, or from 0.3:1 to 200:1, or from 0.3:1 to 100:1, or from 0.3:1 to 50:1, or from 0.3:1 to 30:1, or from 0.3:1 to 20:1, or from 0.3:1 to 10:1, or from 0.3:1 to 5:1, or from 0.3:1 to 3:1, or from 0.3:1 to 2:1, or from 0.3 to 1:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the one or more proton accepting agent(s) to the absorption enhancer is at least 0.4:1 (at least one proton accepting agent: absorption enhancer), optionally in a range of from 0.4 to 1000:1, or from 0.4:1 to 500:1, or from 0.4:1 to 300:1, or from 0.4:1 to 200:1, or from 0.4:1 to 100:1, or from 0.4:1 to 50:1, or from 0.4:1 to 30:1, or from 0.4:1 to 20:1, or from 0.4:1 to 10:1, or from 0.4:1 to 5:1, or from 0.4:1 to 3:1, or from 0.4:1 to 2:1, or from 0.4 to 1:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the one or more proton accepting agent(s) to the absorption enhancer is at least 0.5:1 (at least one proton accepting agent: absorption enhancer), optionally in a range of from 0.5 to 1000:1, or from 0.5:1 to 500:1, or from 0.5:1 to 300:1, or from 0.5:1 to 200:1, or from 0.5:1 to 100:1, or from 0.5:1 to 50:1, or from 0.5:1 to 30:1, or from 0.5:1 to 20:1, or from 0.5:1 to 10:1, or from 0.5:1 to 5:1, or from 0.5:1 to 3:1, or from 0.5:1 to 2:1, or from 0.5 to 1:1 (at least one proton accepting agent: absorption enhancer), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the one or more proton accepting agent(s) to the alkaline group-containing polymer is at least 0.1:1 (at least one proton accepting agent: alkaline group-containing polymer), optionally in a range of from 0.1:1 to 10:1, or from 0.1:1 to 9:1, or from 0.1:1 to 8:1, or from 0.1:1 to 7.1, or from 0.1:1 to 6.1, or from 0.1:1 to 5:1, or from 0.1:1 to 4:1, or from 0.1:1 to 3:1, or from 0.1:1 to 2:1, (at least one proton accepting agent: alkaline group-containing polymer), including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 0.3:1, optionally in a range of from 0.3:1 to 10:1, or from 0.3:1 to 9:1, or from 0.3:1 to 8:1, or from 0.3:1 to 7.1, or from 0.3:1 to 6.1, or from 0.3:1 to 5:1, or from 0.3:1 to 4:1, or from 0.3:1 to 3:1, or from 0.3:1 to 2:1, including any intermediate values and subranges therebetween. In some of the aforementioned embodiments, the alkaline-group containing polymer is sodium starch glycolate. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 0.5:1, optionally in a range of from 0.5:1 to 10:1, or from 0.5:1 to 9:1, or from 0.5:1 to 8:1, or from 0.5:1 to 7:1, or from 0.5:1 to 6:1, or from 0.5:1 to 5:1, or from 0.5:1 to 4:1, or from 0.5:1 to 3:1, or from 0.5:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 0.7:1, optionally in a range of from 0.7:1 to 10:1, or from 0.7:1 to 9:1, or from 0.7:1 to 8:1, or from 0.7:1 to 7:1, or from 0.7:1 to 6:1, or from 0.7:1 to 5:1, or from 0.7:1 to 4:1, or from 0.7:1 to 3:1, or from 0.7:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 0.9:1, optionally in a range of from 0.9:1 to 10:1, or from 0.9:1 to 9:1, or from 0.9:1 to 8:1, or from 0.9:1 to 7:1, or from 0.9:1 to 6:1, or from 0.9:1 to 5:1, or from 0.9:1 to 4:1, or from 0.9:1 to 3:1, or from 0.9:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 1.1:1, optionally in a range of from 1.1:1 to 10:1, or from 1.1:1 to 9:1, or from 1.1:1 to 8:1, or from 1.1:1 to 7:1, or from 1.1:1 to 6:1, or from 1.1:1 to 5:1, or from 1.1:1 to 4:1, or from 1.1:1 to 3:1, or from 1.1:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 1.3:1, optionally in a range of from 1.3:1 to 10:1, or from 1.3:1 to 9:1, or from 1.3:1 to 8:1, or from 1.3:1 to 7.1, or from 1.3:1 to 6.1, or from 1.3:1 to 5:1, or from 1.3:1 to 4:1, or from 1.3:1 to 3:1, or from 1.3:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least 1.5:1, optionally in a range of from 1.5:1 to 10:1, or from 1.5:1 to 9:1, or from 1.5:1 to 8:1, or from 1.5:1 to 7.1, or from 1.5:1 to 6.1, or from 1.5:1 to 5:1, or from 1.5:1 to 4:1, or from 1.5:1 to 3:1, or from 1.5:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least is at least 1.7:1, optionally in a range of from 1.7:1 to 10:1, or from 1.7:1 to 9:1, or from 1.7:1 to 8:1, or from 1.7:1 to 7.1, or from 1.7:1 to 6.1, or from 1.7:1 to 5:1, or from 1.7:1 to 4:1, or from 1.7:1 to 3:1, or from 1.7:1 to 2:1, including any intermediate values and subranges therebetween. In some of the aforementioned embodiments, the alkaline-group containing polymer is sodium starch glycolate.

In some embodiments of any one of the embodiments described herein, a weight ratio of the one or more proton accepting agent(s) to the alkaline group-containing polymer is at least is at least 1.9:1, optionally in a range of from 1.9:1 to 10:1, or from 1.9:1 to 9:1, or from 1.9:1 to 8:1, or from 1.9:1 to 7.1, or from 1.9:1 to 6.1, or from 1.9:1 to 5:1, or from 1.9:1 to 4:1, or from 1.9:1 to 3:1, or from 1.9:1 to 2:1, including any intermediate values and subranges therebetween. In some of the aforementioned embodiments, the alkaline-group containing polymer is sodium starch glycolate.

It is to be noted that when two of more proton accepting agents are used, and the combination provides at least an additive effect and optionally a synergistic effect, an amount of each PAA can be lower than its amount when used alone, as long as a total amount (concentration) is in accordance with any of the respective embodiments as described herein.

Whenever a weight ratio or a mol ratio are described herein, the ratio relates to one PAA, or, when two or more PAAs are included, the ratio relates to the total amount of the PAAs versus a salt of NAC or to each PAA alone versus a salt of NAC.

In certain embodiments, the amount or concentration of each of the absorption enhancer, the alkaline group-containing polymer, and the at least one proton-accepting agent within a composition can be determined by initially selecting an amount of the absorption enhancer based on the respective embodiments. Subsequently, a molar ratio between the absorption enhancer and the proton-accepting agent is selected. The remaining weight of the composition is then constituted by the alkaline group-containing polymer and the therapeutically active agent, and optionally additional agents (e.g., a lubricant). The total amount of each component also depends on a selected weight and/or size of the composition, in case of solid oral compositions.

In other embodiments, the amount or concentration of each of the absorption enhancer, the alkaline group-containing polymer, and the at least one proton-accepting agent within a composition may be ascertained by first selecting a combined total weight percent of the alkaline group-containing polymer and the proton-accepting agent. A molar ratio between the proton-accepting agent and the absorption enhancer is then determined to specify the quantity of the absorption enhancer. The residual weight of the composition is consequently allocated to the therapeutically active agent and optionally additional agents (e.g., a lubricant). The total amount of each component also depends on a selected weight and/or size of the composition, in case of solid oral compositions.

In other embodiments, the amount or concentration of the therapeutically active agent and the alkaline group-containing polymer is determined in accordance with any of the embodiments described herein. A molar ratio between the proton-accepting agent and the absorption enhancer is then determined and the remaining weight of the composition is divided accordingly between the absorption enhancer and the proton-accepting agent, and optionally additional agents (e.g., a lubricant). The total amount of each component also depends on a selected weight and/or size of the composition, in case of solid oral compositions.

In some embodiments of any one of the embodiments described herein relating to a small proton-accepting agent, a total amount of the small proton-accepting agent(s) in a composition as described herein, is at least 0.1 mg. In some embodiments, the amount of small proton-accepting agent(s) is at least 0.5 mg. In some embodiments, the amount of small proton-accepting agent(s) is at least 1 mg. In some embodiments, the amount of small proton-accepting agent(s) is at least 2 mg. In some embodiments, the amount of small proton-accepting agent(s) is at least 5 mg. In some embodiments, the amount of small proton-accepting agent(s) is at least 10 mg. In some embodiments, the amount of small proton-accepting agent(s) is at least 20 mg.

In some embodiments of any one of the embodiments described herein relating to a small proton-accepting agent, a total amount of the small proton-accepting agent(s) in a composition as described herein, is in a range of from 10 to 60, or from 10 to 50, or from 20 to 60, or from 20 to 50, or from 30 to 60, or from 30 to 50 mg, including any intermediate values and subranges therebetween. In some embodiments of any one of the embodiments described herein relating to a small proton-accepting agent, a total amount of the small proton-accepting agent(s) in a composition as described herein, is in a range of from 0.1 to 50, or from 0.1 to 40, or from 0.1 to 30, or from 0.1 to 20, or from 1 to 50, or from 1 to 40, or from 1 to 30, or from 1 to 20, or from 1 to 10, mg, including any intermediate values and subranges therebetween.

In some embodiments of any one of the embodiments described herein relating to a small proton-accepting agent, a total amount of the small proton-accepting agent(s) in a composition as described herein, is in a range of from 0.1 to 50, or from 0.1 to 40, or from 0.1 to 30, or from 0.1 to 20, or from 1 to 50, or from 1 to 40, or from 1 to 30, or from 1 to 20, or from 1 to 10, mg, including any intermediate values and subranges therebetween.

In some embodiments of any one of the embodiments described herein relating to a small proton-accepting agent, a total amount of the small proton-accepting agent(s) in a composition as described herein, is in a range of from 50 to 500, or from 50 to 400, or from 50 to 300, or from 50 to 200, or from 50 to 100, or from 100 to 200, or from 100 to 300, or from 100 to 400, or from 100 to 500, or from 200 to 500, or from 200 to 400, or from 200 to 300, or from 300 to 500, or from 300 to 400, or from 400 to 500 mg, including any intermediate values and subranges therebetween.

In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

The effect of the small proton-accepting agent(s) on the percentage recovery of SNAC may be determined by comparing percentage recovery of a composition according to embodiments of the invention with a corresponding composition comprising the same amount of other ingredients other than the small proton-accepting agent, such that the total mass is lower (due to the absence of small proton-accepting agent). In such embodiments, both compositions are in a form of a tablet, and the tablets have the same cross-section (e.g., diameter of a circular cross-section), and the tablet with a lower weight is thinner in the axis perpendicular to the cross-section of the tablet.

In some embodiments of any one of the embodiments described herein relating to a small proton-accepting agent, the small proton-accepting agent(s) is or comprises an antacid as described herein in any of the respective embodiments, for example, sodium carbonate, and a total amount the small proton-accepting agent(s) in a composition as described herein in any of the respective embodiment ranges from 10 to 50, or from 10 to 40, or from 10 to 30, or from 20 to 50, or from 20 to 40, or from 20 to 30, mg, including any intermediate values and subranges tehrebetween, although higher amounts are also contemplated as described herein in respective embodiments. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein relating to a small proton-accepting agent, the small proton-accepting agent(s) is or comprises an antacid as described herein in any of the respective embodiments, for example, sodium carbonate, and a total amount the small proton-accepting agent(s) in a composition as described herein in any of the respective embodiment ranges from 10 to 50, or from 10 to 40, or from 10 to 30, weight percent, of the total weight of the composition, including any intermediate values and subranges therebetween, although higher amounts are also contemplated as described herein in respective embodiments. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein relating to a small proton-accepting agent, the small proton-accepting agent(s) is or comprises an alkaline amino acid as described herein in any of the respective embodiments, for example, arginine, and a total amount the small proton-accepting agent(s) in a composition as described herein in any of the respective embodiment ranges from 10 to 80, or from 10 to 70, or from 10 to 60, or from 20 to 80, or from 20 to 70, or from 20 to 60, or from 30 to 90, or from 30 to 70, or from 30 to 60, or from 30 to 50, or from 40 to 80, or from 40 to 70, or from 40 to 60, mg, including any intermediate values and subranges tehrebetween, although higher amounts are also contemplated as described herein in respective embodiments. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein relating to a small proton-accepting agent, the small proton-accepting agent(s) is or comprises an alkaline amino acid as described herein in any of the respective embodiments, for example, arginine, and a total amount the small proton-accepting agent(s) in a composition as described herein in any of the respective embodiment ranges from 10 to 50, or from 10 to 40, or from 20 to 40, weight percent, of the total weight of the composition, including any intermediate values and subranges therebetween, although higher amounts are also contemplated as described herein in respective embodiments. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein relating to a small proton-accepting agent, the small proton-accepting agent(s) is or comprises a fatty acid salt as described herein in any of the respective embodiments, for example, sodium decanoate, and a total amount the small proton-accepting agent(s) in a composition as described herein in any of the respective embodiment ranges from 10 to 80, or from 10 to 70, or from 10 to 60, or from 20 to 80, or from 20 to 70, or from 20 to 60, or from 30 to 90, or from 30 to 70, or from 30 to 60, or from 30 to 50, or from 40 to 80, or from 40 to 70, or from 40 to 60, mg, including any intermediate values and subranges tehrebetween, although higher amounts are also contemplated as described herein in respective embodiments. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein relating to a small proton-accepting agent, the small proton-accepting agent(s) is or comprises an fatty acid salt as described herein in any of the respective embodiments, for example, sodium decanoate, and a total amount the small proton-accepting agent(s) in a composition as described herein in any of the respective embodiment ranges from 10 to 50, or from 10 to 40, or from 20 to 40, weight percent, of the total weight of the composition, although higher amounts are also contemplated as described herein in respective embodiments. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the aforementioned embodiments, the alkaline polymer is or comprises a polysaccharide, and in some embodiments it is or comprises sodium starch glycolate (SGG).

The effect of the small proton-accepting agent(s) on absorption of the therapeutically active agent may optionally be determined by comparing absorption upon oral administration of a composition according to embodiments of the invention with a corresponding composition comprising the same amount of other ingredients other than the small proton-accepting agent, such that the total mass is lower (due to the absence of small proton-accepting agent). In such embodiments, both compositions are in a form of a tablet, and the tablets have the same cross-section (e.g., diameter of a circular cross-section), and the tablet with a lower weight is thinner in the axis perpendicular to the cross-section of the tablet.

In some embodiments of any one of the embodiments described herein, a Cmax and/or bioavailability of the composition (i.e., of the therapeutically active agent in the composition) upon oral administration is at least 20% higher than (120% of the level of) a Cmax and/or bioavailability of a corresponding composition without the small proton-accepting agent. In some embodiments, the Cmax and/or bioavailability is at least 50% higher than (150% of the level of) the Cmax and/or bioavailability upon oral administration of a corresponding composition without small proton-accepting agent. In some embodiments, the Cmax and/or bioavailability is at least twice (200% of the level of) the Cmax and/or bioavailability upon oral administration of a corresponding composition without the small proton-accepting agent. In some embodiments, the Cmax and/or bioavailability is at least four-fold (400% of the level of) the Cmax and/or bioavailability upon oral administration of a corresponding composition without the small proton-accepting agent. In some embodiments, the Cmax and/or bioavailability is at least six-fold (600% of the level of) the Cmax and/or bioavailability upon oral administration of a corresponding composition without the small proton-accepting agent. In some embodiments, the Cmax and/or bioavailability is at least ten-fold (1000% of the level of) the Cmax and/or bioavailability upon oral administration of a corresponding composition without the small proton-accepting agent.

Cmax and/or bioavailability may optionally be determined by administering the composition orally to subjects (e.g., human subjects) and determining a level of the therapeutically active agent in the blood at frequent intervals by taking blood samples.

Bioavailability may be determined by comparing a ratio of an area under the curve upon oral administration (e.g., over the course of 12 or 24 hours) to an area under curve over the same time period for intravenously administered therapeutically active agent, using standard techniques (e.g., data processing algorithms) known in the art. The therapeutically active agent may be injected at a lower dose (e.g., for safety reasons) and the areas under curve normalized to the total amount administered.

### Absorption Enhancer:

According to preferred embodiments of any of the embodiments described herein, the composition according to any of the respective embodiments described herein comprises an effective amount of an absorption enhancer, i.e., an amount of absorption enhancer effective for enhancing absorption of the therapeutically active agent in the composition.

Herein, the term “absorption enhancer” refers to a compound known to enhance absorption of macromolecular drugs (e.g., compounds having a molecular weight of at least 1 kDa) and/or BCS Class III drugs (e.g., as described herein) from the gastrointestinal tract into the circulation upon oral administration of the drug. The person skilled in the art will be aware of many such absorption enhancers. In some of any of the respective embodiments, the absorption enhancer is a compound which enhances absorption of macromolecular drugs.

In some of any of the embodiments described herein, the absorption enhancer is a fatty acid, preferably with a terminal N-(2-hydroxybenzoyl)amino group (at the omega position, i.e., the terminus distal from the carboxylate group of the fatty acid), or a salt thereof (e.g., a monosodium or disodium salt).

The substituted fatty acid is preferably from 4 to 20 carbon atoms in length, optionally from 4 to 18 carbon atoms in length, optionally from 4 to 16 carbon atoms in length, optionally from 4 to 14 carbon atoms in length, optionally from 4 to 12 carbon atoms in length and optionally from 4 to 10 carbon atoms in length, including any intermediate values and subranges therebetween. In some of any of the embodiments described herein, the fatty acid is from 6 to 20 carbon atoms in length, optionally from 6 to 18 carbon atoms in length, optionally from 6 to 16 carbon atoms in length, optionally from 6 to 14 carbon atoms in length, optionally from 6 to 12 carbon atoms in length, optionally from 6 to 10 carbon atoms in length, and optionally from 8 to 10 carbon atoms in length, including any intermediate values and subranges therebetween. The fatty acid moiety may be saturated (e.g., as are caprylic acid in 8-N-(2-hydroxybenzoyl)aminocaprylic acid and decanoic acid in 10-N-(2-hydroxybenzoyl)aminodecanoic acid) or unsaturated (i.e., comprising at least one unsaturated carbon-carbon bond).

Examples of suitable fatty acids for substituted (e.g., by a terminal N-(2-hydroxybenzoyl)amino group) include, without limitation, butanoic acid, caprylic acid and decanoic acid.

The N-(2-hydroxybenzoyl)amino group may optionally be substituted or non-substituted (e.g., on the aromatic ring thereof). Suitable substituents include, for example, halo (optionally chloro) and alkoxy (optionally methoxy). Examples of substituted N-(2-hydroxybenzoyl)amino groups include, without limitation, N-(5-chlorosalicyloyl)amino, N-(4-chloro-2-hydroxybenzoyl)amino, and N-(2-hydroxy-4-methoxybenzoyl)amino.

Examples of suitable absorption enhancers include, without limitation, NAC (8-N-(2-hydroxybenzoyl)aminocaprylic acid) and NAD (10-N-(2-hydroxybenzoyl)aminodecanoic acid) and salts thereof (e.g., monosodium and disodium salts); as well as derivatives thereof (e.g., derivatives substituted by chloro and/or methoxy) such as 5-CNAC (8-N-(5-chlorosalicyloyl)aminocaprylic acid) and 4-MOAC (8-N-(2-hydroxy-4-methoxybenzoyl)aminocaprylic acid) and salts thereof (e.g., monosodium and disodium salts). 4-CNAB (4-N-(2-hydroxy-4-chlorobenzoyl)aminobutanoic acid) and salts thereof (e.g., monosodium and disodium salts) are additional examples of a suitable absorption enhancer.

In some of any of the embodiments described herein, the absorption enhancer is in a form of a salt thereof, for example, a sodium salt. In exemplary embodiments, the sodium salt is a monosodium salt.

In some of any of the embodiments described herein, the absorption enhancer is NAC or NAD, or a salt thereof. In some such embodiments, the absorption enhancer is NAC or a salt thereof.

As shown below, the structure of NAD (depicted as a sodium salt thereof, also referred to as “SNAD”) differs from that of NAC (depicted as a sodium salt thereof, also referred to as “SNAC”) only in the length of the fatty acid moiety. Additional absorption enhancers related to NAC and NAD, based on different fatty acid lengths, will be readily apparent to the skilled person.

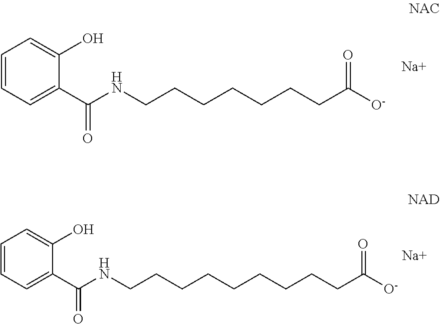 [SMILES: O=C([O-])CCCCCCCNC(=O)c1ccccc1O ; [Na+] ; O=C([O-])CCCCCCCCCNC(=O)c1ccccc1O ; [Na+]]

Without being bound by any particular theory, it is believed that many absorption enhancers tend to be more active (at enhancing absorption) in an ionic form (e.g., a carboxylate anion, such as a fatty acid anion) than in a less soluble non-ionic form (e.g., a carboxylic acid, such as a fatty acid), and that overall activity of the absorption enhancer is strongly dependent on concentration of the more active form. For example, the activity of the less soluble non-ionic form may be reduced by precipitation. It is further believed that the amount of absorption enhancer in ionic form upon dissolution following oral administration may be enhanced by providing the absorption enhancer in a form of a salt and/or by controlling a local pH in the vicinity of the absorption enhancer upon oral administration (e.g., by acid neutralization by alkaline groups of a polymer described herein and/or even by other molecules of absorption enhancer).

According to some of any of the embodiments described herein, the absorption enhancer is an ionizable material (compound), and in some of these embodiments, the absorption enhancer is more active in its ionic form.

According to some of any of the embodiments described herein, the absorption enhancer is inactivated by stomach acid, as described herein, and in some of these embodiments, the absorption enhancer is ionizable, is more active in its ionic form, and at least a portion of the absorption enhancer is in a non-ionic (less active) form when contacted with stomach acid (due, e.g., to precipitation).

It is further believed that formation of a viscous medium may enhance the effect of absorption enhancers which are not inactivated by stomach acid, e.g., by maintaining a higher local concentration of absorption enhancer in the vicinity of the therapeutically active agent and/or facilitating adhesion of the composition to the stomach wall (e.g., as described herein).

Additionally or alternatively, the control over local pH associated with the polymer may protect the therapeutically active agent from inactivation by stomach acid (regardless of whether the absorption enhancer is inactivated by stomach acid), for example, wherein the agent is a polypeptide and inactivation is associated with pepsin activity in the presence of a suitably acidic pH (e.g., as discussed elsewhere herein).

According to some of any of the embodiments described herein, the absorption enhancer is not inactivated by stomach acid, for example, it is not ionizable and/or its ionic form and its non-ionic forms exhibit the same activity and/or it is not converted to a less active form when contacted with stomach acid (for example, the absorption enhancer is acidic by itself).

Examples of absorption enhancers which are not expected to be inactivated by stomach acid include, without limitation, alkyl fatty acid esters (e.g., isopropyl myristate); phospholipids (e.g., phosphatidyl choline); quaternary ammonium salts, such as tetraalkyl ammonium salts (e.g., cetyltrimethylammonium salts, such as cetyltrimethylammonium bromide) and alkyl pyridinium salts (e.g., cetylpyridinium salts such as cetylpyridinium chloride); nonionic surfactants such as sorbitan-fatty acid esters (e.g., sorbitan monolaurate, sorbitan monostearate, sorbitan tristearate) and polysorbates (i.e., ethoxylated sorbitan-fatty acid esters, e.g., polysorbate 20, polysorbate 40, polysorbate 60 and polysorbate 80); and N-dodecylcaprolactam.

In some of any one of the embodiments described herein, a concentration of the absorption enhancer (according to any of the respective embodiments described herein) in a composition (according to any of the respective embodiments described herein) is at least 10 weight percent; for example, from 10 to 90 weight percent, or from 10 to 80 weight percent, or from 10 to 70 weight percent, or from 10 to 60 weight percent, or from 10 to 50 weight percent, or from 10 to 40 weight percent, or from 10 to 30 weight percent, including any intermediate values and subranges therebetween. In some such embodiments, the concentration of the absorption enhancer is at least 20 weight percent; for example, from 20 to 90 weight percent, or from 20 to 80 weight percent, or from 20 to 70 weight percent, or from 20 to 60 weight percent, or from 20 to 50 weight percent, or from 20 to 40 weight percent, including any intermediate values and subranges therebetween. In some embodiments, the concentration of the absorption enhancer is at least 30 weight percent; for example, from 30 to 90 weight percent, or from 30 to 80 weight percent, or from 30 to 70 weight percent, or from 30 to 60 weight percent, or from 30 to 50 weight percent, including any intermediate values and subranges therebetween. In some embodiments, the concentration of the absorption enhancer is at least 40 weight percent; for example, from 40 to 90 weight percent, or from 40 to 80 weight percent, or from 40 to 70 weight percent, or from 40 to 60 weight percent, including any intermediate values and subranges therebetween. In some embodiments, the concentration of the absorption enhancer is at least 50 weight percent; for example, from 50 to 90 weight percent, or from 50 to 80 weight percent, or from 50 to 70 weight percent, including any intermediate values and subranges therebetween. In some embodiments, the concentration of the absorption enhancer is at least 60 weight percent; for example, from 60 to 90 weight percent, or from 60 to 80 weight percent, including any intermediate values and subranges therebetween. In some embodiments, the concentration of the absorption enhancer is at least 70 weight percent; for example, from 70 to 90 weight percent or from 70 to 80 weight percent, including any intermediate values and subranges therebetween. In some embodiments, the concentration of the absorption enhancer is at least 80 weight percent; for example, from 80 to 90 weight percent, including any intermediate values and subranges therebetween.

In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the polymer comprising the alkaline groups (according to any of the respective embodiments described herein) and the proton accepting agent (according to any of the respective embodiments described herein) is at least 80 weight percent (e.g., from 80 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent (e.g., according to any of the respective embodiments described herein). Alternatively or additionally, in some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the proton accepting agent (according to any of the respective embodiments described herein), and the polymer comprising the alkaline groups (according to any of the respective embodiments described herein) is at least 90 weight percent (e.g., from 90 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). Alternatively or additionally, in some embodiments, the concentration of the polymer comprising the alkaline groups is at least 5 weight percent or at least weight percent or at least 20 weight percent or at least 30 weight percent or at least 40 weight percent or at least 50 weight percent or at least 60 weight percent or at least 70 weight percent or 10 at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the proton accepting agent (according to any of the respective embodiments described herein), and the polymer comprising alkaline groups (according to any of the respective embodiments described herein) is at least 95 weight percent (e.g., from 95 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). Alternatively or additionally, in some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the proton accepting agent (according to any of the respective embodiments described herein), and the polymer comprising alkaline groups (according to any of the respective embodiments described herein) is at least 98 weight percent. In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the proton accepting agent (according to any of the respective embodiments described herein), and the polymer comprising the alkaline groups (according to any of the respective embodiments described herein) is at least 80 weight percent (e.g., from 80 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent (e.g., according to any of the respective embodiments described herein). Alternatively or additionally, in some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the proton accepting agent (according to any of the respective embodiments described herein), and the polymer comprising the alkaline groups (according to any of the respective embodiments described herein) is at least 90 weight percent (e.g., from 90 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). Alternatively or additionally, in some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the polymer comprising alkaline groups (according to any of the respective embodiments described herein) and the proton accepting agent (according to any of the respective embodiments described herein) is at least 95 weight percent (e.g., from 95 to 100 weight percent, including any intermediate values and subranges therebetween). In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). Alternatively or additionally, in some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), the polymer comprising alkaline groups (according to any of the respective embodiments described herein) and the proton accepting agent (according to any of the respective embodiments described herein) is at least 98 weight percent. In some such embodiments, the concentration of the absorption enhancer is at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent or at least 80 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any of the aforementioned embodiments, the alkaline polymer is or comprises a polysaccharide, and in some embodiments it is or comprises sodium starch glycolate (SGG).

In some of any one of the embodiments relating to a unit dosage form described herein, an amount of the absorption enhancer in the unit dosage form is at least 25 mg; for example, from 25 to 1000 mg or from 25 to 500 mg or from 25 to 250 mg or from 25 to 100 mg or from 25 to 50 mg, including any intermediate values and subranges therebetween. In some embodiments, the amount of the absorption enhancer in the unit dosage form is at least 50 mg; for example, from 50 to 1000 mg or from 50 to 500 mg or from 50 to 250 mg or from 50 to 100 mg, including any intermediate values and subranges therebetween. In some embodiments, the amount of the absorption enhancer in the unit dosage form is at least 75 mg; for example, from 75 to 1000 mg or from 75 to 500 mg or from 75 to 250 mg, including any intermediate values and subranges therebetween. In some embodiments, the amount of the absorption enhancer in the unit dosage form is at least 100 mg; for example, from 100 to 1000 mg or from 100 to 500 mg or from 100 to 250 mg, including any intermediate values and subranges therebetween. In some embodiments, the amount of the absorption enhancer in the unit dosage form is at least 150 mg; for example, from 150 to 1000 mg or from 150 to 500 mg or from 150 to 250 mg, including any intermediate values and subranges therebetween. In some embodiments, the amount of the absorption enhancer in the unit dosage form is at least 200 mg; for example, from 200 to 1000 mg or from 200 to 500 mg, including any intermediate values and subranges therebetween. In some embodiments, the amount of the absorption enhancer in the unit dosage form is at least 300 mg; for example, from 300 to 1000 mg or from 300 to 500 mg, including any intermediate values and subranges therebetween. In some of any of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the aforementioned embodiments, the alkaline polymer is or comprises a polysachharide, and in some embodiments it is or comprises sodium starch glycolate (SGG).

In some of any one of the embodiments described herein, a concentration of the absorption enhancer (according to any of the respective embodiments described herein) in a composition (according to any of the respective embodiments described herein) is lower by at least 10% compared to a composition without a sPAA as described herein.

In some of any one of the embodiments described herein, a concentration of the absorption enhancer (according to any of the respective embodiments described herein) in a composition (according to any of the respective embodiments described herein) is no more than 90 weight percent; or no more than 80 weight percent, or no more than 70 weight percent.

In some of any one of the embodiments described herein, a concentration of the absorption enhancer (according to any of the respective embodiments described herein) in a composition (according to any of the respective embodiments described herein) is in a range of from 10 to 80 weight percent, or from 10 to 70 weight percent, or from 10 to 60 weight percent, or from 10 to 50 weight percent, or from 10 to 30 weight percent, including any intermediate values and subranges therebetween. In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), and the proton accepting agent (according to any of the respective embodiments described herein) is at least 50 weight percent (e.g., from 50 to 95, or from 50 to 90, or from 50 to 85, or from 50 to 80, or from 50 to 75, or from 50 to 70, or from 50 to 60, weight percent, including any intermediate values and subranges therebetween). In some such embodiments, the concentration of the polymer comprising the alkaline groups is at least 5 weight percent or at least 10 weight percent or at least 20 weight percent or at least 30 weight percent or at least 40 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), and the proton accepting agent (according to any of the respective embodiments described herein), is at least 40 weight percent (e.g., from 40 to 95, or from 40 to 90, or from 40 to 84, or from 40 to 80, or from 40 to 75, or from 40 to 70, or from 40 to 60, or from 40 to 50, weight percent, including any intermediate values and subranges therebetween). In some such embodiments, the concentration of the polymer comprising the alkaline groups is at least 5 weight percent or at least 10 weight percent or at least 20 weight percent or at least 30 weight percent or at least 40 weight percent or at least 50 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), and the proton accepting agent (according to any of the respective embodiments described herein), is at least 30 weight percent (e.g., from 30 to 95, or from 30 to 90, or from 30 to 85, or from 30 to 80, or from 30 to 75, or from 30 to 70, or from 30 to 65, or from 30 to 60, or from 30 to 55, or from 30 to 50, or from 30 to 40 weight percent, including any intermediate values and subranges therebetween). In some such embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments described herein, a total concentration of the absorption enhancer (according to any of the respective embodiments described herein), and the proton accepting agent (according to any of the respective embodiments described herein), and the polymer comprising alkaline groups (according to any of the respective embodiments described herein) is at least 20 weight percent (e.g., from 20 to 95, or from 20 to 90, or from 20 to 85, or from 20 to 80, or from 20 to 75, or from 20 to 70, or from 20 to 65, or from 20 to 60, or from 20 to 55, or from 20 to 50, or from 20 to 40, or from 20 to 30, weight percent, including any intermediate values and subranges therebetween). In some such embodiments, the concentration of the polymer comprising the alkaline groups is at least 5 weight percent or at least 10 weight percent or at least 20 weight percent or at least 30 weight percent or at least 40 weight percent or at least 50 weight percent or at least 60 weight percent or at least 70 weight percent (e.g., according to any of the respective embodiments described herein). In some of any of the aforementioned embodiments, the absorption enhancer is NAC, NAD, 5-CNAC, 4-MOAC and/or 4-CNAB, or a salt thereof (e.g., a sodium salt thereof).

In some of any one of the embodiments relating to a unit dosage form described herein, a total amount of the absorption enhancer and the sPAA in the unit dosage form is at least 25 mg; for example, from 25 to 1000 mg or from 25 to 500 mg or from 25 to 250 mg or from 25 to 100 mg or from 25 to 50 mg, including any intermediate values and subranges therebetween. In some embodiments, a total amount of the absorption enhancer and the sPAA in the unit dosage form is at least 50 mg; for example, from 50 to 1000 mg or from 50 to 500 mg or from 50 to 250 mg or from 50 to 100 mg, including any intermediate values and subranges therebetween. In some embodiments, a total amount of the absorption enhancer and the sPAA in the unit dosage form is at least 75 mg; for example, from 75 to 1000 mg or from 75 to 500 mg or from 75 to 250 mg, including any intermediate values and subranges therebetween. In some embodiments, a total amount of the absorption enhancer and the sPAA in the unit dosage form is at least 100 mg; for example, from 100 to 1000 mg or from 100 to 500 mg or from 100 to 250 mg, including any intermediate values and subranges therebetween. In some embodiments, a total amount of the absorption enhancer and the sPAA in the unit dosage form is at least 150 mg; for example, from 150 to 1000 mg or from 150 to 500 mg or from 150 to 250 mg, including any intermediate values and subranges therebetween. In some embodiments, a total amount of the absorption enhancer and the sPAA in the unit dosage form is at least 200 mg; for example, from 200 to 1000 mg or from 200 to 500 mg, including any intermediate values and subranges therebetween. In some embodiments, a total amount of the absorption enhancer and the sPAA in the unit dosage form is at least 300 mg; for example, from 300 to 1000 mg or from 300 to 500 mg, including any intermediate values and subranges therebetween. In some of any of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the aforementioned embodiments, the alkaline polymer is or comprises a polysachharide, and in some embodiments it is or comprises sodium starch glycolate (SGG).

In some embodiments of any one of the embodiments described herein, a weight ratio of the absorption enhancer to the therapeutically active agent in the composition is at least 1:1 (absorption enhancer: therapeutically active agent), optionally in a range of from 1:1 to 1000:1, or from 1:1 to 500:1, or from 1:1 to 300:1, or from 1:1 to 200:1, or from 1:1 to 100:1, or from 1:1 to 50:1, or from 1:1 to 30:1, or from 1:1 to 20:1, or from 1:1 to 10:1, or from 1:1 to 5:1, or from 1:1 to 3:1, or from 1:1 to 2:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 2:1 (absorption enhancer: therapeutically active agent), optionally in a range of from 2:1 to 1000:1, or from 2:1 to 500:1, or from 2:1 to 300:1, or from 2:1 to 200:1, or from 2:1 to 100:1, or from 2:1 to 50:1, or from 2:1 to 30:1, or from 2:1 to 20:1, or from 2:1 to 10:1, or from 2:1 to 5:1, or from 2:1 to 3:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 3:1 (absorption enhancer: therapeutically active agent), optionally in a range of from 3:1 to 1000:1, or from 3:1 to 500:1, or from 3:1 to 300:1, or from 3:1 to 200:1, or from 3:1 to 100:1, or from 3:1 to 50:1, or from 3:1 to 30:1, or from 3:1 to 20:1, or from 3:1 to 10:1, or from 3:1 to 5:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to the therapeutically active agent in the composition is at least 5:1 (absorption enhancer: therapeutically active agent), optionally in a range of from 5:1 to 1000:1, or from 5:1 to 500:1, or from 5:1 to 300:1, or from 5:1 to 200:1, or from 5:1 to 100:1, or from 5:1 to 50:1, or from 5:1 to 30:1, or from 5:1 to 20:1, or from 5:1 to 10:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 10:1 (absorption enhancer: therapeutically active agent), optionally in a range of from 10:1 to 1000:1, or from 10:1 to 500:1, or from 10:1 to 300:1, or from 10:1 to 200:1, or from 10:1 to 100:1, or from 10:1 to 50:1, or from 10:1 to 30:1, or from 10:1 to 20:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 20:1 (absorption enhancer: therapeutically active agent), optionally in a range of from 20:1 to 1000:1, or from 20:1 to 500:1, or from 20:1 to 300:1, or from 20:1 to 200:1, or from 20:1 to 100:1, or from 20:1 to 50:1, or from 20:1 to 30:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 30:1 (absorption enhancer: therapeutically active agent), optionally in a range of from 30:1 to 1000:1, or from 30:1 to 500:1, or from 30:1 to 300:1, or from 30:1 to 200:1, or from 30:1 to 100:1, or from 30:1 to 50:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 50:1 (absorption enhancer: therapeutically active agent), optionally in a range of from 50:1 to 1000:1, or from 50:1 to 500:1, or from 50:1 to 300:1, or from 50:1 to 200:1, or from 50:1 to 100:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 100:1 (absorption enhancer: therapeutically active agent), optionally in a range of from 100:1 to 1000:1, or from 100:1 to 500:1, or from 100:1 to 300:1, or from 100:1 to 200:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 200:1 (absorption enhancer: therapeutically active agent), optionally in a range of from 200:1 to 1000:1, or from 200:1 to 500:1, or from 200:1 to 300:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 300:1 (absorption enhancer: therapeutically active agent), optionally in a range of from 300:1 to 1000:1, or from 300:1 to 500:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some embodiments of any one of the embodiments described herein, a weight ratio of the absorption enhancer to therapeutically active agent in the composition is at least 500:1, optionally in a range of from 500:1 to 1000:1 (absorption enhancer: therapeutically active agent), including any intermediate values and subranges therebetween. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the aforementioned embodiments, the alkaline polymer is or comprises a polysachharide, and in some embodiments it is or comprises sodium starch glycolate (SGG).

### Therapeutically Active Agent:

Compositions described herein are particularly suitable for enhancing the absorption of therapeutically active agents whose absorption upon oral administration is limited, for example, by a large molecular weight, strong hydrophilicity (e.g., which inhibits crossing of lipid membranes in the gastrointestinal tract), and/or degradation in the gastrointestinal tract (e.g., by proteolysis), for example, degradation in the stomach by pepsin and/or stomach acid.

In some embodiments of any one of the embodiments described herein, the therapeutically active agent according to any of the respective embodiments described herein has a molecular weight of at least 0.5 kDa. In some embodiments, the molecular weight is in a range of from 0.5 to 150 kDa, including any intermediate values and subranges therebetween. In some embodiments, the molecular weight is in a range of from 0.5 to 100 kDa, including any intermediate values and subranges therebetween. In some embodiments, the molecular weight is in a range of from 0.5 to 75 kDa, including any intermediate values and subranges therebetween. In some embodiments, the molecular weight is in a range of from 0.5 to 50 kDa, including any intermediate values and subranges therebetween. In some embodiments, the molecular weight is in a range of from 0.5 to 30 kDa, including any intermediate values and subranges therebetween. In some embodiments, the molecular weight is in a range of from 0.5 to 20 kDa, including any intermediate values and subranges therebetween. In some embodiments, the molecular weight is in a range of from 0.5 to 10 kDa, including any intermediate values and subranges therebetween. In some embodiments, the molecular weight is in a range of from 0.5 to 7.5 kDa, including any intermediate values and subranges therebetween. In some embodiments, the molecular weight is in a range of from 0.5 to 5 kDa, including any intermediate values and subranges therebetween.

In some embodiments of any one of the embodiments described herein, the therapeutically active agent has a molecular weight of at least 1 kDa. In some embodiments, the molecular weight is in a range of from 1 to 150 kDa, including any intermediate values and subranges therebetween. In some embodiments, the molecular weight is in a range of from 1 to 100 kDa, including any intermediate values and subranges therebetween. In some embodiments, the molecular weight is in a range of from 1 to 75 kDa, including any intermediate values and subranges therebetween. In some embodiments, the molecular weight is in a range of from 1 to 50 kDa, including any intermediate values and subranges therebetween. In some embodiments, the molecular weight is in a range of from 1 to 30 kDa, including any intermediate values and subranges therebetween. In some embodiments, the molecular weight is in a range of from 1 to 20 kDa, including any intermediate values and subranges therebetween. In some embodiments, the molecular weight is in a range of from 1 to 10 kDa, including any intermediate values and subranges therebetween. In some embodiments, the molecular weight is in a range of from 1 to 7.5 kDa, including any intermediate values and subranges therebetween. In some embodiments, the molecular weight is in a range of from 1 to 5 kDa, including any intermediate values and subranges therebetween.

In some embodiments of any one of the embodiments described herein, the therapeutically active agent has a molecular weight of at least 2 kDa. In some embodiments, the molecular weight is in a range of from 2 to 150 kDa, including any intermediate values and subranges therebetween. In some embodiments, the molecular weight is in a range of from 2 to 100 kDa, including any intermediate values and subranges therebetween. In some embodiments, the molecular weight is in a range of from 2 to 75 kDa, including any intermediate values and subranges therebetween. In some embodiments, the molecular weight is in a range of from 2 to 50 kDa, including any intermediate values and subranges therebetween. In some embodiments, the molecular weight is in a range of from 2 to 30 kDa, including any intermediate values and subranges therebetween. In some embodiments, the molecular weight is in a range of from 2 to 20 kDa, including any intermediate values and subranges therebetween. In some embodiments, the molecular weight is in a range of from 2 to 10 kDa, including any intermediate values and subranges therebetween. In some embodiments, the molecular weight is in a range of from 2 to 7.5 kDa, including any intermediate values and subranges therebetween. In some embodiments, the molecular weight is in a range of from 2 to 5 kDa, including any intermediate values and subranges therebetween.

In some embodiments of any one of the embodiments described herein, the therapeutically active agent has a molecular weight of at least 3 kDa. In some embodiments, the molecular weight is in a range of from 3 to 150 kDa, including any intermediate values and subranges therebetween. In some embodiments, the molecular weight is in a range of from 3 to 100 kDa, including any intermediate values and subranges therebetween. In some embodiments, the molecular weight is in a range of from 3 to 75 kDa, including any intermediate values and subranges therebetween. In some embodiments, the molecular weight is in a range of from 3 to 50 kDa, including any intermediate values and subranges therebetween. In some embodiments, the molecular weight is in a range of from 3 to 30 kDa, including any intermediate values and subranges therebetween. In some embodiments, the molecular weight is in a range of from 3 to 20 kDa, including any intermediate values and subranges therebetween. In some embodiments, the molecular weight is in a range of from 3 to 10 kDa, including any intermediate values and subranges therebetween. In some embodiments, the molecular weight is in a range of from 3 to 7.5 kDa, including any intermediate values and subranges therebetween. In some embodiments, the molecular weight is in a range of from 3 to 5 kDa, including any intermediate values and subranges therebetween.

In some embodiments of any one of the embodiments described herein, the therapeutically active agent has a molecular weight of at least 4 kDa. In some embodiments, the molecular weight is in a range of from 4 to 150 kDa, including any intermediate values and subranges therebetween. In some embodiments, the molecular weight is in a range of from 4 to 100 kDa, including any intermediate values and subranges therebetween. In some embodiments, the molecular weight is in a range of from 4 to 75 kDa, including any intermediate values and subranges therebetween. In some embodiments, the molecular weight is in a range of from 4 to 50 kDa, including any intermediate values and subranges therebetween. In some embodiments, the molecular weight is in a range of from 4 to 30 kDa, including any intermediate values and subranges therebetween. In some embodiments, the molecular weight is in a range of from 4 to 20 kDa, including any intermediate values and subranges therebetween. In some embodiments, the molecular weight is in a range of from 4 to 10 kDa, including any intermediate values and subranges therebetween. In some embodiments, the molecular weight is in a range of from 4 to 7.5 kDa, including any intermediate values and subranges therebetween. In some embodiments, the molecular weight is in a range of from 4 to 5 kDa, including any intermediate values and subranges therebetween.

In some embodiments of any one of the embodiments described herein, the therapeutically active agent has a molecular weight of at least 5 kDa. In some embodiments, the molecular weight is in a range of from 5 to 150 kDa, including any intermediate values and subranges therebetween. In some embodiments, the molecular weight is in a range of from 5 to 100 kDa, including any intermediate values and subranges therebetween. In some embodiments, the molecular weight is in a range of from 5 to 75 kDa, including any intermediate values and subranges therebetween. In some embodiments, the molecular weight is in a range of from 5 to 50 kDa, including any intermediate values and subranges therebetween. In some embodiments, the molecular weight is in a range of from 5 to 30 kDa, including any intermediate values and subranges therebetween. In some embodiments, the molecular weight is in a range of from 5 to 20 kDa, including any intermediate values and subranges therebetween. In some embodiments, the molecular weight is in a range of from 5 to 10 kDa, including any intermediate values and subranges therebetween. In some embodiments, the molecular weight is in a range of from 5 to 7.5 kDa, including any intermediate values and subranges therebetween.

In some embodiments of any one of the embodiments described herein, the therapeutically active agent has a molecular weight of at least 10 kDa. In some embodiments, the molecular weight is in a range of from 10 to 150 kDa, including any intermediate values and subranges therebetween. In some embodiments, the molecular weight is in a range of from 10 to 100 kDa, including any intermediate values and subranges therebetween. In some embodiments, the molecular weight is in a range of from 10 to 75 kDa, including any intermediate values and subranges therebetween. In some embodiments, the molecular weight is in a range of from 10 to 50 kDa, including any intermediate values and subranges therebetween. In some embodiments, the molecular weight is in a range of from 10 to 30 kDa, including any intermediate values and subranges therebetween. In some embodiments, the molecular weight is in a range of from 10 to 20 kDa, including any intermediate values and subranges therebetween.

In some embodiments of any one of the embodiments described herein, the therapeutically active agent has a molecular weight of at least 20 kDa. In some embodiments, the molecular weight is in a range of from 20 to 150 kDa, including any intermediate values and subranges therebetween. In some embodiments, the molecular weight is in a range of from 20 to 100 kDa, including any intermediate values and subranges therebetween. In some embodiments, the molecular weight is in a range of from 20 to 75 kDa, including any intermediate values and subranges therebetween. In some embodiments, the molecular weight is in a range of from 20 to 50 kDa, including any intermediate values and subranges therebetween. In some embodiments, the molecular weight is in a range of from 20 to 30 kDa, including any intermediate values and subranges therebetween.

In some embodiments of any one of the embodiments described herein, the therapeutically active agent has a molecular weight of at least 50 kDa. In some embodiments, the molecular weight is in a range of from 50 to 150 kDa, including any intermediate values and subranges therebetween. In some embodiments, the molecular weight is in a range of from 50 to 100 kDa, including any intermediate values and subranges therebetween. In some embodiments, the molecular weight is in a range of from 50 to 75 kDa, including any intermediate values and subranges therebetween.

Without being bound by any particular theory, it is believed that agents having a relatively high molecular weight (e.g., at least 0.5 kDa, at least 1 kDa, at least 2 kDa, at least 3 kDa, at least 4 kDa) tend to be less efficiently absorbed upon oral administration than relatively small molecules (e.g., molecules having a molecular weight of less than 0.5 kDa, or less than 1 kDa) and therefore, their absorption is particularly susceptible to enhancement by activity of an absorption enhancer (e.g., NAC or a salt thereof) according to any of the respective embodiments described herein.

Herein throughout, “Dalton” or “Da” is considered as equivalent to gram/mol, and kDa is considered as equivalent to 1,000 grams/mol.

In some embodiments of any one of the embodiments described herein, the therapeutically active agent is a polypeptide or nucleic acid.

Without being bound by any particular theory, it is believed that agents which are polypeptides or nucleic acids tend to be poorly absorbed upon oral administration, for example, due to their polarity and/or relatively large molecular weight; and therefore, their absorption is particularly susceptible to enhancement by activity of an absorption enhancer (e.g., NAC or a salt thereof) and to increasing absorption enhancer activity by an alkaline group-containing polymer and a proton-accepting non-polymeric agent according to any of the respective embodiments described herein. It is further believed that absorption of polypeptides is further limited by degradation by pH-dependent activity of enzymes such as pepsin, and that a pH increase associated with an alkaline group-containing polymer can enhance absorption of a polypeptide by reducing pepsin activity (in addition to increasing absorption enhancer activity, as described herein).

In some embodiments of any one of the embodiments described herein, the therapeutically active agent is a polypeptide.

In some embodiments of any one of the embodiments described herein, the therapeutically active agent is a polypeptide having a molecular weight as described herein in any of the respective embodiments and any combination thereof.

In some embodiments of any one of the embodiments described herein, the therapeutically active agent is a hormone and/or cytokine (e.g., a hormone), an enzyme, and/or an antigen (e.g., wherein the composition serves as a vaccine). In some embodiments, the hormone and/or cytokine is a polypeptide hormone and/or cytokine, or an analog thereof.

Herein, an “analog” of a given polypeptide or nucleic acid encompasses various derivatives (e.g., synthetic analogs) of the polypeptide/nucleic acid, such as homologs (as defined herein), fragments (including fragments of a homolog) and substituted derivatives (e.g., comprising a substituent for enhancing stability and/or half-life) of the polypeptide/nucleic acid or homolog and/or fragment thereof. Preferably, the analog exhibits a biological activity of the polypeptide/nucleic acid.

Examples of polypeptides which may be utilized (per se or as analogs thereof) as therapeutically active agents according to embodiments of the invention include, without limitation, an antibody (e.g., monoclonal antibody), an antipathogenic peptide such as an antibacterial peptide (e.g., bacitracin, boceprevir, dalbavancin, daptomycin, enfuvirtide, oritavancin, teicoplanin, telaprevir, telavancin, vancomycin, and guavanin) and an antiviral peptide (e.g. bulevirtide), a blood clotting factor, a C-type natriuretic peptide (including derivatives thereof such as vosiritide), a calcitonin, disitertide, an endomorphin (e.g., endomorphin-1 and/or, endomorphin-2), an erythropoietin (e.g., darbepoetin), etelcalcitide, a gastric inhibitory polypeptide (GIP) or other GIP receptor agonist (e.g. tirzepatide), a glucagon or other glucagon receptor agonist, a GLP-1 (glucagon-like peptide-1, including derivatives thereof such as liraglutide, semaglutide, taspoglutide, albiglutide and dulaglutide), a GLP-1 receptor agonist (including GLP-1 receptor/glucagon receptor dual agonists) such as exendin-4 (including exenatide and lixisenatide), a GLP-2 (glucagon-like peptide-2, including derivatives thereof such as teduglutide, apraglutide and glepaglutide) or other GLP-2 receptor agonist (including GLP-2 receptor/glucagon receptor or GLP-2 receptor/GLP-1 receptor dual agonists such as dapiglutide, or GLP-2 receptor/GLP-1 receptor/glucagon receptor triple agonists), oxyntomodulin, a glucocerebrosidase, a gonadotropin (e.g., a follicle stimulating hormone, including derivatives thereof such as corifollitropin alfa), a gonadotropin releasing hormone (GnRH) or other GnRH receptor agonist (e.g., leuprorelin, buserelin, histrelin, goserelin, deslorelin, nafarelin and triptorelin), a GnRH receptor antagonist (e.g., degarelix, acyline, cetrorelix and ganirelix), a granulocyte-colony stimulating factor (G-CSF, e.g., filgrastim), a growth factor (e.g., insulin-like growth factor 1 (IGF-1), fibroblast growth factor (FGF), activity-dependent neurotrophic factor (including davunetide), and/or ciliary neurotrophic factor (including axokine, a homolog of a fragment of ciliary neurotrophic factor)), a growth hormone (e.g., somatotropin (a.k.a. growth hormone 1) and/or growth hormone 2 and/or the long acting acylated somapacitan), a growth hormone-inhibiting hormone, a.k.a. somatostatin or other somatostatin receptor agonist (including octreotide, romurtide, lanreotide, and pasireotide), a growth hormone-releasing hormone (including sermorelin,, CJC-1293 and CJC-1295), icatibant, insulin, an interferon, an interleukin, a kappa opioid receptor agonist such as a peripherally-restricted kappa opioid receptor agonist (e.g., difelikefalin, CR665, JT09 and dynorphins), a leptin, an oxytocin or other oxytocin receptor agonist, a melanocortin or other melanocortin receptor agonist (e.g., a melanocyte-stimulating hormone, afamelanotide, bremelanotide, melanotan II, modimelanotide, setmelanotide, alsactide, and tetracosactide), a motilin, an omentin, a parathyroid hormone, a parathyroid hormone related protein (including abaloparatide, a homolog of a parathyroid hormone related protein fragment, a parathyroid hormone, and a long acting analog Eneboparatide (AZP-3601)), a peptide YY, a pituitary adenylate cyclase-activating peptide (PACAP), thymopentin, a thymosin (e.g., thymosin a1), a TNF inhibitor (e.g., infliximab, adalimumab, certolizumab, golimumab and/or etanercept), a vasoactive intestinal peptide (e.g., aviptadil), and a vasopressin (including derivatives thereof such as desmopressin). Insulin, glucagon, GLP-1, GLP-2, parathyroid hormone, parathyroid hormone related protein, erythropoietin, calcitonin, gastric inhibitory polypeptide, gonadotropin releasing hormone, growth hormone-inhibiting hormone, melanocortins, motilin, leptin, peptide YY, vasopressin, vasoactive intestinal peptide, pituitary adenylate cyclase-activating peptide (PACAP), growth hormones, growth factors, and G-CSF are non-limiting examples of polypeptide hormones. Interferons, interleukins, erythropoietin, omentin and G-CSF are non-limiting examples of polypeptide cytokines.

According to some of any of the embodiments described herein, the polypeptide is selected from an antibody, an antipathogenic peptide, a blood clotting factor, a C-type natriuretic peptide, a calcitonin, disitertide, an endomorphin, an erythropoietin, a glucagon, a glucagon receptor agonist, a GLP-1 (glucagon-like peptide-1), a GLP-1 receptor agonist (including GLP-1 receptor/glucagon receptor dual agonist), a gastric inhibitory polypeptide (GIP), a gastric inhibitory polypeptide receptor agonist (including a GIP receptor/GLP-1 receptor dual agonist and glucagon receptor/GIP receptor/GLP-1 receptor triple agonist), a GLP-2 (glucagon-like peptide-2), a GLP-2 receptor agonist (including GLP-2 receptor/glucagon receptor dual agonist, GLP-2 receptor/GLP-1 receptor dual agonist or GLP-2 receptor/GIP receptor dual agonist), oxyntomodulin, a glucocerebrosidase, a gonadotropin, a gonadotropin releasing hormone, a gonadotropin releasing hormone receptor agonist, a gonadotropin releasing hormone receptor antagonist, a granulocyte-colony stimulating factor, a growth factor, a growth hormone, a somatostatin, a somatostatin receptor agonist, somapacitan, a growth hormone-releasing hormone, icatibant, an anti-viral peptide (e.g., bulevirtide), insulin, an interferon, an interleukin, a kappa opioid receptor agonist (e.g., a peripherally-restricted kappa opioid receptor agonist), a leptin, an oxytocin, an oxytocin receptor agonist, a melanocortin, a melanocortin receptor agonist, a motilin, an omentin, a parathyroid hormone or a fragment thereof or a long-acting analog thereof, a parathyroid hormone related protein, a peptide YY, a pituitary adenylate cyclase-activating peptide (PACAP), thymopentin, a thymosin, a TNF inhibitor, a vasoactive intestinal peptide, and a vasopressin, and analogs thereof.

According to some of any of the embodiments described herein, the polypeptide has a relatively long plasma half-life, due, for example, the presence of functional groups that have high affinity to plasma albumin, as, for example, in acylated peptides. Such polypeptides are also referred to herein as long-acting polypeptides or as long-acting analogs. Exemplary long-acting s polypeptides include like semaglutide, liraglutide, tirzepatide, Eneboparatide (AZP-3601) and somapacitan.

Therapeutically active agents according to embodiments of the invention may optionally exhibit more than one activity described herein; for example, a GLP-1 receptor agonist may optionally be a GLP-1 receptor/GLP-2 receptor dual agonist (e.g., dapiglutide), a GIP receptor/GLP-1 receptor dual agonist and/or a glucagon receptor/GIP receptor/GLP-1 receptor triple agonist, and a GLP-2 receptor agonist may optionally be a glucagon receptor/GLP-2 receptor dual agonist and/or a GIP receptor/GLP-2 receptor dual agonist.

An antibody according to any of the respective embodiments described herein any optionally be a single-domain antibody.

Herein and in the art, a “single-domain antibody” (also known in the art as a “nanobody”) refers to an antibody fragment consisting of a monomeric variable antibody domain (e.g., as opposed to a typical antibody structure which comprises four polypeptide chains, each of which comprise a variable antibody domain). Single-domain antibodies may optionally be prepared by reproducing a single variable domain (e.g., by recombinant protein technology) of a more conventional antibody (e.g., 4-chain antibody) or of a heavy-chain antibody from a camelid or a cartilaginous fish (e.g., shark).

Without being bound by any particular theory, it is believed that the molecular weight of the single-domain antibody (typically about 12 kDa to about 15 kDa) is particularly suitable for oral administration using an absorption enhancer (according to any of the embodiments described herein).

In some embodiments of any one of the embodiments described herein, the therapeutically active agent is parathyroid hormone (PTH) or a fragment thereof.

Herein, the term “parathyroid hormone” or its abbreviation “PTH” encompasses parathyroid hormone (having a naturally occurring amino acid sequence, e.g., an 84-amino acid sequence in humans) and fragments and/or homologs and/or long acting derivatives or analogs of the parathyroid hormone.

Teriparatide is an example of a parathyroid hormone fragment, composed of amino acids 1-34 (i.e., an N-terminal portion) of the full human parathyroid hormone polypeptide. The term “teriparatide” is used interchangeably herein with the terms “hPTH (1-34)” and “human parathyroid hormone (1-34)”.

Eneboparatide (AZP-3601) is an example of a parathyroid hormone long-acting analog.

Herein, for the sake of brevity, the term “parathyroid hormone” or its abbreviation “PTH” encompasses parathyroid hormone (having a naturally occurring amino acid sequence, e.g., in humans), fragments thereof and homologs or analogs (e.g., long-acting) of the parathyroid hormone or the fragment thereof, except where indicated otherwise.

Examples of nucleic acids which may be utilized (per se or as analogs thereof) as therapeutically active agents according to embodiments of the invention include, without limitation, antisense nucleic acids (e.g., milasen, eteplirsen, fomiversen, golodirsen, inotersen, mipomersen, nusinersen, tofersen, tominersen, viltolarsen, and volanesorsen), small interfering RNA (e.g., givosiran, inclisiran, lumasiran, and patisiran), micro RNA, RNA aptamers (e.g., pegapatanib), messenger RNA (e.g., of an mRNA vaccine), and DNA (e.g., of a DNA vaccine).

It has been reported that therapeutically active agents which exhibit more than one of the following criteria tend to be poorly absorbed upon oral administration (when administered alone), a phenomenon referred to in the art as “Lipinski's rule of 5”:
(i) a total number of nitrogen-hydrogen bonds and oxygen hydrogen bonds (which are typically hydrogen bond donors) which is more than 5;
(ii) a total number of nitrogen and oxygen atoms (which are typically hydrogen bond acceptors) which is more than 5;
(iii) an octanol-water partition coefficient (log P) which is greater than 5; and/or
(iv) a molecular weight of at least 500 Da (0.5 kDa).

The abovementioned criteria (i) and (ii) are associated with hydrogen bonding and hydrophilicity; whereas criteria (iii) is associated with lipophilicity.

As described herein, therapeutically active agents poorly absorbed upon oral administration when administered alone are particularly suitable for being included in compositions as described herein, in order to enhance their absorption.

In some embodiments of any one of the embodiments described herein, the therapeutically active agent meets at least one of the abovementioned criteria (i), (ii), (iii) and (iv). In some embodiments, the therapeutically active agent meets at least two of the abovementioned criteria (i), (ii), (iii) and (iv). In some embodiments, the therapeutically active agent meets at least three of the abovementioned criteria (i), (ii), (iii) and (iv). In some embodiments, the therapeutically active agent meets all four of the abovementioned criteria (i), (ii), (iii) and (iv).

In some embodiments of any one of the embodiments described herein, the therapeutically active agent has a molecular weight of at least 0.5 kDa, in accordance with any one of the embodiments described herein relating to a molecular weight of at least 0.5 kDa, and further meets at least one of the abovementioned criteria (i), (ii) and (iii). In some such embodiments, the therapeutically active agent meets at least two of the abovementioned criteria (i), (ii) and (iii).

Dihydroergotamine and fondaparinux are non-limiting examples of non-peptidic agents having a molecular weight of at least 0.5 kDa, which are poorly absorbed upon oral administration.

In some embodiments of any one of the embodiments described herein, the therapeutically active agent has a molecular weight of less than 0.5 kDa, and meets at least one of the abovementioned criteria (i), (ii) and (iii). In some such embodiments, the therapeutically active agent meets at least two of the abovementioned criteria (i), (ii) and (iii). In some such embodiments, the therapeutically active agent meets all three of the abovementioned criteria (i), (ii) and (iii).

In addition, ionic molecules tend to be poorly absorbed upon oral administration, generally due to a considerably reduced ability to cross lipid membranes. Whether a molecule is ionic or non-ionic often depends on pH, which varies according to location in the gastrointestinal tract. In general, it is believed that the more a therapeutically active agent is in ionic form in the gastrointestinal tract, the more likely it is to be poorly absorbed upon oral administration.

In some embodiments of any one of the embodiments described herein, the therapeutically active agent is ionic in an aqueous solution at a pH of 7.0.

In some embodiments of any one of the embodiments described herein, the therapeutically active agent is ionic in an aqueous solution at a pH of 6.0.

In some embodiments of any one of the embodiments described herein, the therapeutically active agent is ionic in an aqueous solution at a pH of 5.0.

In some embodiments of any one of the embodiments described herein, the therapeutically active agent is ionic in an aqueous solution at a pH of 4.0.

In some embodiments of any one of the embodiments described herein, the therapeutically active agent is ionic in an aqueous solution at a pH of 3.0.

In some embodiments of any one of the embodiments described herein, the therapeutically active agent is ionic in an aqueous solution at a pH of 2.0.

In some embodiments of any one of the embodiments described herein, the therapeutically active agent is ionic in an aqueous solution at a pH of 1.0.

Examples of such agents include, without limitation, compounds comprising at least one basic group (e.g., amine group) which is positively charged at a pH of 7.0 (or less).

Herein, a compound is considered “ionic” when it comprises at least one functional group which is charged in at least 50% of the molecules in a population of molecules of the compound under designated conditions (e.g., in an aqueous solution at a designated pH value or range of pH values). The skilled person will be readily capable of determining whether a functional group is charged in at least 50% of the molecules, for example, by determining a pKa value associated with the functional group. An ionic compound, as defined herein, may optionally have a net negative charge, optionally a net positive charge, and optionally an equal number of negatively charged functional groups and positively functional groups, resulting in no net charge.

In some embodiments of any one of the embodiments described herein, the therapeutically active agent is ionic in an aqueous solution at all pH values within a range of from 5.0 to 7.0. In some embodiments, the therapeutically active agent is ionic in an aqueous solution at all pH values within a range of from 5.0 to 8.0. In some embodiments, the therapeutically active agent is ionic in an aqueous solution at all pH values within a range of from 4.0 to 9.0. In some embodiments, the therapeutically active agent is ionic in an aqueous solution at all pH values within a range of from 3.0 to 10.0. In some embodiments, the therapeutically active agent is ionic in an aqueous solution at all pH values within a range of from 2.0 to 11.0.

In some embodiments of any one of the embodiments described herein, the therapeutically active agent is ionic at a pH value and/or range according to any one of the abovementioned embodiments, and further has a molecular weight of at least 0.5 kDa, in accordance with any one of the embodiments described herein relating to a molecular weight of at least 0.5 kDa.

In some embodiments of any one of the embodiments described herein, the therapeutically active agent is ionic at a pH value and/or range according to any one of the abovementioned embodiments, and further has a molecular weight of less than 0.5 kDa.

Examples of ionic therapeutically active agents which tend to have a molecular weight of less than 0.5 kDa, and which tend to exhibit poor absorption upon oral administration, include, without limitation, bisphosphonates (e.g., for use in treating osteoporosis and related conditions) such as alendronate, clodronate, etidronate, ibandronate, neridronate, olpadronate, pamidronate, risedronate, tiludronate and zoledronate; and cromolyn (e.g., cromolyn sodium).

When the therapeutically active agent is a polypeptide, it is in ionic form in accordance with its isoelectric point (pl).

Herein and in the art, an isoelectric point (pl) of a polypeptide is the pH at which the protein carries no net electrical charge, that is, is in a non-ionic form. At this point, the number of positively charged groups equals the number of negatively charged groups, making the protein electrically neutral. The pI of a polypeptide can be experimentally determined or calculated based on the amino acid sequence of the protein, taking into account the pKa values of its ionizable groups (such as the side chains of acidic and basic amino acids).

According to some of any of the embodiments described herein, the therapeutically active agent is a polypeptide featuring pI of at least 1.0, or of at least 2.0, or of at least 3.0, or of at least 4.0, or of at least 5.0, or of at least 6.0, or of at least 7.0, or of at least 8.0, or of at least 8.5, or of at least 9.0, or higher. According to some of any of the embodiments described herein, the therapeutically active agent is a polypeptide featuring pI higher than 8.0, for example, from 8.0 to 14.0, or from 8.0 to 12.0, or from 8.0 to 10.0, or from 8.5 to 10.0.

In some embodiments of any one of the embodiments described herein, the therapeutically active agent is a Class III agent according to the Biopharmaceutics Classification System (BCS), as provided by the U.S. FDA, that is, the therapeutically active agent is characterized by low permeability and high solubility.

In the context of the BCS, the phrase “low permeability” refers herein and in the art to absorption of less than 90% of a given agent upon oral administration in humans (in the absence of absorption enhancer), as determined by mass-balance determination and/or in comparison to an intravenous dose.

In some embodiments, absorption of a Class III therapeutically active agent is less than 50% upon oral administration (in the absence of absorption enhancer). In some embodiments, absorption is less than 20% upon oral administration (in the absence of absorption enhancer). In some embodiments, absorption is less than 10% upon oral administration (in the absence of absorption enhancer). In some embodiments, absorption is less than 5% upon oral administration (in the absence of absorption enhancer). In some embodiments, absorption is less than 2% upon oral administration (in the absence of absorption enhancer). In some embodiments, absorption is less than 1% upon oral administration (in the absence of absorption enhancer). In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In the context of the BCS, the phrase “high solubility” refers herein and in the art to an amount of therapeutically active agent in an administered dose being soluble in 250 ml or less of water over a pH range of 1 to 7.5.

In some of any of the embodiments described herein, the composition is a unit dosage form composition, and the unit dosage form comprises at least 50 μg of therapeutically active agent. In some embodiments, the unit dosage form comprises at least 100 μg of therapeutically active agent. In some embodiments, the unit dosage form comprises at least 200 μg of therapeutically active agent. In some embodiments, the unit dosage form comprises at least 500 μg of therapeutically active agent. In some embodiments, the amount of absorption enhancer in the unit dosage form is in accordance with any one of the ratios of absorption enhancer to therapeutically active agent described herein. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the embodiments described herein, the composition is a unit dosage form composition, and the unit dosage form comprises 3000 μg or less of therapeutically active agent. In some embodiments, the unit dosage form comprises 2000 μg or less of therapeutically active agent. In some embodiments, the unit dosage form comprises 1000 μg or less of therapeutically active agent. In some embodiments, the amount of absorption enhancer in the unit dosage forms is in accordance with any one of the ratios of absorption enhancer to therapeutically active agent described herein. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

In some of any of the embodiments described herein, the composition is a unit dosage form composition, and the unit dosage form comprises from 100 to 100000 μg, or from 100 to 50000 μg, or from 100 to 30000 μg, or from 100 to 10000 μg, 100 to 8000 μg, 100 to 5000 μg 100 to 3000 μg of therapeutically active agent, including any intermediate values and subranges therebetween. In some embodiments, the unit dosage form comprises from 200 to 2000 μg of therapeutically active agent, including any intermediate values and subranges therebetween. In some embodiments, the unit dosage form comprises from 500 to 1000 μg of therapeutically active agent, including any intermediate values and subranges therebetween. In some embodiments, the unit dosage form comprises from 1000 to 3000 μg of therapeutically active agent, including any intermediate values and subranges therebetween. In some embodiments, the unit dosage form comprises from 1500 to 2500 μg of therapeutically active agent, including any intermediate values and subranges therebetween. In some embodiments, the therapeutically active agent is a parathyroid hormone or a fragment thereof. In some embodiments, the therapeutically active agent is teriparatide. In some embodiments, the amount of absorption enhancer in the unit dosage forms is in accordance with any one of the ratios of absorption enhancer to therapeutically active agent described herein. In some of the aforementioned embodiments, the absorption enhancer is NAC (8-N-(2-hydroxybenzoyl)aminocaprylic acid) or a salt thereof (e.g., sodium 8-N-(2-hydroxybenzoyl)aminocaprylate).

Exemplary therapeutically active agents usable in the context of the present embodiments are described in the Examples section that follows.

Exemplary compositions according to exemplary embodiments are described in the Examples section that follows.

In some embodiments of any one of the embodiments described herein, when the therapeutically active agents is a polypeptide, the composition further comprises at least one protease inhibitor, for example, a type of protease inhibitor, a concentration and/or absolute amount of protease inhibitor, and/or a ratio of protease inhibitor to absorption enhancer and/or therapeutically active agent according to any one of the embodiments relating to a protease inhibitor described in any of U.S. Patent Application Publication No. 2011/0142800, and International Patent Application Publications WO 2016/128972 and WO 2018/033927, the contents of each of which (especially contents relating to protease inhibitors) are incorporated herein.

Herein throughout, the term “protease inhibitor” refers to a compound which reduces a proteolytic activity of a protease, for example, a proteolytic activity which inactivates a therapeutically active agent described herein. The term “protease inhibitor” encompasses, for example, both large molecules (e.g., proteins) and small molecules, as well as both naturally occurring compounds and synthetic compounds.

In some embodiments of any of the embodiments described herein, the at least one protease inhibitor comprises at least one trypsin inhibitor. In some embodiments, the at least one protease inhibitor consists essentially of one or more trypsin inhibitor(s).

Examples of trypsin inhibitors which may be utilized in any one of the embodiments described herein include, without limitation, lima bean trypsin inhibitor, aprotinin, soybean trypsin inhibitor, ovomucoid trypsin inhibitor and any combination thereof. In some embodiments, the at least one trypsin inhibitor comprises soybean trypsin inhibitor (SBTI). In some embodiments, the at least one trypsin inhibitor (an optionally the at least one protease inhibitor) consists essentially of SBTI.

In some embodiments of any of the embodiments described herein, the at least one protease inhibitor comprises at least one serpin. In some embodiments, the at least one protease inhibitor consists essentially of one or more serpin(s).

Examples of serpins which may be utilized in any one of the embodiments described herein, include, without limitation, alpha 1-antitrypsin, antitrypsin-related protein, alpha 1-antichymotrypsin, kallistatin, protein C inhibitor, cortisol binding globulin, thyroxine-binding globulin, angiotensinogen, centerin, protein Z-related protease inhibitor, vaspin, monocyte/neutrophil elastase inhibitor, plasminogen activator inhibitor-2, squamous cell carcinoma antigen-1 (SCCA-1), squamous cell carcinoma antigen-2 (SCCA-2), maspin, proteinase inhibitor 6 (PI-6), megsin, serpin B8 (PI-8), serpin B9 (PI-9), bomapin, yukopin, hurpin/headpin, antithrombin, heparin cofactor II, plasminogen activator inhibitor 1, glia-derived nexin, pigment epithelium derived factor, alpha 2-antiplasmin, complement 1-inhibitor, 47 kDa heat shock protein (HSP47), neuroserpin and pancpin.

In some embodiments of any of the embodiments described herein, the at least one protease inhibitor comprises at least one cysteine protease inhibitor. In some embodiments, the at least one protease inhibitor consists essentially of one or more cysteine protease inhibitor(s).

Examples of cysteine protease inhibitors which may be utilized in any one of the embodiments described herein include, without limitation, type 1 cystatins, type 2 cystatins, human cystatins C, D, S, SN, and SA, cystatin E/M, cystatin F, and type 3 cystatins (including kininogens).

In some embodiments of any of the embodiments described herein, the at least one protease inhibitor comprises at least one threonine protease inhibitor. In some embodiments, the at least one protease inhibitor consists essentially of one or more threonine protease inhibitor(s).

Examples of threonine protease inhibitors which may be utilized in any one of the embodiments described herein include, without limitation, bortezomib, MLN-519, ER-807446 and TMC-95A.

In some embodiments of any of the embodiments described herein, the at least one protease inhibitor comprises at least one aspartic protease inhibitor. In some embodiments, the at least one protease inhibitor consists essentially of one or more aspartic protease inhibitor(s).

Examples of aspartic protease inhibitors which may be utilized in any one of the embodiments described herein, include, without limitation, α₂-macroglobulin, pepstatin A, aspartic protease inhibitor 11, aspartic protease inhibitor 1, aspartic protease inhibitor 2, aspartic protease inhibitor 3, aspartic protease inhibitor 4, aspartic protease inhibitor 5, aspartic protease inhibitor 6, aspartic protease inhibitor 7, aspartic protease inhibitor 8, aspartic protease inhibitor 9, pepsin inhibitor Dit33, and protease A inhibitor 3.

In some embodiments of any of the embodiments described herein, the at least one protease inhibitor comprises at least one metalloprotease inhibitor. In some embodiments, the at least one protease inhibitor consists essentially of one or more metalloprotease inhibitor(s).

Examples of metalloprotease inhibitors which may be utilized in any one of the embodiments described herein, include, without limitation, angiotensin-1-converting enzyme inhibitory peptide, antihemorrhagic factor BJ46a, beta-casein, proteinase inhibitor CeKI, venom metalloproteinase inhibitor DM43, carboxypeptidase A inhibitor, smpI, IMPI, alkaline proteinase, latexin, carboxypeptidase inhibitor, antihemorrhagic factor HSF, testican-3, SPOCK3, TIMP1, metalloproteinase inhibitor 1, metalloproteinase inhibitor 2, TIMP2, metalloproteinase inhibitor 3, TIMP3, metalloproteinase inhibitor 4, TIMP4, putative metalloproteinase inhibitor tag-225, tissue inhibitor of metalloprotease, WAP, kazal inhibitor, immunoglobulin, and kunitz and NTR domain-containing protein 1.

Examples of protease inhibitors which may be utilized in any one of the embodiments described herein also include, without limitation, AEBSF-HCl, ε-aminocaproic acid, α1-antichymotypsin, antipain, antithrombin III, al-antitrypsin, APMSF (4-amidinophenyl-methane sulfonyl-fluoride), sprotinin, benzamidine, chymostatin, DFP (diisopropylfluoro-phosphate), leupeptin, 4-(2-Aminoethyl)-benzenesulfonyl fluoride hydrochloride, PMSF (phenylmethyl sulfonyl fluoride), TLCK (1-chloro-3-tosylamido-7-amino-2-heptanone), TPCK (1-chloro-3-tosylamido-4-phenyl-2-butanone), pentamidine isothionate, pepstatin, guanidium, α2-macroglobulin, a chelating agent of zinc, and iodoacetate.

In some embodiments of any one of the embodiments described herein, the amount of a protease inhibitor in a composition or a unit dosage form described herein is at least about 0.1 mg. In some embodiments, the amount of a protease inhibitor in a composition or a unit dosage form described herein is at least about 0.2 mg. In some embodiments, the amount of a protease inhibitor in a composition or a unit dosage form described herein is at least about 0.3 mg. In some embodiments, the amount of a protease inhibitor in a composition or a unit dosage form described herein is at least about 0.4 mg. In some embodiments, the amount of a protease inhibitor in a composition or a unit dosage form described herein is at least about 0.6 mg. In some embodiments, the amount of a protease inhibitor in a composition or a unit dosage form described herein is at least about 0.8 mg. In some embodiments, the amount of a protease inhibitor in a composition or a unit dosage form described herein is at least about 1 mg. In some embodiments, the amount of a protease inhibitor in a composition or a unit dosage form described herein is at least about 1.5 mg. In some embodiments, the amount of a protease inhibitor in a composition or a unit dosage form described herein is at least about 2 mg. In some embodiments, the amount of a protease inhibitor in a composition or a unit dosage form described herein is at least about 2.5 mg. In some embodiments, the amount of a protease inhibitor in a composition or a unit dosage form described herein is at least about 3 mg. In some embodiments, the amount of a protease inhibitor in a composition or a unit dosage form described herein is at least about 5 mg. In some embodiments, the amount of a protease inhibitor in a composition or a unit dosage form described herein is at least about 7 mg. In some embodiments, the amount of a protease inhibitor in a composition or a unit dosage form described herein is at least about 10 mg. In some embodiments, the amount of a protease inhibitor in a composition or a unit dosage form described herein is at least about 12 mg. In some embodiments, the amount of a protease inhibitor in a composition or a unit dosage form described herein is at least about 15 mg. In some embodiments, the amount of a protease inhibitor in a composition or a unit dosage form described herein is at least about 20 mg. In some embodiments, the amount of a protease inhibitor in a composition or a unit dosage form described herein is at least about 30 mg. In some embodiments, the amount of a protease inhibitor in a composition or a unit dosage form described herein is at least about 50 mg. In some embodiments, the amount of a protease inhibitor in a composition or a unit dosage form described herein is at least about 70 mg. In some embodiments, the amount of a protease inhibitor in a composition or a unit dosage form described herein is at least about 100 mg.

In some embodiments of any one of the embodiments described herein, the amount of a protease inhibitor in a composition or a unit dosage form described herein is in a range of from 0.1 to 1 mg. In some embodiments, the amount of a protease inhibitor in a composition or a unit dosage form described herein is in a range of from 0.2 to 1 mg. In some embodiments, the amount of a protease inhibitor in a composition or a unit dosage form described herein is in a range of from 0.3 to 1 mg. In some embodiments, the amount of a protease inhibitor in a composition or a unit dosage form described herein is in a range of from 0.5 to 1 mg.

In some embodiments of any one of the embodiments described herein, the amount of a protease inhibitor in a composition or a unit dosage form described herein is in a range of from 0.1 to 2 mg. In some embodiments, the amount of a protease inhibitor in a composition or a unit dosage form described herein is in a range of from 0.2 to 2 mg. In some embodiments, the amount of a protease inhibitor in a composition or a unit dosage form described herein is in a range of from 0.3 to 2 mg. In some embodiments, the amount of a protease inhibitor in a composition or a unit dosage form described herein is in a range of from 0.5 to 2 mg. In some embodiments, the amount of a protease inhibitor in a composition or a unit dosage form described herein is in a range of from 1 to 2 mg.

In some embodiments of any one of the embodiments described herein, the amount of a protease inhibitor in a composition or a unit dosage form described herein is in a range of from 1 to 10 mg. In some embodiments, the amount of a protease inhibitor in a composition or a unit dosage form described herein is in a range of from 2 to 10 mg. In some embodiments, the amount of a protease inhibitor in a composition or a unit dosage form described herein is in a range of from 3 to 10 mg. In some embodiments, the amount of a protease inhibitor in a composition or a unit dosage form described herein is in a range of from 5 to 10 mg.

In some embodiments of any one of the embodiments described herein, the amount of a protease inhibitor in a composition or a unit dosage form described herein is in a range of from 1 to 20 mg. In some embodiments, the amount of a protease inhibitor in a composition or a unit dosage form described herein is in a range of from 2 to 20 mg. In some embodiments, the amount of a protease inhibitor in a composition or a unit dosage form described herein is in a range of from 3 to 20 mg. In some embodiments, the amount of a protease inhibitor in a composition or a unit dosage form described herein is in a range of from 5 to 20 mg. In some embodiments, the amount of a protease inhibitor in a composition or a unit dosage form described herein is in a range of from to 20 mg.

10 In some embodiments of any one of the embodiments described herein, the amount of a protease inhibitor in a composition or a unit dosage form described herein is in a range of from 10 to 100 mg. In some embodiments, the amount of a protease inhibitor in a composition or a unit dosage form described herein is in a range of from 20 to 100 mg. In some embodiments, the amount of a protease inhibitor in a a composition or unit dosage form described herein is in a range of from 30 to 100 mg. In some embodiments, the amount of a protease inhibitor in a composition or a unit dosage form described herein is in a range of from 50 to 100 mg.

In some embodiments of any one of the embodiments described herein, the amount of a protease inhibitor in a composition or a unit dosage form described herein is in a range of from 10 to 200 mg. In some embodiments, the amount of a protease inhibitor in a composition or a unit dosage form described herein is in a range of from 20 to 200 mg. In some embodiments, the amount of a protease inhibitor in a composition or a unit dosage form described herein is in a range of from 30 to 200 mg. In some embodiments, the amount of a protease inhibitor in a composition or a unit dosage form described herein is in a range of from 50 to 200 mg. In some embodiments, the amount of a protease inhibitor in a composition or a unit dosage form described herein is in a range of from 100 to 200 mg.

In some embodiments of any one of the embodiments described herein, the amount of a protease inhibitor in a composition or a unit dosage form described herein is at least about 10 kallikrein inactivator units (k.i.u.). In some embodiments, the amount of a protease inhibitor in a composition or a unit dosage form described herein is at least about 12 k.i.u. In some embodiments, the amount of a protease inhibitor in a composition or a unit dosage form described herein is at least about 15 k.i.u. In some embodiments, the amount of a protease inhibitor in a composition or a unit dosage form described herein is at least about 20 k.i.u. In some embodiments, the amount of a protease inhibitor in a composition or a unit dosage form described herein is at least about 30 k.i.u. In some embodiments, the amount of a protease inhibitor in a composition or a unit dosage form described herein is at least about 40 k.i.u. In some embodiments, the amount of a protease inhibitor in a composition or unit dosage form described herein is at least about 50 k.i.u. In some embodiments, the amount of a protease inhibitor in a composition or a unit dosage form described herein is at least about 70 k.i.u. In some embodiments, the amount of a protease inhibitor in a composition or a unit dosage form described herein is at least about 100 k.i.u. In some embodiments, the amount of a protease inhibitor in a composition or a unit dosage form described herein is at least about 150 k.i.u. In some embodiments, the amount of a protease inhibitor in a composition or a unit dosage form described herein is at least about 200 k.i.u. In some embodiments, the amount of a protease inhibitor in a composition or a unit dosage form described herein is at least about 300 k.i.u. In some embodiments, the amount of a protease inhibitor in a composition or a unit dosage form described herein is at least about 500 k.i.u. In some embodiments, the amount of a protease inhibitor in a composition or a unit dosage form described herein is at least about 700 k.i.u. In some embodiments, the amount of a protease inhibitor in a composition or a unit dosage form described herein is at least about 1000 k.i.u. In some embodiments, the amount of a protease inhibitor in a composition or a unit dosage form described herein is at least about 1500 k.i.u. In some embodiments, the amount of a protease inhibitor in a composition or a unit dosage form described herein is at least about 3000 k.i.u. In some embodiments, the amount of a protease inhibitor in a composition or a unit dosage form described herein is at least about 4000 k.i.u. In some embodiments, the amount of a protease inhibitor in a composition or a unit dosage form described herein is at least about 5000 k.i.u.

Herein and in the art, a “kallikrein inactivating unit” (k.i.u) refers to an amount of protease inhibitor that has the ability to inhibit 2 units of kallikrein by 50% (e.g., in aqueous solution at an optimal pH and solution volume for activity of the protease inhibitor).

In some embodiments of any one of the embodiments described herein, a weight ratio of protease inhibitor to therapeutically active agent is in a range of from 1:1 to 5:1 (protease inhibitor: therapeutically active agent). In some embodiments, a weight ratio of protease inhibitor to therapeutically active agent is in a range of from 5:1 to 10:1. In some embodiments, a weight ratio of protease inhibitor to therapeutically active agent is in a range of from 10:1 to 20:1. In some embodiments, a weight ratio of protease inhibitor to therapeutically active agent is in a range of from 20:1 to 30:1. In some embodiments, a weight ratio of protease inhibitor to therapeutically active agent is in a range of from 30:1 to 40:1. In some embodiments, a weight ratio of protease inhibitor to therapeutically active agent is in a range of from 40:1 to 50:1. In some embodiments, a weight ratio of protease inhibitor to therapeutically active agent is in a range of from 50:1 to 75:1. In some embodiments, a weight ratio of protease inhibitor to therapeutically active agent is in a range of from 75:1 to 100:1. In some embodiments, a weight ratio of protease inhibitor to therapeutically active agent is in a range of from 100:1 to 200:1. In some embodiments, a weight ratio of protease inhibitor to therapeutically active agent is in a range of from 200:1 to 300:1. In some embodiments, a weight ratio of protease inhibitor to therapeutically active agent is in a range of from 300:1 to 400:1. In some embodiments, a weight ratio of protease inhibitor to therapeutically active agent is in a range of from 400:1 to 500:1. In some embodiments, the protease inhibitor is soybean trypsin inhibitor.

According to some embodiments of any of the embodiments described herein, the composition is devoid of a protease inhibitor.

### Uses and Indications:

In some embodiments of any of the embodiments described herein, the composition according to any of the respective embodiments described herein is for use in the treatment of a condition treatable by the therapeutically active agent (according to any of the respective embodiments described herein), the treatment comprising oral administration of the composition.

According to an aspect of some embodiments of the invention, there is provided a method of treating a condition treatable by oral administration of a therapeutically active agent (according to any of the respective embodiments described herein) in a subject in need thereof, the method comprising orally administering to the subject a composition according to any of the respective embodiments described herein comprising the respective therapeutically active agent (according to any of the respective embodiments described herein).

The skilled person will be capable of determining which conditions are treatable by oral administration of any given therapeutically active agent described herein.

Examples of conditions treatable according to embodiments of the invention include, without limitation, hyperglycemia and/or diabetes (e.g., wherein the therapeutically active agent is an insulin-like growth factor, an insulin, a GLP-1 or GLP-1 receptor agonist, or another agent which reduces blood glucose levels); hypoglycemia (e.g., wherein the therapeutically active agent is a growth hormone-inhibiting hormone, glucagon, or another agent which increases blood glucose levels); osteoporosis and conditions associated with a bone fracture or bone defect (e.g., wherein the therapeutically active agent is a PTH, parathyroid hormone related protein, GLP-2 or GLP-2 receptor agonist, or calcitonin); hypoparathyroidism (e.g., wherein the therapeutically active agent is a PTH); obesity (e.g., wherein the therapeutically active agent is peptide YY, a ciliary neurotrophic factor, a GLP-1 or GLP-1 receptor agonist, a growth hormone-inhibiting hormone, or a growth hormone); osteoarthritis (e.g., wherein the therapeutically active agent is a GLP-1, GLP-1 receptor agonist and/or PTH) an inflammatory condition and/or autoimmune condition (e.g., wherein the therapeutically active agent is a TNF inhibitor, interferon, interleukin, growth hormone, or vasoactive intestinal peptide); pain and/or pruritus, such as uremic pruritus (e.g., wherein the therapeutically active agent is an endomorphin and/or kappa opioid receptor agonist); growth deficiency (e.g., wherein the therapeutically active agent is a growth hormone); hereditary angioedema (e.g., wherein the therapeutically active agent is icatibant); lipodystrophy (e.g., wherein the therapeutically active agent is a leptin or a growth hormone-releasing hormone); a fatty liver disease such as non-alcoholic steatohepatitis (e.g., wherein the therapeutically active agent is a growth hormone-releasing hormone); short bowel syndrome (e.g., wherein the therapeutically active agent is a GLP-2 or GLP-2 receptor agonist, or a growth hormone); a hormone or enzyme deficiency (e.g., wherein the therapeutically active agent is a hormone or enzyme described herein which is deficient); tissue damage (e.g., wherein the therapeutically active agent is a growth factor); bleeding due to tissue damage or a bleeding disorder such as hemophilia (e.g., wherein the therapeutically active agent is a blood clotting factor or vasopressin); anemia (e.g., wherein the therapeutically active agent is an erythropoietin); leukopenia (e.g., wherein the therapeutically active agent is a G-CSF); a proliferative disease or disorder such as cancer, endometriosis or acromegaly (e.g., wherein the therapeutically active agent is an interleukin, interferon, erythropoietin, growth hormone-inhibiting hormone, GnRH, GnRH receptor antagonist or disitertide); infertility or other condition treatable by in vitro fertilization (e.g., wherein the therapeutically active agent is a gonadotropin or gonadotropin receptor agonist, a GnRH or a GnRH receptor agonist, or a GnRH receptor antagonist); and an infection such as a bacterial or viral infection (e.g., wherein the therapeutically active agent is an antipathogenic peptide, an antibody or interferon).

Without being bound by any particular theory, it is believed that compositions described herein facilitate absorption in the stomach (rather than in the intestines), and that this is particularly advantageous in treating subjects in which intestinal function may be deficient, such as short bowel syndrome.

Examples of suitable conditions in which activity of growth hormone and/or analogs thereof include, without limitation, burns, fibromyalgia, growth hormone deficiency, heart failure, inflammatory and/or autoimmune conditions (e.g., Crohn's disease, ulcerative colitis and/or multiple sclerosis), muscle deterioration (e.g., wasting associated with AIDS), obesity, short bowel syndrome, and short stature and/or growth deficiency (e.g., severe idiopathic short stature or short stature associated with Tumer syndrome, chronic kidney failure, Prader-Willi syndrome, and/or intrauterine growth restriction).

Examples of suitable conditions in which activity of GHRH and/or analogs thereof, and/or inducing release of growth hormone, is beneficial, include, without limitation, cognitive impairment (e.g., associated with agent, such as mild cognitive impairment, with pre-Alzheimer's symptoms (pre-onset Alzheimer's) and/or with dementia); sleep impairment (e.g., age-related sleep impairment); a disease or disorder associated with fat accumulation and/or hypercholesterolemia; a catabolic/wasting condition; a growth hormone deficiency (e.g., for providing a growth hormone replacement therapy, and/or treating idiopathic short stature); a disease or disorder associated with immunodeficiency; a disease or disorder associated with elevated cholesterol level, non-HDL cholesterol level, triglyceride level and/or total cholesterol/HDL cholesterol ratio (e.g., for reducing the elevated level and/or ratio); a disease or disorder associated with lean body mass and/or elevated trunk fat, visceral fat, visceral adipose tissue, visceral adipose tissue/subcutaneous adipose tissue ratio and/or abdominal girth (e.g., for improving any of the aforementioned parameters); a decrease in female fertility (e.g., as part of a fertility treatment in women); a lactation failure; a GHRH receptor-related tumor; hypothalamic pituitary dwarfism; a wound and/or burn (e.g., for improving healing); a debilitating infection (acute or chronic); and a disease or disorder associated with deficient and/or decreased bone formation (e.g., osteoporosis).

Examples of diseases or disorders associated with fat accumulation and/or hypercholesterolemia include, without limitation, obesity (abdominal obesity, optionally with metabolic disorders and/or relative GH deficiency), abdominal adiposity, metabolic syndrome, lipohypertrophy, lipoatrophy, lipodystrophy (e.g., HIV-associated lipodystrophy), dyslipidemia, hypertriglyceridemia, non-alcoholic fatty liver, and non-alcoholic steatohepatitis.

Without being bound by any particular theory, it is believed that compositions comprising a GHRH (or analog thereof) according to any of the respective embodiments described herein can promote absorption for a brief period of time (e.g., by facilitating absorption briefly in the stomach), thereby resulting in a pulse-like pharmacokinetic profile. Such a pharmacokinetic profile can be advantageous in that it mimics the profile of GHRH injections.

A treatment according to any of the embodiments described herein (e.g., using GHRH or related agent) may optionally be for enhancing an immune reaction, for example, for treating a disease or disorder associated with immunodeficiency.

Examples of diseases or disorders associated with immunodeficiency include, without limitation, T-cell immunodeficiency, immunodeficiency associated with aging, immunodeficiency associated with HIV infection/AIDS, and immunodeficiency associated with high-dose chemotherapy and/or radiotherapy.

A treatment according to any of the embodiments described herein (e.g., using GHRH or related agent) may optionally be for enhancing anabolism (e.g., for example to increase muscle mass and/or function), for example, for treating a catabolic/wasting conditions.

Examples of catabolic/wasting conditions include, without limitation, acute or chronic renal failure (e.g., acute or chronic renal failure wasting), chronic heart failure (e.g., chronic heart failure wasting), chronic obstructive pulmonary disease (COPD), cystic fibrosis (e.g., cystic fibrosis wasting in adults), frailty, burns, infections (e.g., sepsis), muscular dystrophy, congestive heart failure, neurodegenerative conditions (e.g., Alzheimer's or pre-Alzheimer's syndrome, and/or amyotrophic lateral sclerosis), AIDS, protein malnutrition following long-term corticosteroid therapy, following non-union bone fracture, hip fracture, trauma, or major surgery (e.g., post-surgical problems), osteoporosis, long-term immobilization, cancer-related cachexia, sarcopenia (e.g., age-related sarcopenia), and gastrointestinal malabsorption (e.g., short bowel syndrome and/or Crohn's disease), e.g., in elderly subjects.

Examples of suitable conditions in which activity of GLP-1 and/or analogs thereof (e.g., GLP-1 receptor agonists) is beneficial include, without limitation, addiction (e.g., alcohol addiction and/or drug abuse), adipocyte dysfunction, Alzheimer's Disease, arthritis (e.g., osteoarthritis and/or gout), cardiovascular diseases and disorders (e.g., atherosclerosis (including coronary artery disease), angina pectoris, endothelial dysfunction, impaired vascular compliance, left ventricular hypertrophy, hypertension, myocardial infarction (e.g., necrosis and/or apoptosis), peripheral arterial disease, peripheral vascular disease, pulmonary hypertension, stroke (e.g., hemorrhagic stroke and/or ischemic stroke), thrombosis, transient ischemic attacks and/or congestive heart failure), cataract, cirrhosis, diabetes (including Type 1 diabetes and/or Type 2 diabetes; e.g., pre-diabetes (including impaired fasting plasma glucose), idiopathic Type 1 diabetes (Type 1b), latent autoimmune diabetes in adults, early-onset Type 2 diabetes, youth-onset atypical diabetes, malnutrition-related diabetes, gestational diabetes, and maturity onset diabetes of the young), diabetic neuropathy, diabetic nephropathy, diabetic retinopathy, dyslipidemia (including hyperlipidemia, hypertriglyceridemia, increased total cholesterol, high LDL cholesterol, and low HDL cholesterol), eating disorders (including binge eating syndrome, bulimia nervosa, excessive sugar craving, and syndromic obesity such as Prader-Willi and Bardet-Biedl syndromes), erectile dysfunction, fibrosis, gastrointestinal diseases and disorders (e.g., inflammatory bowel disease, short bowel syndrome, Crohn's disease, colitis, ulcerative colitis and/or irritable bowel syndrome), hepatocellular carcinoma, hyper-apo B lipoproteinemia, hyperglycemia, hyperinsulinemia, hyperuricemia, impaired cognition, impaired glucose metabolism, impaired glucose tolerance, insulin resistance (including hepatic insulin resistance), intermittent claudication, ketosis, kidney diseases and disorders (e.g., acute kidney disorder, chronic renal failure, glomerulosclerosis, tubular dysfunction, and/or pro-inflammatory changes to the proximal tubules), macular degeneration, metabolic acidosis, metabolic syndrome, non-alcoholic fatty liver, non-alcoholic steatohepatitis, obesity (including hypothalamic obesity and monogenic obesity) and related comorbidities (e.g., osteoarthritis and urine incontinence), osteoporosis, Parkinson's Disease, polycystic ovary syndrome, post-prandial lipemia, premenstrual syndrome, psoriasis, restenosis (e.g., vascular restenosis and/or restenosis after angioplasty), schizophrenia, skin and connective tissue diseases and disorders, sleep apnea, traumatic brain injury, ulcerations (e.g., foot ulcerations), visceral adipose deposition, and weight gain associated with a therapeutic agent (e.g., steroids and/or antipsychotics).

Examples of suitable conditions in which activity of GLP-2 and/or analogs thereof (e.g., GLP-2 receptor agonists) is beneficial include, without limitation, chemotherapy-induced adverse effects (e.g., abdominal cramping, bacterial translocation, diarrhea, gastrointestinal bleeding, malabsorption, mucositis and/or vomiting), cul-de-sac syndrome, digestion disorders, enteritis (e.g., radiation enteritis and/or infectious or post-infectious enteritis), inflammatory bowel disease, malabsorption syndromes, malnutrition (e.g., cachexia and/or anorexia), short-bowel syndrome (e.g., associated with surgical resection, congenital defect and/or disease-associated loss of absorption in the bowel), small intestine damage (e.g., associated with chemotherapy and/or toxic agents), celiac sprue (e.g., arising from gluten-induced enteropathy or celiac disease), tropical sprue, hypogammaglobulinemic sprue, ulcers (e.g., peptic ulcers, drug-induced ulcers, and/or ulcers associated with a pathogen), and ulcerative colitis.

A treatment according to any of the embodiments described herein (e.g., using GHRH, growth hormone, GLP-1, GLP-2, or related agent) may optionally be for improving a parameter associated with lipids and/or body structure. Examples of such improvements include, without limitation, decreasing cholesterol level, non-HDL cholesterol level, triglyceride level and/or total cholesterol/HDL cholesterol ratio, increasing lean body mass, and decreasing trunk fat, visceral fat, visceral adipose tissue, visceral adipose tissue/subcutaneous adipose tissue ratio and/or abdominal girth.

For conditions associated with chemotherapy, according to any of the respective embodiments described herein, the chemotherapeutic agent may be, for example, altretamine, bleomycin, busulfan, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, liposomal doxorubicin, leucovorin, lomustine, melphalan, mercaptopurine, mesna, methotrexate, mitomycin, mitoxantrone, oxaliplatin, paclitaxel, pemetrexed, pentostatin, procarbazine, raltitrexed, streptozocin, tegafur-uracil, temozolomide, thiotepa, thioguanine, topotecan, treosulfan, vinblastine, vincristine, vindesine, and/or vinorelbine.

Examples of suitable conditions in which activity (e.g., peripheral activity) of a kappa opioid receptor and/or kappa opioid receptor agonist is beneficial include, without limitation, congestive heart failure, edema (e.g., associated with congestive heart disease and/or inappropriate antidiuretic hormone secretion), glaucoma, hypertension, ileus (e.g., post-operative ileus and/or opioid-induced bowel dysfunction), inflammation (e.g., autoimmune inflammation, throat inflammation and/or inflammation associated with a drug, such as an NSAID), hypokalemia, hyponatremia, liver cirrhosis, nephrotic syndrome, pain (e.g., acute pain, chronic pain, cutaneous pain, hyperalgesia, neuropathic pain, ocular pain, somatic pain, throat pain, and/or visceral pain), pruritus, and tussis.

Examples of neuropathic pain treatable according to any of the respective embodiments of the invention include, without limitation, diabetic pain, chemotherapy-induced pain, migraine headache, nerve encroaching metastatic cancer pain, trigeminal neuralgia, viral pain (e.g., herpes zoster-associated pain), and neuropathic pain associated with traumatic injury and/or surgical procedures.

Examples of ocular pain treatable according to any of the respective embodiments of the invention include, without limitation, pain associated with chemical burn, conjunctivitis, corneal abrasion and/or irritation, corneal ulcer, episcleritis, herpes zoster ophthalmicus, interstitial keratitis, iritis (e.g., acute iritis), keratoconjunctivitis sicca, ocular laceration, orbital cellulites, orbital floor fracture, orbital pseudotumor, pemphigus, photo-refractive keratectomy, scleritis, sclerokeratitis, trachoma and/or uveitis.

Examples of throat pain treatable according to any of the respective embodiments of the invention include, without limitation, pain associated with bronchitis (e.g., acute bronchitis), burns (e.g., pharyngeal burns), cancer (e.g., laryngeal cancer), common cold, contact ulcers, gingivitis (e.g., acute necrotizing ulcerative gingivitis), herpes simplex lesions, influenza, mononucleosis, laryngitis (e.g., acute laryngitis), peritonsillar abscess, pharyngitis, rhinitis (e.g., allergic rhinitis), sinusitis (e.g., acute sinusitis), and/or tonsillitis.

Further examples of pain treatable according to any of the respective embodiments of the invention include, without limitation, pain associated with arthritis (e.g., rheumatoid arthritis and/or osteoarthritis), cancer (e.g., breakthrough cancer pain), cirrhosis, dysmenorrhea, endometriosis, a gastrointestinal disease or disorder (e.g., bile duct stone, cholecystitis, colitis, diverticulitis, dyspepsia, esophagitis, gallstone, gastritis, gastroenteritis, gastro-esophageal reflux disease, gastric or duodenal ulcer, inflammatory bowel disease, intestinal obstruction, irritable bowel syndrome, pancreatitis, perforated ulcer, ulcerative colitis), hepatic abscess, hepatitis, interstitial cystitis, kidney stone, menopause (e.g., hot flashes), ovarian cyst, mastitis, pelvic inflammatory disease, peritonitis, polynephritis (e.g., acute polynephritis), prostatitis, pyelonephritis (e.g., acute pyelonephritis), surgery (e.g., arthroplasty) or other medical procedure, such as a biopsy (e.g., following appendectomy, cervical biopsy, cholecystectomy colectomy, colonic resection, colonoscopy, colostomy, cystoscopy, endometrial biopsy, gastrectomy, hernia repair, hysterectomy, hysteroscopy, open colorectal surgery, pelvic laparoscopy, prostatectomy, splenectomy, tubal ligation, and/or vasectomy), a toxin (e.g., insect toxin), urinary tract stone, and/or uterine cramping.

Examples of pruritus treatable according to any of the respective embodiments of the invention include, without limitation, anal pruritus (e.g., associated with hemorrhoids), drug-induced pruritus (e.g., mu opioid-induced pruritus), ocular pruritus (e.g., associated with conjunctivitis), otitic pruritus, uremic pruritus (e.g., in subjects undergoing hemodialysis), and pruritus associated with cholestasis (e.g., associated with primary biliary cholangitis, intrahepatic cholestasis of pregnancy, chronic cholestatic liver disease, uremia, malignant cholestasis and/or jaundice), animal (e.g., insect) bites or stings and/or a dermatological disease or disorder (e.g., dermatitis (including atopic and contact dermatitis), lichen planus, lichen simplex chronicus, pediculosis, polycythemia vera, psoriasis, scabies, thyrotoxicosis, tinea pedis, urticarial and/or vaginitis).

In the context of medical conditions associated with bone fractures, the terms “treating” and “treatment” encompass, for example, substantially healing, at least in part, a bone fracture (e.g., a fracture non-union which does not heal without intervention), substantially increasing a rate at which a bone fracture heals, substantially ameliorating or preventing the appearance of symptoms of a bone fracture (e.g., pain, loss of functionality of a portion of the body, defective bone formation), and preventing or reducing the likelihood of a bone fracture occurring, for example, due to a medical condition (e.g., prophylaxis). Treatment of a bone fracture as described herein may optionally be performed in combination with standard treatments of bone fractures, such as immobilization of bones (e.g., with a cast) and/or surgery.

Examples of conditions associated with a bone fracture include, without limitation, a fracture non-union, any medical condition associated with a stress fracture (optionally the condition is a stress fracture per se),

Herein and in the art, the phrase “fracture non-union” refers to a medical condition in which a bone fracture is present, and there is no reasonable expectation that the fracture will heal without intervention.

The skilled person will be readily capable of determining a presence of a fracture non-union.

In some embodiments according to any of the embodiments relating to non-unions, a fracture non-union is determined based on non-consolidation at the fracture site 6 months after the fracture was formed, and/or based on an absence of progress in callus formation at the fracture site at 4 week intervals (e.g., as described by Giannotti et al. [Clin Cases Miner Bone Metab 2013, 10:116-120]).

Herein and in the art, the phrase “stress fracture” refers to a bone fracture caused by repeated stress over time (e.g., by running and/or jumping).

In some embodiments, treating a medical condition associated with a stress fracture comprises increasing a rate at which an existing stress fracture heals.

In some embodiments, treating a medical condition associated with a stress fracture comprises reducing the likelihood of a stress fracture occurring, for example, in a subject susceptible to stress fractures. Examples of subjects susceptible to stress fractures include, without limitation, athletes, runners, soldiers and other people subject to considerable physical exercise.

Herein, the phrase “bone defect” encompasses any missing portion of a bone, including, bone missing due to trauma (e.g., wherein a bone fracture results in a missing bone fragment), surgery (e.g., wherein bone is surgically removed in order to remove cancer cells), resorption of bone, an acquired medical condition (e.g., wherein an acquired medical condition causes a portion of a bone to disappear via resorption) and/or congenital conditions (e.g., wherein a congenitally misshapen bone is associated with one or more defects in the bone structure), a space between a bone and an implant intended to be osseointegrated with the bone (including, but not limited to, an implant anchored in a bone, for example, via a bolt or screw).

Examples of medical conditions involving resorption of bone include, without limitation, bone resorption associated with inflammatory conditions (e.g., periodontitis), which may comprise resorption of bone near the site of inflammation, and resorption of alveolar bone associated with a missing tooth.

Herein, the terms “osseointegration” and “osseointegrated” refer to formation of a direct structural connection (e.g., without intervening connective tissue) between living bone and an implant; and includes, but is not limited to, growth of bone into an implant (e.g., a porous implant), a process also known in the art as “osseoincorporation”.

In the context of medical conditions associated with bone defects, the terms “treating” and “treatment” encompass, for example, substantially healing, at least in part, a bone defect (e.g., replacement of at least a portion of missing bone by bone regeneration), substantially increasing a rate at which a bone defect heals (e.g., a rate of bone regeneration), substantially ameliorating or preventing the appearance of symptoms of a bone defect (e.g., pain, loss of functionality of a portion of the body, defective bone formation), and preventing or reducing formation of a bone defect (e.g., prophylaxis), for example, formation of a bone defect by bone resorption. Treatment of a bone defect as described herein may optionally be performed in combination with standard treatments of the respective bone defect.

In some embodiments according to any of the respective embodiments described herein, the medical condition is resorption of alveolar bone. In some of these embodiments, the method or treatment is for preserving and/or regenerating alveolar bone. Examples of resorption of alveolar bone include, without limitation, resorption associated with a missing tooth and resorption associated with inflammation (e.g., periodontitis).

In some embodiments, the method or treatment is for preserving and/or regenerating alveolar bone surrounding a dental implant (e.g., a dental implant which comprises or supports a prosthetic tooth, crown, dental bridge and/or fixed denture), for example, to hold the dental implant in place, thereby increasing the utility of the implant and/or the likelihood of success of the dental implantation. In some embodiments, the method or treatment is effected following implantation of a dental implant, for example, in order to promote regeneration of alveolar bone (e.g., alveolar bone characterized by a bone defect associated with resorption of the bone due to a missing tooth and/or periodontitis). In alternative or additional embodiments, the method or treatment is effected prior to implantation of a dental implant, for example, in order to preserve alveolar bone by preventing or reducing alveolar bone resorption (e.g., upon loss of a tooth, when a significant amount of time is expected to pass before implantation of a dental implant.

In some embodiments according to any of the respective embodiments described herein, the bone defect is in the skull (cranium or lower jawbone). In some embodiments, the bone defect is a calvarial bone defect.

Without being bound by any particular theory, it is believed that bone in the skull (e.g., in the calvaria) is particularly susceptible to poor healing of bone defects, in which promotion of bone growth would be advantageous.

In some embodiments according to any of the respective embodiments described herein, the method and/or treatment comprises promoting osseointegration of an implant, for example, by promoting bone growth in a space between a bone (e.g., calvarial bone) and the implant. The medical condition may optionally be any medical condition for which osseointegration of an implant is beneficial.

Examples of implants for which osseointegration may be promoted include, without limitation, dental implants, bone grafts (e.g., bone allografts), chin implants, craniofacial prostheses (e.g., artificial ears, eyes and/or noses), bone-anchored limb prostheses, bone-anchored hearing aids, and joint prostheses (e.g., for hip and/or knee replacement).

Herein, the term “implant” refers to any device, wherein at least a portion of the device is placed in a subject, and encompasses man-made devices and transplanted tissue, and may comprise synthetic materials, an autograft (e.g., bone harvested from a different region of the subject, such as the iliac crest or chin), an allograft (e.g., bone harvested from an individual other than the subject, optionally a cadaver), a xenograft (e.g., bone from a different species, optionally bovine bone or coral) or any combination thereof. Examples of synthetic material which may be included in an implant (e.g., an implant intended to be osseointegrated) include, without limitation, hydroxyapatite, calcium carbonate, tricalcium phosphate, polymers (e.g., poly(methyl methacrylate), poly(hydroxyethyl methacrylate)), ceramics and metals (e.g., titanium).

In some embodiments of any one of the embodiments described herein relating to treatment of osteoporosis and/or a condition associated with a bone fracture or bone defect using PTH, oral administration according to any of the respective embodiments described herein is effected from 1 to 4 times per day. In some such embodiments, the oral administration according to any of the respective embodiments described herein is effected from 1 to 3 times per day. In some embodiments, the oral administration according to any of the respective embodiments described herein is effected once or twice per day. In some embodiments, the oral administration according to any of the respective embodiments described herein is effected once per day.

In some embodiments of any one of the embodiments described herein relating to treatment of a condition associated with a bone fracture or bone defect using PTH is effected once per day or less. In some such embodiments, the oral administration is effected once every two days. In some such embodiments, the oral administration is effected twice per week. In some such embodiments, the oral administration is effected once per week or less.

In some embodiments of any one of the embodiments described herein relating to oral administration once per day or less frequently, the treatment is a prophylactic treatment (for preventing or reducing the likelihood and/or size of a bone fracture and/or bone defect), that is, the subject does not necessarily have a bone fracture and/or bone defect at the time of treatment.

In some embodiments, the prophylactic treatment is for stress fractures, for example, in a subject susceptible to stress fractures (e.g., as described herein).

In some embodiments, the prophylactic treatment is for preventing or reducing an alveolar bone defect associated with resorption of alveolar bone, for example, in a subject susceptible to alveolar bone resorption (e.g., as described herein). Subjects afflicted by periodontitis and/or subjects missing a tooth are non-limiting examples of subjects susceptible to alveolar bone resorption.

Without being bound by any particular theory, it is believed that relatively low dosages (e.g., as effected by a relatively low frequency of oral administration) are more suitable than high dosages for prophylactic applications.

Compositions for oral administration (as described herein) may be particularly convenient for indications in which frequent administration of a therapeutically active agent is desirable.

In some embodiments of any one of the embodiments described herein relating to treatment of hypoparathyroidism with PTH, the oral administration according to any of the respective embodiments described herein is effected at least twice per day (e.g., from 2 to 6 times per day). In some such embodiments, the oral administration according to any of the respective embodiments described herein is effected at least 3 times per day (e.g., from 3 to 6 times per day). In some embodiments, the oral administration according to any of the respective embodiments described herein is effected at least 4 times per day (e.g., from 4 to 6 times per day).

Without being bound by any particular theory, it is believed that frequent administration of PTH as described in any of the respective embodiments herein provides a relatively steady increase in PTH levels in the body, which is advantageous in the treatment of hypoparathyroidism.

Without being bound by any particular theory, it is believed that compositions according to some embodiments of the invention are characterized by rapid absorption (e.g., associated with absorption via stomach tissue, prior to reaching the intestines), which is particularly advantageous for certain applications; for example, for obtaining a pulse-like pharmacokinetic profile (e.g., of GHRH or PTH), or for treatment of acute, difficult-to-tolerate conditions or symptoms, such as pain and/or pruritus.

In some embodiments, the rapid administration is characterized by an average Tmax of the composition upon oral administration is no more than 60 minutes, or no more than 50 minutes, or no more than 40 minutes, or no more than 30 minutes, or no more than 20 minutes, or no more than 15 minutes, or even no more than 10 minutes.

As used herein the term “Tmax” refers to the time from administration to the time point at which maximal concentration of an administered agent in the blood (e.g., plasma levels) occurs, and can be determined by measuring levels of the agent at various time points following administration, as exemplified herein.

In some embodiments, the composition (e.g., composition unit dosage form) is formulated such that absorption of the therapeutically active agent following oral administration of the composition is characterized by a ratio of AUC to Cmax which is 3 hours or lower. In some embodiments, the ratio of AUC to Cmax is 2 hours or lower. In some embodiments, the ratio of AUC to Cmax is 90 minutes or lower. In some embodiments, the ratio of AUC to Cmax is 60 minutes or lower. In some embodiments, the ratio of AUC to Cmax is 50 minutes or lower. In some embodiments, the ratio of AUC to Cmax is 40 minutes or lower. In some embodiments, the ratio of AUC to Cmax is 30 minutes or lower. In some embodiments, the ratio of AUC to Cmax is 20 minutes or lower. In some embodiments, the ratio of AUC to Cmax is 15 minutes or lower. In some embodiments, the ratio of AUC to Cmax is 10 minutes or lower.

As used herein the term “AUC” refers to the area under a curve which represents levels of an administered agent in the blood (e.g., plasma levels) as a function of time following administration, and can be determined by measuring plasma levels of the agent at various time points following administration, as exemplified herein.

As used herein the term “Cmax” refers to the maximal concentration of an administered agent in the blood (e.g., plasma levels), and can be determined by measuring levels of the agent at various time points following administration, as exemplified herein.

The ratio of AUC to Cmax (i.e., AUC divided by Cmax) will depend on the nature of the pharmacokinetic profile of the composition, particularly on the shape of the curve which represents levels of the therapeutically active agent in the blood (e.g., plasma levels) as a function of time following administration. Pharmacokinetic profiles characterized by a sharp increase and decrease within a brief period of time will tend to have a relatively low ratio of AUC to Cmax, whereas pharmacokinetic profiles characterized by a more gradual increase and decrease over a broader period of time will tend to have a relatively high ratio of AUC to Cmax.

Thus, without being bound by any particular theory, it is believed that a ratio of AUC to Cmax which is 3 hours or lower, as described herein according to any of the respective embodiments, is associated with a relatively sharp increase and decrease of levels of therapeutically active agent in the blood.

The ratio of AUC to Cmax is optionally calculated based on data from multiple administrations of the composition. In such cases, a ratio of AUC to Cmax is preferably calculated for each administration, and then the ratios calculated for each administration may be averaged.

Similarly, the Cmax and/or Tmax are optionally calculated based on data from multiple administrations of the composition. In such cases, a Cmax and/or Tmax value is preferably calculated for each administration, and then the Cmax and/or Tmax values calculated for each administration may be averaged.

Without being bound by any particular theory, it is believed that averaging data (e.g., measured blood levels of therapeutically active agent) from different administrations of therapeutically active agent will frequently result in a broader curve, a lower Cmax value, and larger ratio of AUC to Cmax, than that which is observed after a single administration. Hence, a Cmax value and a ratio of AUC to Cmax calculated for averaged data (as opposed to an average of ratios calculated for each administration, as described hereinabove) is a less accurate indicator of the effect of the composition following administration.

### Formulation of Compositions:

Each of the compositions and unit dosage forms described herein optionally consist essentially of the functional ingredients described hereinabove (e.g., a therapeutically active agent, an absorption enhancer, an alkaline group-containing polymer and a non-polymeric proton-accepting agent, each according to any of the embodiments described herein in any of the respective sections herein), or alternatively, the composition further comprises suitable pharmaceutically acceptable carriers and/or excipients.

Hereinafter, the phrases “physiologically acceptable carrier” and “pharmaceutically acceptable carrier”, which may be interchangeably used, refer to a carrier or a diluent that does not cause significant irritation to an organism and does not abrogate the activity (e.g., biological activity) and properties of the functional ingredient (e.g., a therapeutically active agent). An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch (e.g., types of non-modified starch), cellulose derivatives, gelatin, vegetable oils and polyethylene glycols.

In some embodiments of any one of the embodiments described herein, the composition is formulated as a solid composition, e.g., a solid unit dosage form. In some embodiments, the composition is formulated as a tablet or as a combination of tablets (e.g., a plurality of minitablets). In some embodiments, the composition is formulated as a capsule, as described herein, which comprises, for example, a plurality of minitablets and/or a powder, and which has a coating that is dissolvable in a gastric fluid (e.g., in gastric pH) or otherwise releases the capsule's content immediately (e.g., within less than 5 minutes, or less than 2 minutes or less than 1 minutes) upon contacting aa gastric fluid.

Ingredients of the formulation (including, but not limited to, any two or more of the alkaline group-containing polymer, absorption enhancer, the non-polymeric proton-accepting agent and therapeutically active agent of any of the respective embodiments described herein may optionally be mixed in a homogeneous manner or be distributed throughout the composition in a heterogeneous manner.

Techniques for formulation and administration of drugs may be found in “Remington's Pharmaceutical Sciences,” Mack Publishing Co., Easton, PA, latest edition, which is incorporated herein by reference.

In some embodiments of any one of the embodiments described therein, the pharmaceutical compositions can be formulated to contain only the alkaline group-containing polymer, absorption enhancer and therapeutically active agent. This pharmaceutical composition can thereafter be co-administered with at least one proton-accepting agent to a subject in need thereof. In some embodiments, the proton-accepting agent may be administered concurrently with the administration of the pharmaceutical composition. In some embodiments, the proton-accepting agent may be administered within 5 minutes, 10 minutes, 15 minutes, 20 minutes, 25 minutes, 30 minutes, 35 minutes, 40 minutes, 45 minutes, 50 minutes, 55 minutes, 60 minutes, 120 minutes, 180 minutes, or 240 minutes, before or after the administration of the pharmaceutical composition.

Pharmaceutical compositions and unit dosage forms of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions and unit dosage forms for use in accordance with some embodiments of the invention may thus be formulated in conventional manner using one or more physiologically acceptable carriers comprising excipients and auxiliaries, which facilitate processing of the active ingredients into preparations which, can be used pharmaceutically.

The pharmaceutical composition and unit dosage forms can be formulated readily by combining the active compounds with pharmaceutically acceptable carriers well known in the art as being suitable for oral administration. Such carriers optionally facilitate formulation of the pharmaceutical composition as tablets (including minitablets), pellets, pills, dragees, capsules, powders, granules, elixirs, tinctures, liquids, gels, syrups, slurries, suspensions, emulsions and the like, for oral administration to a patient. Pharmacological preparations for oral use can be made using a solid excipient, optionally grinding the resulting mixture, and processing the mixture of granules, after adding suitable auxiliaries if desired, to obtain tablets or dragee cores. Pharmaceutical composition and unit dosage forms suitable for oral administration include, but are not limited to, rapid-release, time controlled-release, extended-release, and delayed-release pharmaceutical dosage forms. In some of any of the respective embodiments, a pharmaceutical composition formulated for oral administration is a solid composition, for example, tablet, capsule, powder or granules.

Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose; and/or physiologically acceptable polymers such as polyvinylpyrrolidone (PVP). If desired, lubricants may be added, such as talc or magnesium stearate.

In some embodiments of any one of the embodiments described herein, any one of the compositions or unit dosage forms described herein (e.g., formulated as a tablet) further comprises a lubricant. In some embodiments, the lubricant is included in a concentration of 5 weight percent or less, optionally 2 weight percent or less, and optionally about 1 weight percent. In some embodiments, the composition or unit dosage form described herein (e.g., formulated as a tablet) consists essentially of the therapeutically active agent (as described herein), absorption enhancer, lubricant, alkaline group-containing polymer, and optional protease inhibitor (as described herein). In some embodiments, the lubricant is magnesium stearate.

The compositions or unit dosage forms described herein (e.g., formulated as a tablet) may alternatively or in addition comprise additives and/or additional agents, such as, for example, antioxidants, glidants, solvents, odor absorbers, chelating agents, preservatives, thickeners, colorants or coloring agents (pigments, nacres, water-soluble dyestuffs), dispersants, fillers, flavoring agents and bactericides.

Dragee cores are optionally provided with suitable coatings. For this purpose, concentrated sugar solutions may be used which may optionally contain gum arabic, talc, polyvinylpyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol, pullulan, or HPMC. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added.

In some of any of the embodiments described herein, the pharmaceutical composition and/or unit dosage form according to any of the respective embodiments described herein is devoid of a gastroenteric coating, or otherwise comprises a coating that is dissolvable in the GI tract (e.g., in gastric fluid) and/or which otherwise releases the contents immediately (e.g., within no more than 5 minutes, or no more than 2 minutes or no more than one minute) upon contacting a gastric fluid.

Pharmaceutical compositions suitable for use in context of some embodiments of the invention include compositions wherein the therapeutically active agent is contained in an amount effective to achieve the intended purpose. More specifically, the composition preferably comprises a therapeutically effective amount of therapeutically active agent, that is, an amount of therapeutically active agent effective to prevent, alleviate or ameliorate symptoms of a disorder or prolong the survival of the subject being treated. Furthermore, an amount of absorption enhancer is preferably effective for enhancing absorption of the therapeutically active agent (e.g., in a manner described herein); and an amount of protease inhibitor (if present) is preferably effective for inhibiting degradation of the therapeutically active agent (e.g., a polypeptide agent) by a protease.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the therapeutically active agent described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation and dosage can be chosen by the individual physician in view of the patient's condition. (See e.g., Fingl et al., 1975, in “The Pharmacological Basis of Therapeutics”, Ch. 1 p.1).

Dosage amount and interval may be adjusted individually to provide levels (e.g., plasma levels) of the therapeutically active agent sufficient to induce or suppress a biological effect (minimal effective concentration, MEC). The MEC will vary for each preparation, but can be estimated from in vitro data. Dosages necessary to achieve the MEC will depend on individual characteristics. Detection assays can be used to determine plasma concentrations.

Depending on the severity and responsiveness of the condition to be treated, dosing can be of a single or a plurality of administrations, with course of treatment lasting from several hours to several weeks or until cure is effected or diminution of the disease state is achieved.

The amount of a composition to be administered will, of course, be dependent on the subject being treated, the severity of the affliction, the manner of administration, the judgment of the prescribing physician, etc.

Each dosing may optionally be effected using a single dosage form, or a plurality of unit dosage forms. A plurality of unit dosage forms may optionally be used merely for convenience (e.g., wherein two or three unit dosage forms are used), or alternatively, to reduce variability in absorption according to any of the respective embodiments described in International Patent Application Publication No. WO 2018/033927, for example, wherein at least three or at least four unit dosage forms are used (e.g., from 3 to 10 or from 4 to 10, including any intermediate values and subranges therebetween).

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention may also be prepared (e.g., as described herein), placed in an appropriate container, and labeled for treatment of an indicated condition, as is further detailed herein.

According to exemplary embodiments, the composition is a formulated as a solid form, for example, as a solid unit dosage form such as a tablet or a mini-tablet.

According to exemplary embodiments, an amount of the therapeutically active agent in a solid unit dosage form ranges from 0.1 to 5 mg, or from 0.1 to 2 mg, or from 0.1 to 1 mg, or from 0.2 to 3 mg, or from 0.2 to 2 mg, or from 0.2 to 1 mg, including any intermediate values and subranges therebetween. Higher amounts, for example, up to 100 mg are also contemplated, in accordance with any of the embodiments described herein.

### Additional Definitions

Herein, the term “polypeptide” refers to a polymer comprising at least 4 amino acid residues linked by peptide bonds or analogs thereof (as described herein), and optionally only by peptide bonds per se. In some embodiments, the polypeptide comprises at least 10 amino acid residues or analogs thereof. In some embodiments, the polypeptide comprises at least 20 amino acid residues or analogs thereof. In some embodiments, the polypeptide comprises at least 30 amino acid residues or analogs thereof. In some embodiments, the polypeptide comprises at least 50 amino acid residues or analogs thereof. The term “polypeptide” encompasses native polypeptides (e.g., degradation products, synthetically synthesized polypeptides and/or recombinant polypeptides), including, without limitation, native proteins, fragments and substituted derivatives of native proteins and homologs of native proteins and/or fragments and/or substituted derivatives thereof; as well as peptidomimetics (typically, synthetically synthesized polypeptides) and peptoids and semipeptoids which are polypeptide analogs, which may have, for example, modifications rendering the polypeptides more stable while in a body or more capable of penetrating into cells. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided herein.

Peptide bonds (—CO—NH—) within the polypeptide may be substituted, for example, by N-methylated amide bonds (—N(CH₃)—CO—), ester bonds (—C(═O)—O—), ketomethylene bonds (—CO—CH₂—), sulfinylmethylene bonds (—S(═O)—CH₂—), α-aza bonds (—NH—N(R)—CO—), wherein R is any alkyl (e.g., methyl), amine bonds (—CH₂—NH—), sulfide bonds (—CH₂—S—), ethylene bonds (—CH₂—CH₂—), hydroxyethylene bonds (—CH(OH)—CH₂—), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention (e.g., a therapeutically active agent described herein) may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables A and B below list naturally occurring amino acids (Table A), and non-conventional or modified amino acids (e.g., synthetic, Table B) which can be used with some embodiments of the invention.

TABLE A

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE B

| Non-conventional amino | | Non-conventional amino | |
|---|---|---|---|
| acid | Code | acid | Code |
| ornithine | Orn | hydroxyproline | Hyp |
| α-aminobutyric acid | Abu | aminonorbornyl- | Norb |
| | | carboxylate | |
| D-alanine | Dala | aminocyclopropane- | Cpro |
| | | carboxylate | |
| D-arginine | Darg | N-(3- | Narg |
| | | guanidinopropyl)glycine | |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl- | Nmhphe |
| | | homophenylalanine | |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α- | Nmaabu |
| | | methylbutyrate | |
| α-aminoisobutyric acid | Aib | N-methyl-α- | Nmaib |
| | | aminoisobutyrate | |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl- | Mhphe |
| | | homophenylalanine | |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2- | Nbhm | N-(N-(2,2-diphenylethyl) | Nnbhm |
| diphenylethyl)glycine | | carbamylmethyl-glycine | |
| N-(3,3- | Nbhe | N-(N-(3,3-diphenylpropyl) | Nnbhe |
| diphenylpropyl)glycine | | carbamylmethyl-glycine | |
| 1-carboxy-1-(2,2-diphenyl | Nmbc | 1,2,3,4- | Tic |
| ethylamino)cyclopropane | | tetrahydroisoquinoline-3- | |
| | | carboxylic acid | |
| phosphoserine | pSer | phosphothreonine | pThr |
| phosphotyrosine | pTyr | O-methyl-tyrosine | |
| 2-aminoadipic acid | | hydroxylysine | |

The polypeptides of some embodiments of the invention (e.g., a therapeutically active agent described herein) are preferably utilized in a linear form, although it will be appreciated that in cases where cyclization does not severely interfere with polypeptide characteristics, cyclic forms of the polypeptide can also be utilized.

In some embodiments of any one of the embodiments described herein, the polypeptide is water-soluble, as defined herein.

Water-soluble polypeptides preferably include one or more non-natural or natural polar amino acids, including but not limited to serine and threonine which are capable of increasing polypeptide water-solubility due to their hydroxyl-containing side chain. Optionally, a homolog of a polypeptide is selected so as to be more water-soluble than the parent polypeptide, for example, by replacing one or more amino acids in the polypeptide with polar amino acids.

The polypeptides of some embodiments of the invention (e.g., a therapeutically active agent described herein) may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing polypeptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the polypeptide compounds of some embodiments of the invention (e.g., a therapeutically active agent described herein) involves solid phase peptide synthesis.

Large scale polypeptide synthesis is described by Andersson et al. [Biopolymers 2000; 55:227-250].

The term “nucleic acid” refers to a single stranded or double stranded oligomer or polymer of monomeric units of ribonucleic acid (RNA) or deoxyribonucleic acid (DNA) or mimetics thereof. This term includes nucleic acids composed of naturally-occurring bases, sugars and covalent internucleoside linkages (e.g., backbone) as well as nucleic acids having non-naturally-occurring portions which function similarly to respective naturally-occurring portions.

Nucleic acids designed according to the teachings of some embodiments of the invention can be generated according to any nucleic acid synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the nucleic acids is well within the capabilities of one skilled in the art and can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The nucleic acid of some embodiments of the invention is of at least 8, at least 13, at least 17, at least 18, at least 19, at least 20, at least 22, at least 25, at least 30 or at least 40, bases specifically hybridizable with sequence alterations described hereinabove.

The nucleic acid of some embodiments of the invention is no more than 200, or no more than 50 (e.g., from 8 to 50), or no more than 25 (e.g., from 13 to 25) bases in length.

The nucleic acids of some embodiments of the invention may comprise heterocyclic nucleosides consisting of purines and the pyrimidines bases, bonded in a 3′ to 5′ phosphodiester linkage.

Preferably used nucleic acids are those modified in either backbone, internucleoside linkages or bases, as is broadly described hereunder.

Specific examples of preferred nucleic acids useful according to some embodiments of the invention include nucleic acids containing modified backbones or non-natural internucleoside linkages. Nucleic acids having modified backbones include those that retain a phosphorus atom in the backbone, as disclosed in U.S. Pat. Nos. 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified nucleic acid backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified nucleic acid backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other nucleic acids which can be used according to some embodiments of the invention, are those modified in both sugar and the internucleoside linkage, i.e., the backbone, of the nucleotide units are replaced with novel groups. The base units are maintained for complementation with the appropriate polynucleotide target. An example for such a nucleic acid mimetic, includes peptide nucleic acid (PNA). A PNA refers to a nucleic acid where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in some embodiments of the invention are disclosed in U.S. Pat. No. 6,303,374.

Nucleic acids of some embodiments of the invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds according to some embodiments of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi Y S et al. (1993) Antisense Research and Applications, CRC Press, Boca Raton 276-278] and are presently preferred base substitutions, even more particularly when combined with 2′-O-methoxyethyl sugar modifications. Still further base substitutions include the non-standard bases disclosed in U.S. Pat. Nos. 8,586,303, 8,614,072, 8,871,469 and 9,062,336, all to Benner et al: for example, the non-standard dZ:dP nucleobase pair which Benner et al has shown can be incorporated into DNA by DNA polymerases to yield amplicons with multiple non-standard nucleotides.

Herein, a “homolog” of a given polypeptide or nucleic acid refers to a polypeptide or nucleic acid that exhibits at least 80% homology, preferably at least 90% homology, and more preferably at least 95% homology, and more preferably at least 98% homology to the given polypeptide or nucleic acid. In some embodiments, a homolog of a given polypeptide/nucleic acid further shares a therapeutic activity with the given polypeptide/nucleic acid. The percentage of homology refers to the percentage of amino acid residues in a first polypeptide sequence which match a corresponding residue of a second polypeptide sequence to which the first polypeptide is being compared, or the percentage of nucleotides a first nucleic acid sequence which match a corresponding nucleotide of a second nucleic acid sequence to which the first nucleic acid is being compared. Generally, the polypeptides/nucleic acids are aligned to give maximum homology. A variety of strategies are known in the art for performing comparisons of amino acid or nucleotide sequences in order to assess degrees of identity, including, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2:482); the homology alignment algorithm of Needleman and Wunsch (J. Mol. Biol., 1970, 48:443); the search for similarity method of Pearson and Lipman (Proc. Natl. Acad. Sci. (USA), 1988, 85:2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW etc. When utilizing BLAST and Gapped BLAST programs, default parameters of the respective programs may be used. Alternatively, the practitioner may use non-default parameters depending on his or her experimental and/or other requirements (see for example, the Web site having URL www(dot)ncbi(dot)nlm(dot)nih(dot)gov).

Herein, the terms “amine” and “amino” each refer to a —NR′R″ group, wherein R′ and R″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Alternatively, R′ and R″ may optionally be linked to form a heteroalicyclic ring (as defined herein). Optionally, R′ and R″ and R′″ are hydrogen or alkyl comprising 1 to 4 carbon atoms. Optionally, at least one of R′ and R″ is hydrogen and optionally both are hydrogen. When substituted, the carbon atom of an R′ and R″ hydrocarbon moiety which is bound to the nitrogen atom of the amine is not substituted by oxo, such that R′ and R″ are not (for example) carbonyl, C-carboxy or amide, as these groups are defined herein.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

An “azide” group refers to a —N═N⁺═N⁻ group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O) OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S) NR″-group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′″ are as defined herein.

As used herein, the term “thiohydrazide” describes a —C(═S)—NR′—NR″R′″ group, where R′, R″ and R″ are as defined herein.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(=NRd)- group, where Ra, Rb and Rd are as defined herein.

For any of the embodiments described herein, each of the compounds described herein (including therapeutically active agents, absorption enhancers, and acid-neutralizing polymeric and non-polymeric substances) may be in a form of a salt, for example, a pharmaceutically acceptable salt, and/or in a form of a prodrug, as long as its intended use (e.g., acid-neutralization) is maintained.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein (including therapeutically active agents, absorption enhancers and polymers), including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an(S) configuration and any combination, and compounds according to some embodiments of the present invention, can have any their chiral centers exhibit an (R) or an(S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is expected that during the life of a patent maturing from this application many relevant therapeutically active agents and many relevant treatments of conditions by therapeutically active agents will be developed, and the scope of the phrases “therapeutically active agent” and “condition treatable by . . . therapeutically active agent” are intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

Croscarmellose sodium (Parteck® CCS; crosslinked; CAS No. 74811-65-7) was obtained from Merck.

Sodium alginate (CAS No. 9005-38-3), average MW 300-350 kDa, viscosity at room temperature 350-500 mPa (non-crosslinked), was obtained from Carl Roth.

Sodium carboxymethylcellulose (CMC), CAS No. 9004-32-4 (not cross linked), low viscosity (43 mPa at room temperature) was obtained from Merck or Ashland.

Sodium starch glycolate Type A (Primojel®; cross-linked; CAS No. 9063-38-1), average MW 2,000 kDa, viscosity at room temperature lower than 200 mPa, was obtained from DFE Pharma.

Histidine (CAS No. 36653-82-4) obtained from Sigma Aldrich.

L-Arginine (CAS No 74-79-3) was obtained from Tokyo Chemical Industry ltd.

Potassium Carbonate (CAS No. 584-08-7) was obtained from Sigma Aldrich.

Sodium Carbonate (CAS No. 497-19-8) was obtained from Riedel-de-Haen.

Sodium Decanoate (CAS No. 1002-62-6) was obtained from Algene.

Sodium Octanoate (CAS No. 1984 Jun. 1) was obtained from Sigma Aldrich.

SNAC (CAS No 203787-91-1) was synthesized in-house.

### pH Determination Using pH Meter:

Test substances were dissolved in various volumes of 0.1 M HCl solution (pH 1.2) at concentrations of 1, 2, 4, 10, 20, 50, 100 and 150 mg/ml. The solution pH was determined using an MP-103 pH-meter (MRC, Israel) equipped with ELC-10-00 electrode (MRC, Israel) or with a thin electrode HI1083 (HANNA instruments Inc.), shortly after dissolution and up to 26 hours later in order to ensure that the pH remained stable. The highest pH value obtained during the experimental time period was recorded in order to compare the acid-neutralizing capacity of each substance.

Other pH measurements were performed by adding a suitable colorimetric pH indicator solution to the tested medium.

### SNAC Solubility Determination in Gastric Conditions:

Round tablets of 7-millimeter diameter comprising a fixed amount of SNAC (80 mg) and SSG (20 mg) were prepared on a manual dental press at about a 1-ton compression force for 10 seconds. All tablets constituted the same amount of SNAC, SSG (an acid-neutralizing polymer) and various small (non-polymeric) proton-accepting agents at a quantity of 1:1, 0.75:1, 0.5:1, or 0.25:1 mol ratio to SNAC.

In order to test the ability of the tested formulations to protect SNAC, tablets were dissolved in a biorelevant medium, simulating the gastric fluid. Tests were therefore performed in HCl solution featuring pH of about 1.6 (+/−0.1), representing an average pH of a fasted stomach (which is reported to be in the range of 1.2-2). Six tablets of each of the tested formulations were submerged in a plastic tube (each tablet in a separate tube) containing 10 ml of the HCl solution pre-heated to 37° C., and were subjected to a constant rotary mixing (30 rpm) inside an oven set to 37° C. A small volume of 10 ml was selected in order to mimic the physiological conditions inside a fasted stomach, where the tablet is locally exposed to the surrounding medium and not to the entire liquid content. The active soluble fraction of SNAC was evaluated at six time points: 2, 5, 10, 15, 20 and 30 minutes. At each time point, one of the six tested tubes in each of tested sets of formulations was removed from the oven, and a 2 ml-sample was withdrawn therefrom, filtered through a PTFE 0.22 micrometer filter, and diluted 15-fold in mobile phase (45:55 ACN: 0.1% v/v H3PO4 85% in water). The amount of soluble SNAC in each sample, which represents the material available to act as permeability enhancer, was determined by High-Performance Liquid Chromatography (HPLC).

HPLC analysis was performed on Agilent 1100 series HPLC equipped with UV detector, Cosmosil C18 250*4.6 mm, 5 μm column, using 45:55 ACN: 0.1% v/v H3PO4 85% in water as eluent.

### Example 1

### Effect of Basic Polymers on pH in Simulated Gastric Fluid

The effects of basic polymers on gastric fluid and on the behavior of SNAC in gastric fluid was investigated using HCl solutions as simulated gastric fluid.

SNAC and the basic polymers sodium alginate, sodium carboxymethylcellulose in non-crosslinked (Na-CMC) and crosslinked (croscarmellose sodium, CCS) forms, and sodium starch glycolate (SSG) were dissolved in various volumes of 0.1 M HCl solution (pH 1.2) at concentrations of 1, 2, 4, 10, 20, 50, 100 and 150 mg/ml, and the solution pH was determined using an MP-103 pH-meter (MRC, Israel) equipped with ELC-10-00 electrode (MRC, Israel), as described in the Materials and Methods section hereinabove.

As shown in FIG. 1, upon addition to a HCl solution (pH 1.2), SNAC dissolved rapidly and elevated the pH in a concentration-dependent manner, wherein the pH 5 minutes after dissolution was about 1.5 upon addition of 10 mg/ml SNAC, 6.5 upon addition of 20 mg/ml SNAC, and about 7.5 upon addition of 100 mg/ml SNAC.

These results indicate that a substantial proportion of SNAC is converted under gastric conditions from a salt to the corresponding carboxylic acid (NAC), which is substantially less water soluble than the salt and which is not an effective permeation enhancer.

The effect of pH on peptide proteolysis by pepsin was determined by incubating 13.5 μg/ml human parathyroid hormone (1-34) (hPTH (1-34)) with 150 μg/ml pepsin, at various pH values, and determining the percentage of hPTH (1-34) which remained. The amount of hPTH (1-34) remaining upon addition to 150 μg/ml pepsin at 37° C. was determined in media with different pH values, followed by immediate (10 second) vortex and centrifugation for 3 minutes at 4° C., 4000 RCF. The following media were used: simulated gastric fluid (pH 2), prepared from 0.01 M HCl and NaCl; phthalate buffer (pH 4 or 5); and phosphate buffer (pH 6 or 7).

As shown in FIG. 2, proteolysis of hPTH (1-34) was inhibited when the pH increased to about pH 6 or higher.

Taken together, the above results indicate that SNAC can inhibit peptide degradation by pepsin under gastric conditions by raising the pH, at the cost of loss of at least a portion of the active salt form of the absorption enhancer.

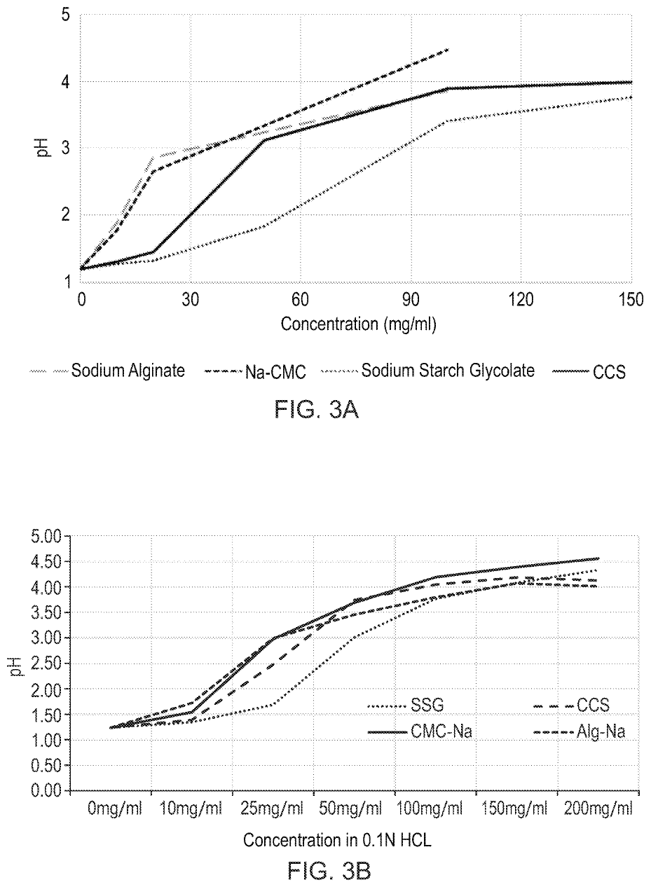

In order to obtain a beneficial rise in pH with a reduced loss of the active form of the absorption enhancer, the effect of various concentrations of different polymers containing carboxylate groups (sodium alginate, sodium starch glycolate, sodium carboxymethylcellulose, and croscarmellose sodium) on pH upon addition to a 0.1 M HCl solution (pH 1.2) was also evaluated. FIG. 3A presents pH values determined using an MP-103 pH-meter (MRC, Israel) equipped with ELC-10-00 electrode (MRC, Israel) and FIG. 3B presents pH values determined using an MP-103 pH-meter (MRC, Israel) equipped with a thin electrode HI1083 (HANNA instruments Inc.).

As shown in FIGS. 3A and 3B, each of the tested polymers exhibited a concentration-dependent increase in pH, with pH values of about 4-4.5 being obtained at the higher concentrations tested.

The sodium alginate, Na-CMC, CCS and sodium starch glycolate tested powders form gels (of various viscosities) upon addition to aqueous media, and the dispersion of the polymers in such gels may be inhomogeneous. In order to assess whether the acid-neutralizing capacity of the polymer is determined by the concentration of the polymer in a gel, preliminary studies were performed with polymers in a powder form inserted to the acidic medium without mixing. pH values were determined using a suitable pH indicator solution.

The polymers in powder form produced local acid-neutralizing effects: sodium starch glycolate dissolved quickly with strong acid-neutralization, croscarmellose sodium dissolved quickly with weaker acid-neutralization, sodium carboxymethylcellulose did not dissolve well but exhibited some acid-neutralization, and sodium alginate exhibited little acid-neutralization (data not shown).

These results indicate that of the tested polymers, sodium starch glycolate (and to some extent, croscarmellose sodium) has a particularly strong local acid-neutralizing effect in powder form, rendering it particularly suitable for non-enterically coated formulations with an absorption enhancer such as SNAC.

Without being bound by any particular theory, it is believed that the limited effect of sodium carboxymethylcellulose and sodium alginate is associated with slow penetration of the water into the polymeric matrix, whereas the particularly effective acid neutralization of sodium starch glycolate is at least partially associated with its rapid water absorption (swelling).

As sodium starch glycolate and croscarmellose sodium exhibited the strongest local acid-neutralizing capacities, additional experiments were performed with these polymers.

To assess the maximal acid-neutralizing capacity of the polymers, experiments were repeated with each polymer compressed to a form of a tablet.

In preliminary studies, flat round 100 mg tablets were formed from sodium starch glycolate or croscarmellose sodium using a 6 mm punch and 2 ton compression force, and the tablets were placed into 40 ml HCl solutions (static dissolution) at various pH levels—1.2, 1.5 or 2.0. A temperature of 37° C. was maintained, using a water bath. The resulting pH values were determined using a suitable pH indicator solution.

Although sodium starch glycolate and croscarmellose sodium tablets exhibited different swelling patterns, they exhibited comparable neutralization strength in HCl solutions (data not shown). Sodium starch glycolate tablets resulted in stronger acid neutralization than did croscarmellose sodium tablets for each tested initial pH (wherein the efficacy of local acid neutralization was greater when the initial pH was higher).

These results indicate that although sodium starch glycolate and croscarmellose sodium may have comparable efficacy in increasing the pH of gastric fluid to at least about 4, sodium starch glycolate is more effective than croscarmellose sodium at raising the pH of gastric fluid to at least about 5, indicating that sodium starch glycolate is more effective at acid neutralization than is croscarmellose sodium.

In additional studies, 100 mg sodium starch glycolate tablets were prepared with an 8 mm round punch and 1.5-ton compression force. The tablets were dissolved without shaking in weighing boats containing 50 ml 0.01 M HCl solutions (pH 2.0, 37° C.), and local pH was determined.

Determination of pH in the proximity of the tablets, by adding an indicator to the solution, indicated that the local pH was about 7.

These results indicate that sodium starch glycolate increases local pH to well above pH 5 (the pKa of SNAC), thereby facilitating dissolution of SNAC at concentrations effective for the action of SNAC and related compounds as an absorption enhancer, as well as inhibiting peptide degradation by pepsin (which is active only at low pH).

Without being bound by any particular theory, it is believed that the local nature of the observed pH increase is associated with the viscous gel which forms upon polymer dissolution (hydration and relaxation upon wetting), which limits the transport of acid from outside to the local region with higher pH. It is further believed that due to this inhomogeneity, determination of local pH presents a more relevant parameter than pH of the medium as a whole. For example, the generally smaller increases in pH of the medium as a whole (FIGS. 3A-B) may be explained as reflecting an average of the pH of the region proximal to the tablets in which acid is neutralized effectively and the pH of distal regions in which pH is less affected.

In additional studies, 100 mg tablets were formed from various polymers (e.g., sodium starch glycolate, croscarmellose sodium, sodium carboxymethylcellulose and sodium alginate) using an 8 mm round punch and 2 ton compression force. The effects of the tablets on local pH were compared upon placement of the tablets in 0.01 M HCl solution (pH 2.0, 37° C.). pH was determined by adding an indicator to the solution.

The sodium starch glycolate tablet rapidly swelled and disintegrated, and produced a strong local acid-neutralizing effect; the croscarmellose sodium tablets also rapidly disintegrated, but produced a weaker local acid-neutralizing effect; the sodium carboxymethylcellulose tablets swelled slowly and exhibited some local acid-neutralization; and the sodium alginate tablets remained intact and exhibited little local acid-neutralization (data not shown).

In addition, a control tablet prepared with hydroxypropyl methylcellulose (HPMC), a polymer with no anionic groups, was tested in a similar manner, and exhibited no observable effect on pH.

Because of the gradual hydration of the tablets, the acid-neutralizing effect of the polymers in the swollen matrix may be inhomogeneous. The pH at the different locations of the tablet matrix was therefore assessed in additional sets of experiments, as follows.

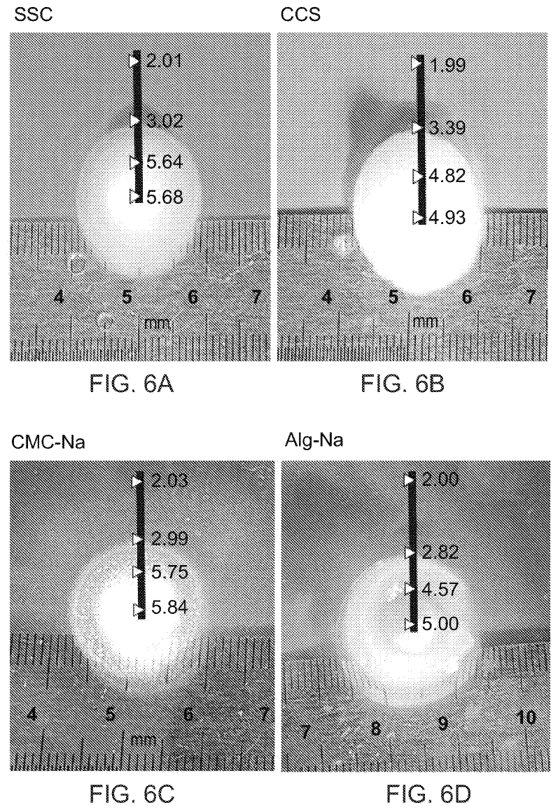

200 mg tablets were prepared from sodium alginate, Na-CMC, CCS or sodium starch glycolate (SSG) with a 10 mm round punch and 1 ton compression force. Each tablet was placed at the center of a dish containing 100 ml 0.01 M HCl solutions (pH 2.0, room temperature), and after hydration, local pH was determined at four different points of the swollen tablet matrix, as shown in FIGS. 6A (for SSG), 6B (for CCS), 6C (for Na-CMC; CMC-Na) and 6D (for sodium alginate; Alg-Na), and as follows: (i) in the HCl solution outside of the formed gel boundaries (about 2-3 cm from the swollen matrix's surface); (ii) at the surface of the swollen matrix; (iii) at an intermediate distance between the surface and the center of the swollen matrix (about 0.5-1 cm from each of the surface and the center); and (iv) at the center of the swollen matrix). Measurement of the local pH inside the tablets was enables by using a pH meter with a thin electrode (as described under the “Materials and Methods” section).

As shown in FIGS. 6A-D, the SSG tablet rapidly swelled and disintegrated, and produced a strong local acid-neutralizing effect reaching pH 5.7 in the center of the swollen matrix (FIG. 6A). The CCS tablet also rapidly swelled, and produced a slightly weaker local acid-neutralizing effect (FIG. 6B). The CMC-Na tablet swelled slowly and exhibited strong local acid-neutralization (FIG. 6C); and the Alg-Na tablet exhibited the slowest swelling rate and local acid-neutralization similar to CCS (FIG. 6D). For all the tested polymers, the pH values measured in the center of the swollen matrix was higher than other areas, further supporting a concentration-dependent acid-neutralizing capacity of the polymers.

The results of these two sets of experiments show that for all the tested alkaline polymers, an increase in the local pH inside the matrix is achieved, up to pH of about 5-6. Such pH values are in line with the desired pH for maintaining SNAC in a dissolvable salt form, as the pKa of SNAC is about 5. This pH increase thus reduces precipitation of SNAC and consequently achieves higher concentrations of dissolved SNAC which are required for its action (and other absorption enhancers) as an absorption enhancer. The local pH increase also inhibits peptide degradation by pepsin (which is active only at low pH). These results further indicate that an alkaline (basic) polymer, by exhibiting limited diffusion in acidic solution and thus affecting pH primarily in a local manner, can result in a stronger and/or more prolonged effect on pH.

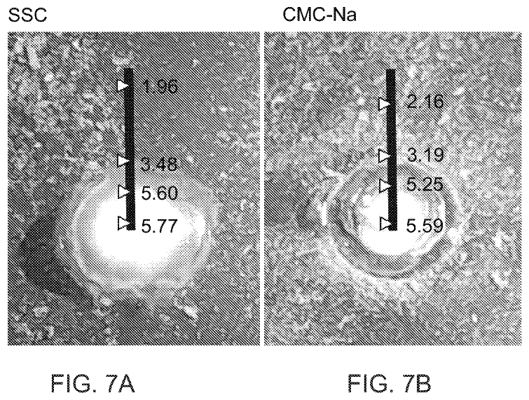

In yet another set of experiments, SSG, as an example of a fast swelling polymer that produces a high local pH, and CMC-Na, as an example of slow swelling polymer that produces a high local pH, were further tested in porcine gastric juice (withdrawn endoscopically from a sedated adult female domestic pig, Sus scrofa domesticus). 200 mg tablets were prepared from each polymer with a 10 mm round punch and 1 ton compression force. Each tablet was placed at the center of a dish containing 100 ml of the porcine gastric juice at room temperature, and after hydration (wetting), local pH was determined at four different points of the swollen tablet matrix (i)-(iv) as explained above for the data shown in FIGS. 6A-D. The results are presented in FIG. 7A for SSG and in FIG. 7B for CMC-Na and are similar to those shown in FIGS. 6A and 6C, respectively.

Taken together, the above results indicate that alkaline group-containing polymers, especially sodium starch glycolate and croscarmellose sodium, can effectively neutralize acid within a local region (e.g., within the stomach), and thereby reduce protonation and inactivation of SNAC, as well as degradation of peptides by pepsin, within such a region. This phenomenon would be of particular importance when the polymer, SNAC and a peptide or polypeptide are co-localized (e.g., within a single dosage form), allowing prolonged SNAC-induced absorption of the peptide or the polypeptide.

### Example 2

### Effect of Sodium Starch Glycolate on Peptide Absorption In Vivo Rat Model

The ability of sodium starch glycolate (SSG) to facilitate drug absorption was assessed in an in vivo rat model, using teriparatide (hPTH (1-34)) as a model peptide drug and SNAC as model absorption enhancer.

Formulations were prepared with compositions presented in Table 1. All substances were in a dry powder form and mixed geometrically using mortar and pestle. Afterwards, mini-tablets were prepared from the mixes using a 2 mm punch and die set at 0.5 ton/cm².

TABLE 1
Compositions of exemplary formulations (comprising 90 μg hPTH(1-
34), about 14.4 mg SNAC, and about 0.15 mg magnesium stearate)
administered to rats in different test groups

| | Soybean | Sodium | | |
|---|---|---|---|---|
| | trypsin | starch | Total dose | |
| | inhibitor | glycolate | weight | No. of |
| Formulation | (mg) | (mg) | (mg) | rats |
| SNAC + SBTI + 20% SSG | about 0.5 | 3.6 | 18.6 | 6 |
| SNAC + 30% SSG | 0 | 4.37 | 18.9 | 5 |
| SNAC + SBTI | about 0.5 | 0 | 15.0 | 7 |
| SNAC | 0 | 0 | 14.6 | 8 |

Male Wistar rats (250-300 grams) were divided into groups which received different formulations (as described in Table 1); and were deprived of food the night before the experiments and during the experiment, and deprived of water an hour before the experiment and during the experiment. Administration of the mini-tablets to the rats was performed using a customized intra-gastric gavage. Blood was taken from the cheek at predetermined time points. Plasma was separated and hPTH (1-34) quantified by a commercially available Elisa kit for the determination of hPTH (1-34) in plasma or cell culture media (Quidel Corp. Athens, OH, USA; Cat. No. 60-3900).

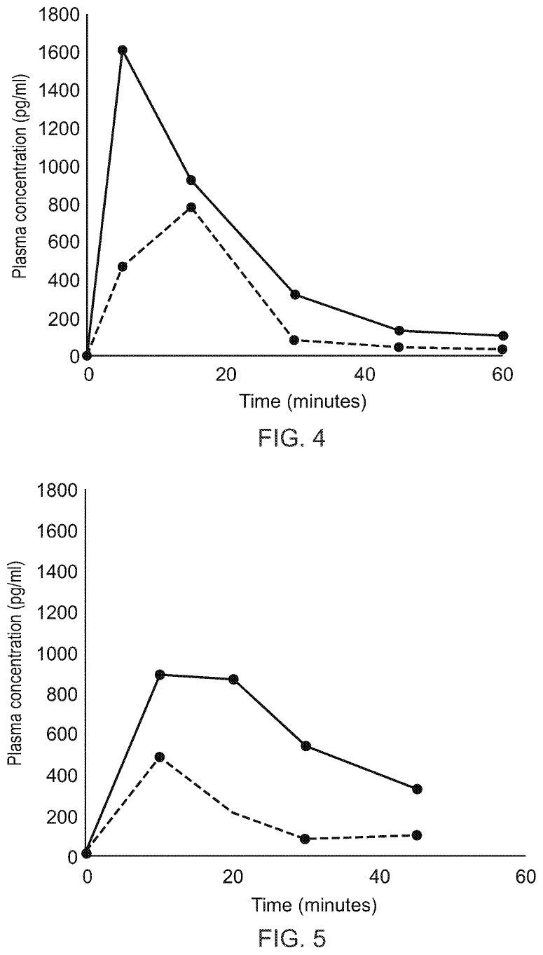

As shown in FIG. 4 and in Table 2 below, inclusion of 20% sodium starch glycolate in exemplary formulations with SBTI resulted in about a 1.5-fold greater median Cmax and AUC (area under curve), as compared to the corresponding formulation without sodium starch glycolate.

As shown in FIG. 5 and in Table 2 below, inclusion of 30% sodium starch glycolate in exemplary formulations (without SBTI) resulted in about a 4-fold greater median Cmax and AUC (area under curve), as compared to the corresponding formulation without sodium starch glycolate.

As further shown in FIGS. 4 and 5, sodium starch glycolate increased plasma levels of hPTH (1-34) during the elimination phase, indicating prolonged absorption.

TABLE 2
Pharmacokinetic parameters for hPTH(1-34) (median with range) of exemplary formulations
following oral administration of formulations to male Wistar rats

| | Cmax | | AUC(0-last) | | Tmax | | No. of |
|---|---|---|---|---|---|---|---|
| Formulation | (pg/ml) | | (minutes*pg/ml) | | (minutes) | | rats |
| SNAC + SBTI + 20% SSG | 1025 | (154-2990) | 23182 | (3731-62856) | 10 | (5-30) | 6 |
| SNAC + 30% SSG | 1633 | (330-20652) | 38015 | (7001-414106) | 20 | (10-45) | 5 |
| SNAC + SBTI | 739 | (92-1240) | 14151 | (1949-34046) | 15 | (5-15) | 7 |
| SNAC | 456 | (212-1590) | 8474 | (4416-25311) | 10 | (10-30) | 8 |

These results support those of Example 1, and confirm the ability of sodium starch glycolate to markedly increase absorption of a polypeptide co-administered with an absorption enhancer such as SNAC, and modulate the pharmacokinetic profile, both in the presence and absence of a protease inhibitor.

### Example 3

### Effect of Sodium Starch Glycolate on Peptide Absorption in an In Vivo Pig Model

The ability of sodium starch glycolate (SSG) to facilitate drug absorption was further assessed in an in vivo pig model, which allows testing of unit dosage forms with conventional dimensions for use in humans, using teriparatide (hPTH(1-34)) as a model peptide drug and SNAC as model absorption enhancer.

Formulations with or without 25% SSG were prepared with compositions presented in Table 3. All substances were in a dry powder form and mixed geometrically using mortar and pestle. Afterwards, tablets were prepared from the mixes using a manual press.

The obtained tablets were orally administered to female Sinclair minipigs (18-20 kg) on different visits. Pigs were deprived of food the night before the experiments and during the experiment and deprived of water an hour before the experiment and during the experiment. Administration was facilitated by a customized device for oral administration accompanied by about 30 ml of water. Blood was taken from the jugular vein through an indwelling cannula at predetermined time points. Plasma was separated and hPTH (1-34) was quantified using a commercially available Elisa kit for the determination of hPTH (1-34) in plasma or cell culture media (Quidel Corp. Athens, OH, USA; Cat #60-3900).

TABLE 3
Compositions of exemplary formulations administered
to pigs, comprising teriparatide (hPTH(1-34)), SNAC
and magnesium stearate, with or without 25% SSG

| | | | Mg | | Total |
|---|---|---|---|---|---|
| | hPTH(1-34) | SNAC | Stearate | SSG | weight |
| Formulation | (mg) | (mg) | (mg) | (mg) | (mg) |
| SNAC | 1 | 166 | 0.17 | — | 167 |
| 1 tablet (8 mm) | | | | | |
| SNAC + SSG | 1 | 166 | 0.17 | 55 | 222 |
| 1 tablet (8 mm) | | | | | |
| SNAC | 1 | 166 | 0.17 | — | 167 |
| 22 tablets (2 mm) | | | | | |
| SNAC + SSG | 1 | 166 | 0.17 | 55 | 222 |
| 22 tablets (2 mm) | | | | | |

As shown in Table 4, formulations containing sodium starch glycolate consistently produced higher Cmax and AUC values than the corresponding formulations without sodium starch glycolate, with plasma levels remaining higher also during the elimination phase.

These results further confirm the ability of an alkaline group-containing polymer to enhance absorption of a polypeptide in the presence of an absorption enhancer.

TABLE 4
Teriparatide (hPTH(1-34)) pharmacokinetic parameters (median ±
SEM) of exemplary formulations following oral administration to female
Sinclair minipigs (a. p < 0.05 compared to the counterpart
group by Wilcoxon signed rank test)

| | hPTH(1-34) | Cmax | AUC(0-last) | N |
|---|---|---|---|---|
| Formulation | (mg) | (pg/ml) | (min*pg/ml) | (pigs) |
| SNAC | 1 | about 95 | about 1480 | 6 |
| 1 tablet (8 mm) | | | | |
| SNAC + SSG | 1 | about 160 | about 5490 | 6 |
| 1 tablet (8 mm) | | | | |
| SNAC | 1 | about 33 | about 500 | 4 |
| 22 tablets (2 mm) | | | | |
| SNAC + SSG | 1 | about 58 | about 1300 | 4 |
| 22 tablets (2 mm) | | | | |

### Example 4

### Effect of Sodium Starch Glycolate on GLP-2 Absorption in an In Vivo Model

Teduglutide is a commercially available 33-amino acid peptide with significantly higher stability to metabolic degradation than the endogenous glucagon like peptide 2 (GLP-2). It is clinically used as a treatment for short bowel syndrome. Currently practiced methodologies employing teduglutide include subcutaneous (SC) injection, since its instability in and impermeability from the GI tract renders the drug's oral (PO) bioavailability negligible.

The effect of SNAC, as an exemplary absorption enhancer, and SSG, as an exemplary alkaline polymer, on the oral bioavailability of teduglutide was tested.

A formulation was prepared with 0.7 mg teduglutide (2.34 mg/Kg), 15 mg SNAC, 0.2 mg stearate and 5.33 mg SSG. All substances were in a dry powder form and mixed geometrically. Afterwards, mini-tablets were prepared from the mixes using a 2 mm punch and die set at 1 ton/cm².

The study was conducted in 7 male Sprague-Dawley (SD) rats weighing about 299 grams (290-320 grams). Animals were deprived of food 12-15 hours prior to administration. Water supply was removed one hour before administration and returned one hour post-administration. The mini-tablets were administered by a flexible intragastric gavage with a tip adapted for solid dosage forms administration, without the aid of water. Blood samples were thereafter collected at pre-determined time points, the plasma was separated, and the plasma level of teduglutide was quantified by an ELISA kit for the detection of endogenous GLP-2 in plasma (Abcam, Cat. No. ab222863) according to manufacturer's method Version 7, upon required modifications.

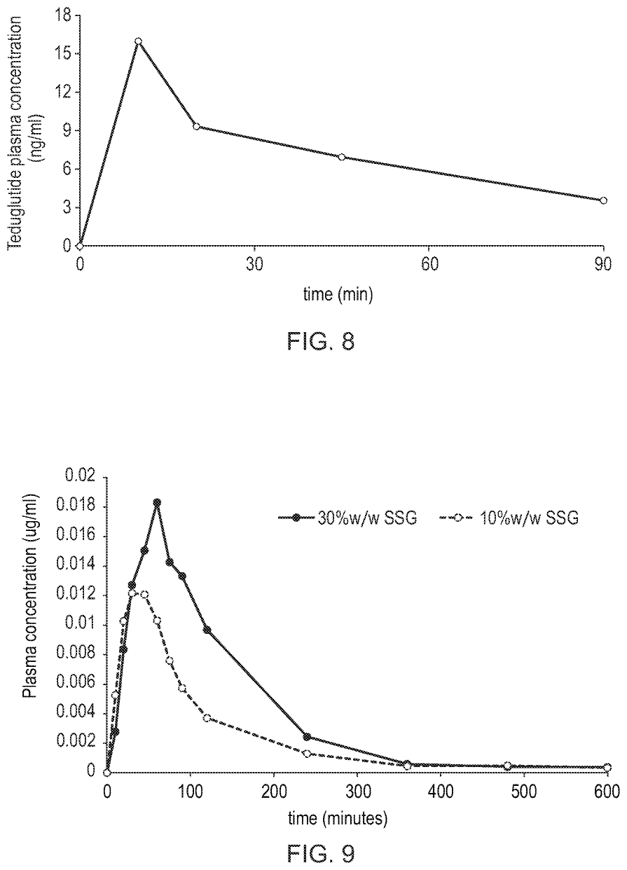

The obtained profile of teduglutide's plasma concentrations as a function of time is presented in FIG. 8, demonstrating that combination of SNAC with the buffering polymer (SSG) can be utilized to enable oral delivery of teduglutide.

### Example 5

### Effect of Sodium Starch Glycolate on Kappa Opioid Receptor Agonist Absorption in an In Vivo Model

The ability of sodium starch glycolate (SSG) to facilitate drug absorption was assessed in an in vivo dog model using an exemplary commercially available peripherally-restricted kappa opioid receptor agonist (about 1 kDa) and SNAC as model absorption enhancer.

Formulations with or without 30% SSG were prepared with compositions presented in Table 5. All substances were in a dry powder form and mixed geometrically. Afterwards, Test tablets were prepared using an 8 mm punch and die set on a manual single punch tablet compression machine. Control tablets were prepared using a 5 mm punch and die set on manual press at 0.7 ton/cm².

TABLE 5
Compositions of exemplary formulations administered
to dogs, comprising an exemplary kappa opioid receptor
agonist (about 1 kDa), denoted as “peptide”, SNAC
and excipients, with or without 30% SSG

| | Peptide | SNAC | Excipient | SSG | HEC |
|---|---|---|---|---|---|
| Formulation | (%) | (%) | (%) | (%) | (%) |
| Control | 0.8 | 92 | 2 | — | 5 |
| Test | 0.6 | 68 | 2 | 30 | — |

The study was conducted with 6 male dogs (8-11 kg, 8-9 months old). Dogs were fasted overnight with free access to water (about 14-18 hours), and were fed four hours after dosing. Three dogs were administered via an intragastric gavage a control tablet and three other dogs were administered a test tablet. 10 ml water were given after the gavage. 5% HEC (hydroxyethyl cellulose; a polymer without buffering capacity) was added to the control tablets in order to maintain a similar dissolution rate of the formulations.

Blood samples were thereafter collected at pre-determined time points, the plasma was separated, and the plasma level of the peptide was quantified by an art-acceptable method such as an ELISA kit, for the detection of the systemic exposure to the peptide.

The obtained data are presented in Table 6, and show that the inclusion of SSG in the tablet resulted in a significant increase in the AUC value, compared to the control. The obtained Tmax in both tested groups was similar (50 vs 55 minutes), indicating comparable dissolution rate of formulation in both groups.

TABLE 6

| | SSG content in | AUC | Tmax |
|---|---|---|---|
| Group | the tablet | (min*μg/ml) | (min) |
| Control | 0% w/w SSG | 1.4 | 55 |
| Test | 30% w/w SSG | 3.5 | 50 |

SSG—sodium starch glycolate. AUC values were adjusted to 0.1 mg/kg dose. N = 3 male dogs in each group. Statistically significant difference was found between the two groups (T-test, P < 0.05).

In order to further understand the effect of combining of SNAC with an alkaline polymer on the systemic exposure of the delivered peptides, an additional study was conducted in 12 dogs. 6 dogs in each of the two groups were orally administered a tablet containing the same ˜1 kDa therapeutic peptide co-formulated with SNAC and different amounts of SSG, as shown in Table 7. Tablets were prepared as described hereinabove. The study was conducted as described hereinabove and the obtained data is presented in Table 8 below and in FIG. 9.

TABLE 7

| | Peptide | SNAC | Excipient | SSG |
|---|---|---|---|---|
| Formulation | (%) | (%) | (%) | (%) |
| 1 | 0.8 | 87 | 2.6 | 10 |
| 2 | 0.6 | 68 | 2 | 30 |

TABLE 8

| | SSG content in the tablet | AUC |
|---|---|---|
| Formulation | (w/w) | (min*μg/ml) |
| 1 | 10% | 1.6 |
| 2 | 30% | 2.8 |

SSG—sodium starch glycolate. N = 6 male dogs in each group. Statistically significant difference was found between the two groups (T-test, P < 0.05).

As can be seen in Table 8 and FIG. 9, the systemic exposure (AUC) was higher in tablets comprising a higher SSG content (formulation 2).

### Example 6

### Effect of a Combination of SSG and Small Proton-Accepting Agents in Gastric Conditions

The present inventors have studied the effect of combining the buffering polymers as described herein with non-polymeric (small), acid-neutralizing agent (small proton-accepting agents; sPAA) on the protection of SNAC and the therapeutically active agent in gastric fluid. Under gastric conditions (in the stomach), where the tablet disintegrates and its content is released and dissolved, the released SNAC diffuses away from the buffered center of the formulation matrix to the acidic periphery of the matrix, which, as explained herein, reduces the solubility and hence the activity of SNAC. The present inventors have conceived overcoming this possible pathway by the addition of small molecules that act as proton acceptors, and which can diffuse away from the core of the tablet at approximately the same kinetics as SNAC, and thus prevent SNAC precipitation at the periphery of the tablet (outside the core where the polymers exhibit the buffering effect). The present inventors have conceived that including such agents in the composition may allow using lower amounts of SNAC. Thus, according to some of any of the embodiments described herein, an effective amount of an absorption enhancer such as SNAC in the composition, is lower by at least 10%, or by at least 20%, or by at least 30%, compared to an effective amount in the same composition that does not include the small molecule proton accepting agent.

Studies were conducted using several types of proton accepting small molecules (non-polymeric acid-neutralizing agents): antacids, alkaline amino acids (having a side change featuring pKa higher than 2, preferably higher than 3, more preferably higher than 4 or higher than 5), and fatty acid salts (featuring pKa higher than 2, preferably higher than 3, more preferably higher than 4).

Each type of the tested proton accepting small molecule agents, or a combination of two types of proton accepting small molecule agents, was tested at various concentrations and various mol ratios of the small proton accepting agent (sPAA) to SNAC as indicated, and the soluble fraction of SNAC in a medium mimicking fasted stomach at different time points was determined, as described under the Methods section hereinabove.

### Determination of Soluble Fraction of SNAC in Formulations Containing SSG and Antacids:

Table 9 below presents the tested formulations.

TABLE 9

| | | SNAC | sPAA | SNAC:sPAA |
|---|---|---|---|---|
| Formulation | SSG (mg) | (mg) | (mg) | mol ratio |
| SSG-SNAC-Na₂CO₃ | 20 | 80 | 28.1 | 1:1 |
| SSG-SNAC-Na₂CO₃ | 20 | 80 | 14.1 | 1:0.5 |
| SSG-SNAC-Na₂CO₃ | 20 | 80 | 7.0 | 1:0.25 |
| SSG-SNAC-K₂CO₃ | 20 | 80 | 36.6 | 1:1 |
| SSG-SNAC-K₂CO₃ | 20 | 80 | 18.3 | 1:0.5 |
| SSG-SNAC | 20 | 80 | — | — |

FIG. 10A presents the data obtained for tablets containing sodium carbonate and FIG. 10B presents the data obtained for tablets containing potassium carbonate.

As shown in FIG. 10A, the incorporation of Na₂CO₃ (sodium carbonate) into SNAC-SSG formulations significantly enhanced the protective effect, in a dose-dependent manner. Formulations with a SNAC: Na₂CO₃ mol ratio of 1:1 achieved approximately 100 percent recovery of SNAC by protecting it from precipitation. Formulations containing SNAC-SGG: Na₂CO₃, mol ratios of 0.5 and 0.25 showed reduced efficacy but still exhibited approximately ×4 and ×2.5 recovery, respectively, compared to a formulation with SSG only.

As shown in FIG. 10B, the incorporation of K₂CO₃ into SNAC-SSG formulations significantly improved protective efficacy. A formulation containing a SNAC-SSG: K₂CO₃ mol ratio of 1:1 resulted in approximately 100 percent SNAC recovery, while a ratio of 0.5 achieved lower efficacy, but still quadrupled the recovery compared to a formulation with only SSG.

### Determination of Soluble Fraction of SNAC in Formulations Containing SSG and Fatty Acid Salts:

Table 10 below presents the tested formulations.

TABLE 10

| | | SNAC | sPAA | SNAC:sPAA |
|---|---|---|---|---|
| Formulation | SSG (mg) | (mg) | (mg) | mol ratio |
| SSG-SNAC-Sodium | 20 | 80 | 51.6 | 1:1 |
| Decanoate (C₁₀) | | | | |
| SSG-SNAC-Sodium | 20 | 80 | 25.8 | 1:0.5 |
| Decanoate (C₁₀) | | | | |
| SSG-SNAC-Sodium | 20 | 80 | 12.9 | 1:0.25 |
| Decanoate (C₁₀) | | | | |
| SSG-SNAC-Sodium | 20 | 80 | 44.1 | 1:1 |
| Octanoate (C₈) | | | | |
| SSG-SNAC | 20 | 80 | — | — |

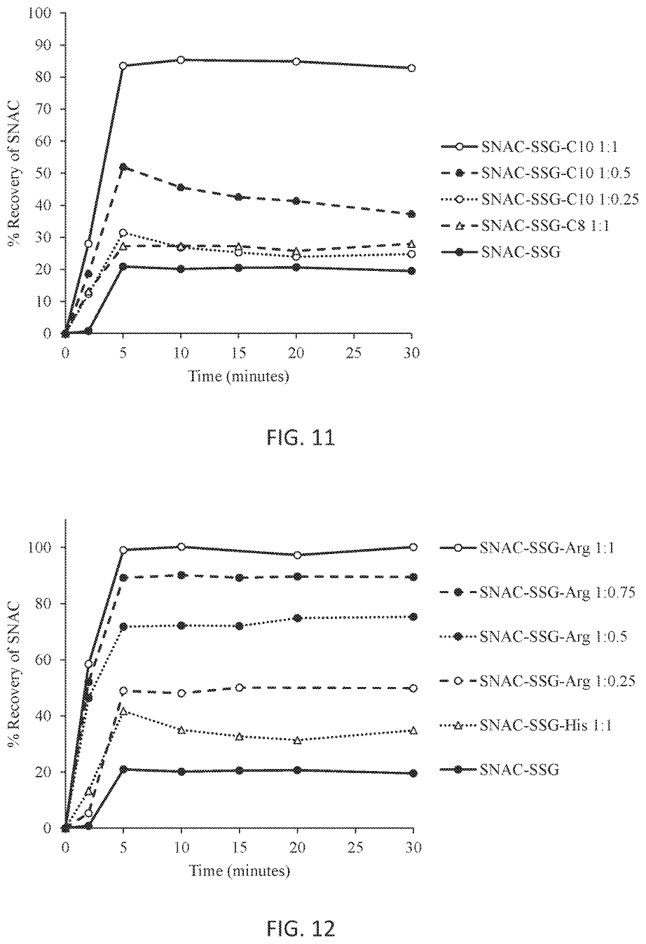

The obtained data is presented in FIG. 11 and show that the incorporation of fatty acid salts to the SNAC-SSG formulation significantly enhanced the protective effect. The SNAC-SSG formulation containing sodium decanoate (C₁₀) fatty acid salt at a mol ratio to SNAC of 1:1 achieved approximately 80 percent recovery of SNAC, while formulations with mol ratios of 1:0.5 and 1:0.25 exhibited a recovery of approximately 40 and 25 percent, respectively, over 30 minutes. A SNAC-SSG formulation containing sodium octanoate (C₈) at a mol ratio to SNAC of 1:1 showed less efficacy but still achieved approximately a 1.5-fold increase in recovery compared to a formulation with only SSG.

### Determination of Soluble Fraction of SNAC in Formulations Containing SSG and Amino Acids:

Table 11 below presents the tested formulations.

| | SSG | SNAC | sPAA | SNAC:sPAA |
|---|---|---|---|---|
| Formulation | (mg) | (mg) | (mg) | mol ratio |
| SSG-SNAC-Arginine (Arg) | 20 | 80 | 46.3 | 1:1 |
| SSG-SNAC-Arg | 20 | 80 | 34.7 | 1:0.75 |
| SSG-SNAC-Arg | 20 | 80 | 23.1 | 1:0.5 |
| SSG-SNAC-Arg | 20 | 80 | 11.6 | 1:0.25 |
| SSG-SNAC-Histidine (His) | 20 | 80 | 41.2 | 1:1 |
| SSG-SNAC | 20 | 80 | — | — |

The obtained data is presented in FIG. 12 and show that the incorporation of alkaline (basic) amino acids as described herein to the SNAC-SSG formulations significantly increased the protective effect, in a dose-dependent manner. Formulations containing L-arginine at molar ratios of 1:1, 1:0.75, 1:0.5, and 1:0.25 achieved approximately 100, 90, 75, and 50 percent recovery, respectively. A SNAC-SSG formulation containing histidine at a molar ratio of 1:1 achieved a lower recovery of approximately 40 percent, indicating that amino acids that feature higher pKa values of the side chain exhibit a more potent effect.

Determination of Soluble Fraction of SNAC in Formulations Containing SSG and a Combination of sPAAs:

The effect of combinations of two proton-accepting agents, each of a different type, was tested, as shown on Table 12.

TABLE 12

| | | | 1^{st} | 2^{nd} | |
|---|---|---|---|---|---|
| | SSG | SNAC | sPAA | sPAA | SNAC:sPAA |
| Formulation | (mg) | (mg) | (mg) | (mg) | mol ratio |
| SSG-SNAC-C₈- | 20 | 80 | 44.1 | 41.2 | 1:1:1 |
| Histidine | | | | | |
| SSG-SNAC-His | 20 | 80 | 41.2 | — | 1:1 |
| SSG-SNAC C₈ | 20 | 80 | 44.1 | — | 1:1 |
| SSG-SNAC-Na₂CO₃- | 20 | 80 | 7.0 | 11.6 | 1:0.25:0.25 |
| Arginine | | | | | |
| SSG-SNAC-Na₂CO₃ | 20 | 80 | 7.0 | — | 1:0.25 |
| SSG-SNAC-Arginine | 20 | 80 | 11.6 | — | 1:0.25 |
| SSG-SNAC | 20 | 80 | — | — | — |

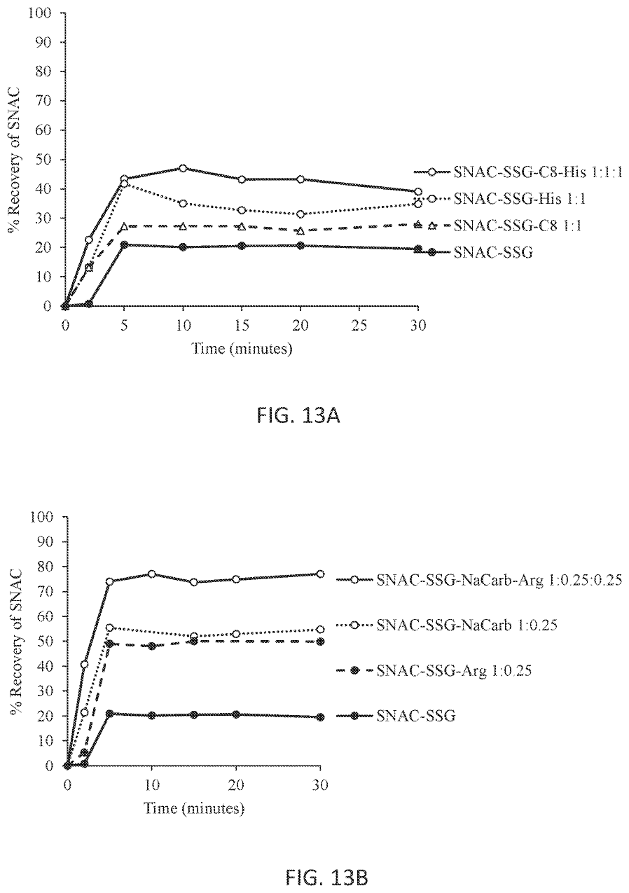

As shown in FIG. 13A, SNAC-SSG formulations incorporating histidine individually or in combination with C8, at mol ratios of 1:1 and 1:1:1, respectively, achieved approximately 40 percent recovery over 30 minutes, indicating that the combination of histidine with C₈ does not improve the SNAC recovery.

As shown in FIG. 13B, SNAC-SSG formulations containing Na₂CO₃ and Arginine in combination, at mol ratios of 1:0.25:0.25, increased the protective effect compared to formulations containing Na₂CO₃ and Arginine individually at similar mol ratios. Specifically, the SNAC-SSG formulation containing a combination of Na₂CO₃ and Arginine, at mol ratios of 1:0.25:0.25, achieved an approximate 80 percent recovery of SNAC, whereby the formulations containing Na₂CO₃ and Arginine separately, in similar mol ratios, achieved approximately 55 and 50 percent recovery, respectively.

Overall, these data show that a combination of SNAC with a combination of polymeric and non-polymeric acid-neutralizing substances represents a novel and improved approach for oral delivery of therapeutically active agents such as peptides and proteins, which is otherwise limited, and moreover significantly improves the oral bioavailability of the therapeutically active agents.

### In Vivo Studies:

The ability of small molecules proton accepting agent(s) to further increase the bioavailability of a therapeutically active agent co-formulated with an alkaline polymer and an absorption enhancer as described herein, was assessed in an in vivo pig model.

Test animals were divided into groups, and each animal was administered intragastrically by endoscopic device orally with either a control formulation, comprised of SNAC and an alkaline polymer (e.g., SSG) and a selected therapeutically active agent, or a test formulation, comprised of SNAC, the alkaline polymer (e.g., SSG), the selected therapeutically active agent and a selected small molecule proton-accepting agent as described herein, at a 1:1 mol ratio to the SNAC.

The effect of combination of SNAC, sodium starch glycolate (SSG) as an exemplary alkaline polymer and a small molecule proton-accepting agent (Sodium carbonate, arginine and/or Sodium Decanoate (C10)) on absorption of a therapeutically active agent was assessed in an in vivo pig model, which allows testing of orally administered unit dosage forms with conventional dimensions that are applicable also in humans. The following proteinaceous therapeutically active agents were tested: lixisenatide, octreotide, desmopressin, salmon calcitonin, PTH (1-34), leuprolide.

All formulation constituents were in a dry powder form and mixed geometrically using mortar and pestle. Thereafter, round flat tablets were prepared from the mixes using a punch and dye and dental manual press.

The obtained tablets were administered at monitored conditions with the aid of endoscope into the stomachs of 2 male and 2 female mini-pigs (about 45 kg) for each tested group on different visits. Pigs were deprived of food the night before the experiments and during the experiment and deprived of water an hour before the experiment and during the experiment.

Blood samples were taken from the jugular vein through an indwelling cannula at predetermined time points during the first hour post-dose (which reflects the absorption phase). Plasma was separated and the concentration of the tested model molecules in plasma was quantified using a suitable technique.

hPTH (1-34) was quantified using a commercially available Elisa kit for the determination of hPTH (1-34) in plasma or cell culture media (Quidel Corp. Athens, OH, USA; Cat #60-3900).

Octreotide was quantified using a commercially available Elisa kit for the determination of octreotide in plasma or cell culture media (BMA Biomedicals, Augst, Switzerland. Cat #S-1341).

Desmopressin was quantified using a commercially available Elisa kit for the determination of desmopressin in plasma or cell culture media, such as, for example, marketed by BMA Biomedicals, Augst, Switzerland (e.g., Cat #S-1364).

Leuprolide was quantified using a commercially available Elisa kit for the determination of leuprolide in plasma or cell culture media, such as, for example, marketed by BMA Biomedicals, Augst, Switzerland (e.g., Cat #S-1144.

Salmon calcitonin was quantified using a commercially available Elisa kit for the determination of salmon calcitonin in plasma or cell culture media, such as, for example, marketed by BMA Biomedicals, Augst, Switzerland (e.g., Cat #S-1155).

Lixisenatide was quantified by a commercially available Elisa kit for the determination of lixisenatide in plasma or cell culture media and/or by a validated LC-MS/MS method.

Formulation of the therapeutically active agents desmopressin (T1/2 (IV half-life)=3-5 hours); Salmon calcitonin (IV T1/2=60-90 minutes); Leuprolide (IV T1/2=about 3 hours); lixisenatide (IV T1/2=about 3 hours); hPTH (1-34) (T1/2=about 5 minutes); and octreotide (IV T1/2=about 1.5 hour), with SNAC and SSG typically included about 0.3-0.5% by weight of the active agent, 75-80% by weight SNAC, about 22% by weight SSG and magnesium stearate as a lubricant (about 1% by weight), and the total weight of the tablet was 100-110 mg.

Formulations of these active agents with sodium carbonate as an exemplary PAA typically included about 0.3-0.5% by weight of the active agent, 55-60% by weight SNAC, about 22% by weight SSG, about 20% sodium carbonate, and magnesium stearate as a lubricant (about 1% by weight), and the total weight of the tablet was 170-180 mg.

Formulations of these active agents with C10 as an exemplary PAA typically included about 0.3-0.5% by weight of the active agent, 45-50% by weight SNAC, about 22% by weight SSG, about 30% sodium decanoate (C10), and magnesium stearate as a lubricant (about 1% by weight), and the total weight of the tablet was 170-180 mg.

Formulations of these active agents with arginine as an exemplary PAA typically included about 0.3-0.5% by weight of the active agent, 45-50% by weight SNAC, about 22% by weight SSG, about 25-30% arginine, and magnesium stearate as a lubricant (about 1% by weight), and the total weight of the tablet was 170-180 mg.

Formulation of the therapeutically active agents desmopressin (T1/2 (IV half-life)=3-5 hours); Salmon calcitonin (IV T1/2=60-90 minutes); Leuprolide (IV T1/2=about 3 hours); lixisenatide (IV T1/2=about 3 hours); hPTH (1-34) (T1/2=about 5 minutes); and octreotide (IV T1/2=about 1.5 hour), with SNAC and SSG typically included about 0.3-0.5% by weight of the active agent, 75-80% by weight SNAC, about 22% by weight SSG and magnesium stearate as a lubricant (about 1% by weight), and the total weight of the tablet was 100-110 mg.

Formulations containing the same therapeutically active agents were also formulated with with 11% SSG and typically included about 0.3-0.5% by weight of the active agent, 85-90% by weight SNAC, and magnesium stearate as a lubricant (about 1% by weight), and the total weight of the tablet was 90-95 mg.

Formulations of these active agents with lower SSG amount and with sodium carbonate as an exemplary PAA typically included about 0.3-0.5% by weight of the active agent, 60-65% by weight SNAC, about 11% by weight SSG, 20-25% sodium carbonate, and magnesium stearate as a lubricant (about 1% by weight), and the total weight of the tablet was 120-130 mg.

Formulations of these active agents with C10 as an exemplary PAA typically included about 0.3-0.5% by weight of the active agent, 50-55% by weight SNAC, about 11% by weight SSG, about 30-40% sodium decanoate (C10), and magnesium stearate as a lubricant (about 1% by weight), and the total weight of the tablet was 150-160 mg.

Formulations of these active agents with arginine as an exemplary PAA typically included about 0.3-0.5% by weight of the active agent, 50-60% by weight SNAC, about 11% by weight SSG, about 30-35% arginine, and magnesium stearate as a lubricant (about 1% by weight), and the total weight of the tablet was 140-150 mg.

In preliminary data obtained in these studies, it was uncovered that the mean Cmax (ng/ml) of formulations containing 0.3-0.5% by weight of octreotide, 85-90% by weight SNAC, about 11% by weight SSG, and magnesium stearate as a lubricant (about 1% by weight) was 2.09, whereby the Cmax of a SSG 11% by weight formulation which further included sodium decanoate (C10) as described herein was 2.94, indicating the improved effect imparted by the PAA.

In further preliminary data obtained in these studies, it was uncovered that the mean AUClast (min·ng/ml) of formulations containing 0.3-0.5% by weight of pPTH1-34 (teriparatide), 85-90% by weight SNAC, about 11% by weight SSG, and magnesium stearate as a lubricant (about 1% by weight) was 5.87, whereby the AUClast of a SSG 11% by weight formulation which further included sodium decanoate (C10) as described herein was 9.87, further substantiating the improved effect imparted by the PAA.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. An oral pharmaceutical composition comprising a therapeutically active agent, an absorption enhancer comprising a substituted fatty acid or a salt thereof, a polymer comprising a plurality of alkaline groups, and at least one proton accepting non-polymeric agent, wherein a concentration of the polymer in the composition is at least 10 weight percent of the total weight of the composition and wherein the ratio (by weight) of the proton accepting non-polymeric agent to the absorption enhancer ranges from 0.2:1 to 1:1.

2. The composition of claim 1, wherein the alkaline groups in the polymer are carboxylate groups and/or amine groups.

3-4. (canceled)

5. The composition of claim 2, wherein at least a portion of the carboxylate groups in the polymer are in a form of a sodium salt.

6. The composition of claim 1, wherein the absorption enhancer is NAC (8-N-(2-hydroxybenzoyl)aminocaprylic acid), NAD (10-N-(2-hydroxybenzoyl)aminodecanoic acid), 5-CNAC (8-N-(5-chlorosalicyloyl)aminocaprylic acid), 4-MOAC (8-N-(2-hydroxy-4-methoxybenzoyl)aminocaprylic acid), or 4-CNAB (4-N-(2-hydroxy-4-chlorobenzoyl)aminobutanoic acid) or salts thereof of any of the above.

7. The composition of claim 6, wherein the absorption enhancer comprises NAC or a salt thereof.

8. The composition of claim 7, wherein a concentration of the absorption enhancer is at least 50 weight percent of the total weight of the composition.

9-12. (canceled)

13. The composition of claim 1, wherein the polymer comprises a polysaccharide.

14. The composition of claim 13, wherein the polysaccharide is a starch derivative and a cellulose derivative.

15-16. (canceled)

17. The composition of claim 14, wherein the polymer is sodium starch glycolate or croscarmellose sodium.

18-20. (canceled)

21. The composition of claim 1, wherein the at least one proton-accepting non-polymeric agent features a pKa of at least 5.

22. (canceled)

23. The composition of claim 21, wherein the at least one proton accepting non-polymeric agent is an antacid, an amino acid, a salt of a fatty acid, or a combination thereof.

24. The composition of claim 23, wherein the antacid is calcium carbonate, calcium gluconate, calcium citrate, sodium carbonate, sodium bicarbonate, sodium hydroxide, potassium carbonate, potassium bicarbonate, potassium gluconate, potassium citrate, potassium hydroxide, magnesium carbonate, magnesium gluconate, magnesium citrate, magnesium hydroxide, magnesium oxide, aluminum carbonate, aluminum gluconate, aluminum citrate, aluminum hydroxide, or any combinations thereof.

25. The composition of claim 24, wherein the at least one proton-accepting non-polymeric agent comprises sodium carbonate or potassium carbonate.

26-27. (canceled)

28. The composition of claim 25, wherein the at least one proton accepting non-polymeric agent is histidine, arginine, lysine, an analog thereof, or any combinations thereof.

29-30. (canceled)

31. The composition of claim 23, wherein the salt of a fatty acid is a salt of a medium-chain fatty acid, having from 8 to 10 carbon atoms in length.

32. The composition of claim 31, wherein the salt of a fatty acid is sodium octanoate, sodium decanoate, or a combination thereof.

33-34. (canceled)

35. The composition of claim 1, wherein a total concentration of the at least one proton-accepting non-polymeric agent in the composition is at least 20 weight percent of the total weight of the composition.

36. The composition of claim 1, wherein a Cmax of the composition upon oral administration is at least 50% greater than a Cmax of a corresponding composition without the polymer and the proton accepting non-polymeric agent and/or
wherein a bioavailability of the composition upon oral administration is at least 50% greater than a bioavailability of a corresponding composition without the polymer and the proton accepting non-polymeric agent.

37. (canceled)

38. The composition of claim 1, wherein the therapeutically active agent has a molecular weight in a range of 0.5 kDa to 100 kDa.

39. (canceled)

40. The composition of claim 38, wherein the therapeutically active agent is a polypeptide or a nucleic acid.

41. The composition of claim 40, wherein the polypeptide is a parathyroid hormone (PTH), a long-acting analog of PTH, a fragment of PTH, a long-acting analog of a fragment of PTH, GLP-1 (glucagon-like peptide-1) or a long-acting analog thereof, a GLP-1 receptor agonist or a long-acting analog thereof, a GLP-2 (glucagon-like peptide-2) or a long-acting analog thereof, a GLP-2 receptor agonist or a long-acting analog thereof, GLP-1 receptor/glucagon receptor dual agonist or a long-acting analog thereof, a GIP receptor/GLP-1 receptor dual agonist or a long-acting analog thereof, glucagon receptor/GIP receptor/GLP-1 receptor triple agonist or a long-acting analog thereof, GLP-2 receptor/glucagon receptor dual agonist or a long-acting analog thereof, GLP-2 receptor/GLP-1 receptor dual agonist or a long-acting analog thereof, GLP-2 receptor/GIP receptor dual agonist or a long-acting analog thereof, or oxyntomodulin or a long-acting analog thereof.

42. The composition of claim 41, wherein the polypeptide is characterized by isoelectric point (pl) of at least 8.5.

43-44. (canceled)

45. A unit dosage form comprising the composition of claim 1.

46. The unit dosage form of claim 45, wherein an amount of the alkaline groups in the unit dosage form is at least 0.03 millimoles.

47. The unit dosage form of claim 46, wherein the unit dosage form comprises at least 50 mg of the absorption enhancer.

48. The unit dosage form of claim 47, wherein the unit dosage form comprises at least 10 mg of the proton-accepting non-polymeric agent.

49. The unit dosage form of claim 48, wherein the unit dosage form comprises one or more tablet(s).

50. A method of treating a condition treatable by the composition of claim 1, the method comprising orally administering the composition to a subject in need thereof.
